(19) United States
(12) Patent Application Publication
(10) Pub. No.: US 2007/0192900 A1
(43) Pub. Date: Aug. 16, 2007

(54) Production of beta-glucosidase, hemicellulase and ligninase in E1 and FLC-cellulase-transgenic plants

(75) Inventor: Masomeh B. Sticklen, East Lansing, MI (US)

Correspondence Address: Ian C. McLeod;McLeod & Moyne, P.C., 2190 Commons Parkway, Okemos, MI, 48864, US

(73) Assignee: Board of Trustees of Michigan State University, East Lansing, MI (US)

(21) Appl. No.: 11/489,234

(22) Filed: Jul. 19, 2006

Related U.S. Application Data
(63) Continuation-in-part of application No. 11/451,162, filed on Jun. 12, 2006, now abandoned.
Application No. 11/451,162 is a continuation-in-part of application No. 11/354,310, filed on Feb. 14, 2006.

(51) Int. Cl. A01H 5/00; C12P 19/02; C12N 15/82

(52) U.S. Cl. 800/280; 800/284; 800/320; 800/320.1; 800/320.2; 435/105

(57) ABSTRACT

The present invention provides transgenic plants expressing one or more cell wall degrading enzymes that can degrade lignocellulose to fermentable sugars. These fermentable sugars can further be fermented to ethanol or other products. The enzymes are directed to the plastids or the apoplasts or the transgenic plant for storage. When the transgenic plants are harvested, the plants are ground to release the enzymes which then are used to degrade the lignocellulose of plant material to produce the fermentable sugars. The transgenic plants express the flowering locus c gene so that flowering is delayed and the plant biomass is increased.

### CROSS-REFERENCE TO RELATED APPLICATIONS

This application is a continuation-in-part of U.S. patent application Ser. No. 11/451,162 filed Jun. 12, 2006, which is a continuation-in-part of U.S. patent application Ser. No. 11/354,310 filed Feb. 14, 2006, and claims benefit of U.S. patent application Ser. No. 09/981,900, filed Oct. 18, 2001, and Provisional Application No. 60/242,408 filed Oct. 20, 2000.

### STATEMENT REGARDING FEDERALLY SPONSORED RESEARCH OR DEVELOPMENT

Not Applicable.

### REFERENCE TO A “NUCLEOTIDE/AMINO ACID SEQUENCE LISTING APPENDIX SUBMITTED ON A COMPACT DISC”

The application contains nucleotide and amino acid sequences which are identified with SEQ ID NOs. A compact disc is provided which contains the Sequence Listings for the sequences. The Sequence Listing on the compact disc and is identical to the paper copy of the Sequence Listing provided with the application.

### BACKGROUND OF THE INVENTION

(1) Field of the Invention

The present invention relates to transgenic plants. The transgenic plants are capable of expressing one or more cell wall degrading enzymes and a flowering locus c gene coding region. The cell wall degrading enzyme are directed to a plastid, vacuole, vesicle, cytosol or apoplast of the transgenic plant. The flowering locus c gene delays flowering while increasing biomass and enabling isolation of increased amounts of the hydrolyzing enzyme from the transgenic plant as compared to a non-transgenic plant from which the transgenic plant is derived.

(2) Description of Related Art

If human economies are to become more truly sustainable, we will need to learn how to use the solar energy and carbon fixed in plant biomass to meet a much larger fraction of our energy and raw material needs. The potential of plant biomass to help these needs is certainly real: approximately 180 billion tons of new plant matter is produced annually across the globe, or about 30 tons per person on the planet per year. (Khan, A., Jameel, A. M., Jameel, M. (1984). Energy from Biomass: Resources and expectations. In: Renewable Energy Sources: International Progress, Part B, ed. T. N. Veziroglu, pp. 87-98). The energy value of this plant matter is roughly equivalent to 10 times the total human use of all types of energy. However, because of the difficulty in extracting the energy from plant biomass, most of the energy potential of the biomass goes unused.

Much research and engineering remains to be done to actually realize the potential of plant matter to meet a greater portion of our fuel and raw material needs. Specifically, cellulose and hemicellulose are polymers of five and six carbon sugars that represent approximately 70-80% of most plant matter. These could be converted into fermentable sugars, and the rest be used for other purposes. These sugars could form the raw material and energy basis for a renewable chemical and fuel industry if the sugars could be made available at significantly lower cost.

It is encouraging that in recent years, much progress has been made toward realizing this goal of reduced cost of production of sugars from biomass. In particular, hydrolysis methods have been improved (Vlasenko E. and Chemy J. (2005). Improving cellulose hydrolysis with the new cellulase compositions. Proceedings of the 27th Symposium on Biotechnology for Fuels and Chemicals. Denver, May 1-4, 2005. Page 6: 1B-03) and recombinant microorganisms have been developed that can ferment the mixed five and six carbon sugars from plant biomass to ethanol in high yield (Moniruzzaman M., Dien B. S., Ferrer B., Hespel R. B. Dale B. E., Ingram L. O., and Bothast R. J. (1996). Ethanol production from AFEX pretreated corn fiber by recombinant bacteria. Biotechnology Letters. 18 (8): 985-990) and their efficiency has been increased (Chemy J. R. (2005). Progress on enzymes for biomass utilization and prospects for the future. Proceedings of the 27th Symposium on Biotechnology for Fuels and Chemicals. Denver, May 1-4, 2005. Page 35: CA-11). Also, different promising pretreatments have been developed and compared that make the cellulose and hemicellulose much more reactive and accessible to hydrolytic enzymes.

Scientists have made important strides in reducing the costs of production of hydrolysis enzymes through molecular enzymology and other molecular techniques. However the costs of these enzymes produced from microbes in conventional deep tank fermentation systems is still far too high to meet the economics of the commercial production of biofuels from plant biomass. An alternative technology to be tested is production of high levels of biologically active hydrolysis enzymes directly in biomass plants.

Much of the cellulose in plant biomass is in the form of lignocellulose. Lignin is a complex macromolecule consisting of aromatic units with several types of inter-unit linkages. In the plant, the lignin physically protects the cellulose polysaccharides in complexes called lignocellulose. To degrade the cellulose in the lignocellulose complexes, the lignin must first be degraded. While lignin can be removed in chemi-mechanical processes that free the cellulose for subsequent conversion to fermentable sugars, the chemi-mechanical processes are expensive and inefficient. Ligninase and cellulase enzymes, which are produced by various microorganisms, have been used to convert the lignins and cellulose, respectively, in plant biomass to fermentable sugars. However, the cost for these enzymes is expensive. As long as the cost to degrade plant biomass remains expensive, the energy locked up in the plant biomass will largely remain unused.

An attractive means for reducing the cost of degrading plant biomass is to make transgenic plants that contain cellulases. For example, WO 98/11235 to Lebel et al. discloses transgenic plants that express cellulases in the chloroplasts of the transgenic plants or transgenic plants wherein the cellulases are targeted to the chloroplasts. Preferably, the cellulases are operably linked to a chemically-inducible promoter to restrict expression of the cellulase to an appropriate time. However, because a substantial portion of the cellulose in plants is in the form of lignocellulose, extracts from the transgenic plants are inefficient at degrading the cellulose in the lignocellulose.

U.S. Pat. Nos. 5,981,835 and 6,818,803 to Austin-Phillips et al. discloses transgenic tobacco and alfalfa which express the cellulases E2, or E3 from Thermomononospora fusca. The genes encoding the E2 or E3, which were modified to remove their leader sequence, were placed under the control of a constitutive promoter and stably integrated into the plant genome. Because the leader sequence had been removed, the E2 or E3 product preferentially accumulated in the cytoplasm of the transgenic plants. However, when produced at high level in cytoplasm, the heterologous enzyme will interact with normal cytoplasmic metabolic activities and the growth of the transgenic plants can be impaired.

U.S. Pat. No. 5,536,655 to Thomas et al. discloses a gene encoding Acidothermus cellulolyticus E1 endoglucanase and corresponding protein sequences. U.S. Pat. No. 6,013,860 to Himmel et al. discloses transgenic plants which express the cellulase E1 from Acidothermus cellulolyticus. The gene encoding E1, which was modified to remove the leader region, was placed under the control of a plastid specific promoter and preferably integrated into the plastid genome. Because the leader sequence had been removed, the E1 product accumulated in the plastid. U.S. Patent Application Publication Nos. 2003/0109011 and 2006/0026715 to Hood et al. teach expression of recombinant polysaccharide degrading enzymes in plants.

The accumulation of hydrolytic enzymes in the cytoplasm of a plant is undesirable since there is the risk that the cellulase will interfere with cytoplasmic biochemical activities causing harm to the plant growth and development. For example, research has shown that plants such as the avocado, bean, pepper, peach, poplar, and orange also contain cellulase genes, which are activated by ethylene during ripening and leaf and fruit abscission. Therefore, transgenic plants which contain large quantities of cellulase in the cytoplasm are particularly prone to damage. Furthermore, the cellulases accumulate in all tissues of the plant which can be undesirable. Restriction of cellulase expression to plastids or apoplast is desirable because it reduces the risk of plant damage due the cellulases interfering with the cytoplasmic chemical reactions. However, for most crop plants, it has been difficult to develop a satisfactory method for introducing heterologous genes into the genome of plastids.

For production of ligninases to use in degrading lignins, the ligninases of choice are from the white-rot fungus Phanerochaete chrysosporium. One of the major lignin-degrading, extracellular enzymes produced by P. chrysosporium is lignin peroxidase (LIP). Potential applications of LIP include not only lignin degradation but also biopulping of wood and biodegradation of toxic environmental pollutants. To produce large quantities of LIP, the fungus can be grown in large reactors and the enzyme isolated from the extracellular fluids. However, the yields have been low and the process has not been cost-effective. Production of recombinant LIP in E. coli, in the fungus Trichoderma reesei, and baculovirus have been largely unsuccessful. Heterologous expression of lignin-degrading manganese peroxidase in alfalfa plants has been reported; however, the transgenic plants had reduced growth and expression of the enzyme was poor (Austin et al., Euphytica 85: 381-393 (1995)).

Finally, U.S. Pat. No. 6,693,228 to Amasino et al., hereby incorporated herein by reference in its entirety, discloses Flowering Locus C (flc) genes, and teaches the use of flc to delay flowering in transgenic plants as compared to non-transgenic plants. Amasino et al. disclose Flowering Locus C (flc) genes from Arabidopsis thaliana and B. rapa. Amasino et al. also teach overexpression of the A. thaliana gene under control of the constitutive 35S promoter in A. thaliana is sufficient to delay flowering in transformed plants. U.S. Patent Application Publication No. 2004/0126843 A1 to Demmer et al. suggest that the ability to control flowering in C3 monocotyledonous plants, such as forage grasses and cereals has wide ranging applications. Demmer et al. propose that controlling flowering offers the ability to control the spread of genetically modified organisms. Demmer et al. mentions genes such as flc as one of a number of genes important in regulating flowering time. U.S. Patent Application Publication No. 2004/0045049 A1 to Zhang generally teaches flc and transgenic plants having modified traits.

Thus, a need remains for improved transgenic plants expressing enzymes that degrade cellulose, hemicellulose, and/or lignin in lignocellulose to fermentable sugars. The ability to control the spread of the transgenic plants is important.

### SUMMARY OF THE INVENTION

The present invention provides transgenic plants expressing cell wall degrading enzymes that degrade cellulose, hemicellulose and/or lignin in lignocellulose to fermentable sugars and lignin to aromatic compounds. The fermentable sugars can further be fermented to ethanol or other products. When the transgenic plants are harvested, the plants are ground to release the cellulase, hemicellulase, and/or ligninase enzymes which then can be used to degrade the lignin and cellulose of the transgenic plants or other plants to produce the fermentable sugars.

Therefore, the present invention provides a transgenic plant capable of expressing one or more cell wall degrading enzymes comprising: at least one DNA comprising a cell wall degrading enzyme coding region operably linked to a nucleotide sequence encoding a signal peptide directing the cell wall degrading enzyme encoded by the DNA to an apoplast, plastid or vacuole of the transgenic plant; and at least one DNA comprising a flowering locus c gene coding region operably linked to a constitutive promoter, wherein the transgenic plant expresses the one or more cell wall degrading enzymes and a transcription factor encoded by the flowering locus c gene that delays flowering while increasing biomass and enabling isolation of increased amounts of the hydrolyzing enzyme from the transgenic plant as compared to a non-transgenic plant from which the transgenic plant is derived.

In further embodiments, the transgenic plant is a monocot. In further embodiments, the monocot is switchgrass and other perennial grasses, rice or maize. In further embodiments, the one or more cell wall degrading enzymes are selected from the group consisting of a cellulase, a hemicellulase and a ligninase. In further embodiments, the cellulase is an endoglucanase, an exoglucanase or a β-glucosidase. In further embodiments, the DNA encoding the cellulase is selected from the group consisting of an e1 gene from Acidothermus cellulyticus, a cbh1 gene from Trichoderma reesei, a dextranase gene from Streptococcus salivarius, and a β-glucosidase gene from Actinomyces naeslundi. In further embodiments, the e1 gene comprises the nucleotide sequence set forth in SEQ ID NO:4, the cbh1 gene comprises the nucleotide sequence set forth in SEQ ID NO:10, the dextranase gene comprises the nucleotide sequence set forth in SEQ ID NO:8, and the β-glucosidase gene comprises the nucleotide sequence set forth in SEQ ID NO:6. In further embodiments, the DNA encodes a β-glucosidase from Butyrivibrio fibrisolvens. In further embodiments, the DNA encoding the β-glucosidase comprises the nucleotide sequence set forth in SEQ ID NO:23. In further embodiments, the DNA encodes a ligninase from Phanerochaete chrysosporium. In still further embodiments, the DNA encoding the ligninase is ckg4 comprising the nucleotide sequence set forth in SEQ ID NO:11 or ckg5 comprising the nucleotide sequence set forth in SEQ ID NO:13. In still further embodiments, the DNA encodes a xylanase from Cochliobolus carbonum. In further embodiments, the DNA encoding the xylanase comprises the nucleotide sequence set forth in SEQ ID NO:24, SEQ ID NO:33, SEQ ID NO:34 or SEQ ID NO:35. In further still embodiments, the transgenic plant further comprises at least one DNA encoding a selectable marker operably linked to a constitutive promoter.

In further still embodiments, the at least one DNA comprising a cell wall degrading enzyme coding region is operably linked to a leaf-specific promoter. In further still embodiments, the leaf-specific promoter is a promoter for rbcS. In further still embodiments, the at least one DNA comprising a cell wall degrading enzyme coding region is operably linked to a Cauliflower Mosaic Virus 35S promoter. In further still embodiments, the at least one DNA comprising a cell wall degrading enzyme coding region is operably linked to a Tobacco Mosaic Virus Ω translational enhancer. In further still embodiments, the nucleotide sequence encoding the signal peptide encodes a signal peptide of rbcS. In further still embodiments, the rbcS comprises the nucleotide sequence set forth in SEQ ID NO:1. In further still embodiments, the nucleotide sequence encoding the signal peptide encodes a signal peptide of tobacco pathogenesis-related protein 1a (Pr1a). In further still embodiments, the transgenic plant further comprises at least one DNA encoding a selectable marker operably linked to a constitutive promoter. In further embodiments, the DNA encoding the selectable marker provides the transgenic plant with resistance to an antibiotic, an herbicide, or to environmental stress. In further still embodiments, wherein the DNA encoding resistance to the herbicide is a DNA encoding phosphinothricin acetyl transferase which confers resistance to the herbicide phosphinothricin.

The present invention provides a transgenic plant capable of expressing cell wall degrading enzymes comprising: at least one DNA encoding a β-glucosidase as a first cell wall degrading enzyme which is operably linked to a nucleotide sequence encoding a signal peptide directing the β-glucosidase to an apoplast, plastid or vacuole of the transgenic plant; at least one DNA encoding a ligninase as a second cell wall degrading enzyme which is operably linked to a nucleotide sequence encoding a signal peptide directing the ligninase to an apoplast, plastid or vacuole of the transgenic plant, at least one DNA encoding a xylanase as a third cell wall degrading enzyme which is operably linked to a nucleotide sequence encoding a signal peptide directing the xylanase to an apoplast, plastid or vacuole of the transgenic plant; and at least one DNA comprising a flowering locus c gene coding region operably linked to a constitutive promoter, wherein the transgenic plant expresses the cell wall degrading enzymes and a transcription factor encoded by the flowering locus c gene that delays flowering while increasing biomass and enabling isolation of increased amounts of the cell wall degrading enzymes from the transgenic plant as compared to a non-transgenic plant from which the transgenic plant is derived.

In further embodiments, the transgenic plant further comprises at least one DNA encoding a selectable marker operably linked to a constitutive promoter. In further embodiments, the DNA encoding the selectable marker provides the transgenic plant with resistance to an antibiotic, an herbicide, or to environmental stress. In further embodiments, the DNA encoding resistance to the herbicide is a DNA encoding phosphinothricin acetyl transferase which confers resistance to the herbicide phosphinothricin. In still further embodiments, the DNA encoding the β-glucosidase is from Butyrivibrio fibrisolvens. In further still embodiments, the DNA encoding the β-glucosidase comprises the nucleotide sequence set forth in SEQ ID NO:23. In further embodiments, the DNA encoding the ligninase is from Phanerochaete chrysosporium. In still further embodiments, the DNA encoding the ligninase is ckg4 comprising the nucleotide sequence set forth in SEQ ID NO:11 or ckg5 comprising the nucleotide sequence set forth in SEQ ID NO:13. In further embodiments, the DNA encoding the xylanase encodes an endoxylanase from Cochliobolus carbonum. In further embodiments, the DNA encoding the xylanase comprises the nucleotide sequence set forth in SEQ ID NO:24, SEQ ID NO:33, SEQ ID NO:34 or SEQ ID NO:35.

The present invention provides a method for making an enzyme extract comprising one or more cell wall degrading enzymes comprising: providing a transgenic plant capable of expressing one or more cell wall degrading enzymes comprising at least one DNA comprising a cell wall degrading enzyme coding region operably linked to a nucleotide sequence encoding a signal peptide directing the cell wall degrading enzyme encoded by the DNA to an apoplast, plastid or vacuole of the transgenic plant; and at least one DNA comprising a flowering locus c gene coding region operably linked to a constitutive promoter, wherein the transgenic plant expresses the one or more cell wall degrading enzymes and a transcription factor encoded by the flowering locus c gene that delays flowering while increasing biomass and enabling isolation of increased amounts of the hydrolyzing enzyme from the transgenic plant as compared to a non-transgenic plant from which the transgenic plant is derived; growing the transgenic plant for a time to accumulate the one or more cell wall degrading enzymes; harvesting the transgenic plant which has accumulated the one or more cell wall degrading enzymes; grinding the transgenic plant to provide an enzyme extract comprising the one or more cell wall degrading enzymes that accumulated in the transgenic plant.

The present invention provides a method for converting lignocellulosic material to fermentable sugars comprising: providing a transgenic plant capable of expressing one or more cell wall degrading enzymes comprising at least one DNA encoding the one or more cell wall degrading enzymes which is operably linked to a nucleotide sequence encoding a signal peptide directing a cellulose, lignin or lignocellulose hydrolyzing enzyme encoded by the DNA to an apoplast, plastid or vacuole of the transgenic plant; and at least one DNA comprising a flowering locus c gene coding region operably linked to a constitutive promoter, wherein the transgenic plant expresses the one or more cell wall degrading enzymes and a transcription factor encoded by the flowering locus c gene that delays flowering while increasing biomass and enabling isolation of increased amounts of the hydrolyzing enzyme from the transgenic plant as compared to a non-transgenic plant from which the transgenic plant is derived; growing the transgenic plant for a time sufficient for the transgenic plant to accumulate the one or more cell wall degrading enzymes; harvesting the transgenic plant which has accumulated the one or more cell wall degrading enzymes; grinding the transgenic plant to provide an enzyme extract comprising the one or more cell wall degrading enzymes that accumulated in the transgenic plant; incubating the lignocellulosic material in the enzyme extract to produce the fermentable sugars from the lignocellulose in the plant material; and extracting the fermentable sugars produced from the lignocellulosic material.

### BRIEF DESCRIPTION OF THE DRAWINGS

FIG. 1 is a diagram of a plasmid containing a heterologous gene expression cassette containing cbh1 operably linked to the rbcS promoter and DNA encoding the rbcS signal peptide and a heterologous gene expression cassette containing the bar gene operably linked to the Act1 promoter. rbcSP is the rbcS gene promoter, SP is DNA encoding the rbcS signal peptide, pin3′ is the 3′ untranslated region of the potato inhibitor II-chloramphenicol acetyltransferase gene, Act1 is the promoter for the act1 gene, and nos is the 3′ untranslated region of the Agrobacterium nopaline synthase gene.

FIG. 2 is a diagram of a plasmid containing a heterologous gene expression cassette containing e1 operably linked to the rbcS promoter and DNA encoding the rbcS signal peptide and a heterologous gene expression cassette containing the bar gene operably linked to the Act1 promoter. The terms in the diagram are as in FIG. 1.

FIG. 3 is a diagram of a heterologous gene expression cassette containing the bar gene in plasmid pDM302. Act1 is the promoter for the act1 gene and nos is the 3′ untranslated region of the Agrobacterium nopaline synthase gene.

FIG. 4 is a diagram of plasmid pSMF13 which is plasmid pSK containing a heterologous gene expression cassette containing cbh1 operably linked to the rbcS promoter. The terms in the diagram are as in FIG. 1.

FIG. 5 is a diagram of plasmid pMSF14 which is plasmid pSK containing a heterologous gene expression cassette containing cbh1 operably linked to the rbcS promoter and DNA encoding the rbcS signal peptide. The terms in the diagram are as in FIG. 1.

FIG. 6 is a diagram of plasmid pMSF15 which is plasmid pBI221 containing a heterologous gene expression cassette containing syn-cbh1 operably linked to the rbcS promoter and DNA encoding the rbcS signal peptide. The terms in the diagram are as in FIG. 1.

FIG. 7 is a diagram of plasmid pTZA8 which is plasmid pBI121 containing a heterologous gene expression cassette containing e1 operably linked to the CaMV35S promoter and DNA encoding the SSU signal peptide. SSU is the glycine max (soybean) rbcS signal peptide. CaMV35S is the cauliflower mosaic virus 35S promoter. The remainder of the terms are as in the diagram are as in FIG. 1.

FIG. 8 is a diagram of plasmid pZA9 which is plasmid pBI121 containing a heterologous gene expression cassette containing e1 operably linked to the CaMV35S promoter and DNA encoding the VSP signal peptide. VSP is the soybean vegetative storage protein beta-leader sequences. The remainder of the terms in the diagram are as in FIG. 7.

FIG. 9 is a diagram of plasmid pZA10 which is plasmid pBI121 containing a heterologous gene expression cassette containing e1 operably linked to the CaMV35S promoter. The remainder of the terms in the diagram are as in FIG. 7.

FIG. 10 is a diagram of a plasmid containing a heterologous gene expression cassette containing ckg4 operably linked to the rbcS promoter and DNA encoding the rbcS signal peptide and a gene expression cassette containing the bar gene operably linked to the Act1 promoter. The remainder of the terms in the diagram are as in FIG. 1.

FIG. 11 is a diagram of a plasmid containing a heterologous gene expression cassette containing ckg5 operably linked to the rbcS promoter and DNA encoding the rbcS signal peptide and a gene expression cassette containing the bar gene operably linked to the Act1 promoter. The remainder of the terms in the diagram are as in FIG. 1.

FIG. 12 is a diagram of plasmid pSMF18 containing a heterologous gene expression cassette containing ckg4 operably linked to the rbcS promoter. The remainder of the terms in the diagram are as in FIG. 1.

FIG. 13 is a diagram of plasmid pSMF19 containing a heterologous gene expression cassette containing ckg5 operably linked to the rbcS promoter. The remainder of the terms in the diagram are as in FIG. 1.

FIG. 14 is a diagram of plasmid pSMF16 containing a heterologous gene expression cassette containing ckg4 operably linked to the rbcS promoter and DNA encoding the rbcS signal peptide. The remainder of the terms in the diagram are as in FIG. 1.

FIG. 15 is a diagram of plasmid pSMF17 containing a heterologous gene expression cassette containing ckg5 operably linked to the rbcS promoter and DNA encoding the rbcS signal peptide. The remainder of the terms in the diagram are as in FIG. 1.

FIG. 16 is an RNA gel blot analysis of FLC in T0 and T1 tobacco plants. Lanes 1-5 are transgenic lines; Lane C is a negative control.

FIG. 17 illustrates FLC transgenic tobacco plants delayed flowering two (2) or more weeks.

FIG. 17A: Right plant is FLC transgenic and left plant is untransformed control. FIG. 17B: Plants from Line 4 (right) compared to control plants (left). FIG. 17C: FLC transgenic versus control flowers from the same age. (A=anther, S=stigma). Note the pollen grains on control anthers and stigma.

FIG. 18 is a restriction map of the plasmid pGreen. RB=T-DNA right border; LB=T-DNA left border; FLC=FLC coding region (0.59 kb); 35S=CaMV 35S promoter; bar=phosphinothricin acetyltransferase gene; Nos=nopaline synthase terminator. Plasmid size: about 6 kb.

FIG. 19 is an RNA-blot analysis of FLC in E1cd-FLC transgenic plants. Lanes: 1 to 6 transgenic lines; C, E1cd control.

FIG. 20 is an E1cd-FLC transgenic tobacco (line 1) plant (right) compared to control E1cd plant (left). Note the short stem and larger leaves of transgenic plant.

FIG. 21 is a schematic representation of ApoE1 binary vector containing the Acedothermus cellulolyticus E1 catalytic domain driven by Cauliflower Mosaic Virus 35S Promoter (CaMV 35S), tobacco Mosaic Virus translational enhancer (Ω), and the sequence encoding the tobacco pathogenesis-related protein 1a (Pr1a) signal peptide for apoplast-targeting of the E1 enzyme, and the polyadenylation signal of nopaline synthase (nos).

FIG. 22A illustrates Gus expression in plantlets of transgenic rice as compared to the untransformed control. FIG. 22B illustrates greenhouse grown A. cellulolyticus E1 transgenic rice plants.

FIGS. 23A-D illustrate a PCR (A), Southern (B), Northern (C) and Western (D) Blot analysis that show the presence of the transgenes in five transgenic rice lines. FIG. 23A illustrates Left; PCR amplification of the bar (0.59 kb), and right; E1 (1 kb) (b) genes in 5 transgenic rice lines. M: Ladder marker 100 bp (a) and 1 kb (b), P: Plasmid (positive control), C: Non-transformed (negative control), 1-5: Transgenic lines. FIG. 23B illustrates a Southern blot analysis for E1 transgene showing different bands for the five transgenic rice lines. P: plasmid; C: non-transgenic control; 1-5: Transgenic lines. FIG. 23C illustrates a Northern blot analysis showing 1 kb bands for the five transgenic rice lines. +: positive control; C: non-transgenic control; 1-5: Transgenic lines. FIG. 23D illustrates a Western blot analysis showing 40 kDa bands for the five transgenic rice lines. +: positive control; C: non-transgenic control; 1-5: Transgenic lines.

FIG. 24A and FIG. 24B illustrate immunofluorescence confocal microscopy for the transgenic (FIG. 24A) and untransformed (FIG. 24B) rice showing apoplast localization of the E1 enzyme in transgenic rice leaves.

FIG. 25 shows the detection of the E1 enzyme activity using CMCase activity assay. Zones of CMC hydrolysis were decolorized with washing leaving yellow regions in the transgenic as compared to red background in the control.

FIG. 26 illustrates in FIG. 26A the amount of glucose released from the enzymatic hydrolysis of CMC (1%, 5%, 10%) and Avicel (1%, 5%, 10%) using total protein extracted from E1 expressed rice straw. In FIG. 26B is shown the comparison of percentage of glucan converted in the enzymatic hydrolysis of corn stover (CS) and rice straw (RS). CE, commercial enzyme, UT, untreated biomass, CS1, RS1, CS2, and RS2 represent, reaction done using 0.5 ml and 4 ml of total protein (with 4.9% of E1) and commercial β-glucosidase (6.5 mg/15 ml) respectively.

FIG. 27 is a schematic drawing of the plasmid pMZ766 used to produce transgenic maize plants. A 1.076-kb Sac I restriction fragment was used as the probe for Southern blot analysis.

FIG. 28 is a PCR analysis of the DNA from plants recovered after transformation with the pMZ766. Lanes 1, untransformed maize control; 2-6, five independent transgenic maize plants; 7, plasmid control, and 8, 1-kb plus DNA ladder.

FIG. 29 is a Southern blot analysis of genomic DNA from maize plants, probed with the E1-cd. Lane 1, 10 pg of Sac I digest E1-cd fragment from p MZ766; Lanes 2-3, untransformed maize control (lane 2; DNA undigested and lane 3; DNA digested); Lanes 4-13; Five independent pMZ766 transformants; (4, 6, 8, 10, 12) DNA not digested; (5, 7, 9, 11, 13) DNA digested with Sac I. Size of bands is 1 kb.

FIG. 30 is a Western blot of total soluble protein from transgenic maize plants expressing E1-cd. Lanes +C, positive tobacco control; −C, untransformed maize control; 1-6; transgenic maize plants.

FIG. 31 illustrates a schematic representation of plasmid vectors containing two cassettes, one containing the Acedothermus cellulolyticus E1 catalytic domain or the ligninase (CGL4) driven by maize rubisco promoter (rbcS) or Cauliflower Mosaic Virus 35S Promoter (CaMV 35S), tobacco Mosaic Virus translational enhancer (Ω), and the sequence encoding the tobacco pathogenesis-related protein 1a (Pr1a) signal peptide for apoplast-targeting or the maize rbcs signal peptide for enzyme targeting, and the polyadenylation signal of nopaline synthase (Nos).

FIG. 32 is an illustration of a plasmid used in a 1:1 ratio with the plasmid of FIG. 27 in maize transformation experiments. In the two constructs, Ω represents for the tobacco Mosaic Virus translational enhancer, Pr1a SP for the sequence encoding the tobacco pathogenesis-related protein 1a signal peptide for apoplast-targeting of the E1 enzyme, Nos for the polyadenylation signal of nopaline synthase, and bar for the herbicide resistance sequences.

FIGS. 33A and B illustrates immunofluorescent confocal laser microscopy of apoplast-targeted E1 transgenic maize leaf tissue (FIG. 33A) using the E1 primary antibody and the FITC anti-mouse secondary antibody. FIG. 33B illustrates immunofluorescent confocal laser microscopy of leaf tissue from an untransformed control maize plant.

FIG. 34 illustrates biological activity and conversion ability of maize-produced E against corn stover (CS), Avaeil and CMC. As was performed in our rice work, commercial β-glucosidase (6.5 mg/15 ml) was added to convert cellubiose into glucose.

FIG. 35 is immunofluorescent localization of PHBC. Immunofluorescent confocal laser microscopy of choloroplast-targeted polyhydroxybutyrate C in transgenic maize leaf tissue (left) using the PHBC primary antibody and the FITC secondary antibody. Photo on the right is leaf tissue from an untransformed control maize plant.

FIG. 36 shows corn GFP-chloroplast embryos.

FIG. 37 is a Western blot of E1 transgenic maize as compared to E1 transgenic rice and tobacco plants produced in the inventor's laboratory. Lane “+”=Transgenic tobacco as positive control. Lane “−C”=Maize control (untransformed). Lanes 1 to 10 represent at least 5 different transformation events. Lane 11=E1 Transgenic rice as another positive control

FIG. 38 is a schematic of a plasmid containing the Butyrivibrio fibrisolvens β-glucosidase (Yao, 2004) cDNA regulated by the 35S promoter and enhancer. This construct too contains the sequences encoding the tobacco pathogenesis-related protein 1a (Pr1a) signal peptide for targeting of β-glucosidase enzyme into plant apoplast.

FIG. 39 is a schematic of a plasmid containing the xylanase cDNA regulated by the 35S promoter and enhancer. This construct contains the sequences encoding the tobacco pathogenesis-related protein 1a (Pr1a) signal peptide for targeting of Cochliobolus carbonum endoxylanase (Apel et al., Mol. Plant-Microbe Interact, 6:467-473 (1993)) into plant apoplast.

FIG. 40 is a schematic of a plasmid containing the Phanerochaete chrysosporium ligninase (de Boer et al., 1988) gene regulated by the 35S promoter and enhancer.

FIG. 41 is a schematic of a more detailed map of plasmid pGreen, which has the Arabidopsis Flowering Locus C (FLC) coding sequences regulated by 35S promoter and Nos terminator. The bar herbicide resistance selectable marker is regulated by 35S promoter and Nos terminator. The sizes of each are shown in kilobases (kb).

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The term “cell wall degrading enzyme” as used herein refers to any cellulase, hemicellulase or ligninase.

The term “cellulase” as used herein is a generic term that includes endoglucanases, exoglucanases and β-glucosidases. The term includes endoglucanases such as the E1 beta-1,4-endoglucanase precursor gene (e1) of Acidothermus cellulolyticus and exoglucanases such as the cellobiohydrolase gene (cbh1) of Trichoderma reesei (also classified by some as Trichoderma longibrachiatum), the dextranase gene of Streptococcus salivarius encoding the 1,6-alpha-glucanhydrolase gene, and the β-glucosidase gene from Butyrivibrio fibrisolvens or Actinomyces naeslundi. Endoglucanases randomly cleave cellulose chains into smaller units. Exoglucanases include cellobiohydrolases, which liberate glucose dimers (cellobiose) from the ends of cellulose chains; glucanhydrolases, which liberate glucose monomers from the ends of cellulose chains; and, β-glucosidases, which liberate D-glucose from cellobiose dimers and soluble cellodextrins. When all four of the above enzymes are combined, they work synergistically to rapidly decrystallize and hydrolyze cellulose to fermentable sugars.

The term “hemicellulase” as used herein is a generic term which encompasses all varieties of enzymes that degrade any type of hemicellulose such as xylan, glucuronoxylan, arabinoxylan, glucomannan and xyloglucan. Some examples include, but are not limited to xylanase. Examples of beta-1,4-xylanase genes include XYL1 (SEQ ID NO:24), and XYL2 (SEQ ID NO:33), XYL3 (SEQ ID NO:34) and XYL4 (SEQ ID NO:35) of Cochliobolus carbonum as described by Apel-Birkhold et al., Applied and Environmental Microbiology, November 1996, pp. 4129-4135. Further examples of xylanases include those described in U.S. Pat. No. 6,682,923 to Bentzien et al., hereby incorporated herein by reference in its entirety.

The term “lignin” is used herein as a generic term that includes both lignins and lignocelluloses.

The term “ligninase” is used herein as a generic term that includes all varieties of enzymes which degrade lignins such as the lignin peroxidase gene of Phanerochaete chrysosporium. Ligninase enzymes degrade lignin into phenolics, unlike cellulases that hydrolyse cellulase into sugars. Ligninase can be provided in the transgenic plants of the present invention so that after harvesting and grinding the plants, ligninase will remove lignin of the lignicellulosic matter for better access of cellulases to the cellulose in the plant matter. Thus, the present invention reduces or eliminates the need for expensive heat and/or chemical pretreatments to remove lignin.

The term “monocot” as used herein refers to all monocotyledonae plants including, but not limited to cereal plants such as maize, wheat, barley, oats, rye, rice, buckwheat, millet, and sorghum. Additionally monocot plants include switchgrass, and other perennial grasses. Other monocots include such plants as sugar cane.

The term “Pr1a” as used herein refers to tobacco pathogenesis-related protein 1a signal peptide encoding a sequence for apoplast-targeting of proteins. U.S. Pat. No. 6,750,381 to Mitsuhara et al., hereby incorporated herein by reference in its entirety, teaches the Pr1a signal peptide.

A variety of fungi and bacteria produce ligninase and cellulase enzymes, and based on evolutionary pressures, these fungi are able to degrade lignin or cellulose and hemicellulose of plant residues in the soil. In the laboratory, cellulases have been used to hydrolyze or convert cellulose and hemicellulose into mixtures of simple sugars that can be used in fermentation to produce a wide variety of useful chemical and fuel products, including but not limited to, ethanol, lactic acid, and 1,3-propanediol, which is an important molecular building block in the production of environmentally-friendly plastics.

The biodegradation of lignin, which comprises 20-30% of the dry mass of woody plants, is of great economic importance because this process is believed to be an important rate-limiting step in the earth's carbon cycle. Furthermore, there is considerable potential for the transformation of lignin into aromatic chemical feedstock. Also, delignification of lignocellulosic feeds has been shown to increase their digestibility by cattle by about 30%, therefore, contributing to enhanced cost effectiveness for producing milk and meat. Moreover, research on lignin biodegradation has important implications in biopulping and biobleaching in the paper industry.

The present invention provides transgenic plants which produce ligninases, cellulases, or both in the leaves and straw/stalks of the plant. While the transgenic plant can be any plant which is practical for commercial production, it is preferable that the transgenic plants be constructed from plants which are produced in large quantities and which after processing produce a substantial amount of leaves and stalks as a byproduct. Therefore, it is desirable that the transgenic plant be constructed from plants including, but not limited to, maize, wheat, barley, rye, hops, hemp, rice, potato, soybean, sorghum, sugarcane, clover, tobacco, alfalfa, arabidopsis, coniferous trees, and deciduous trees. Most preferably, the transgenic plant is constructed from maize.

Maize is a preferred plant because it is a major crop in the United States; approximately 60 million acres of maize are produced per year. Further, there is already a large industry built around the processing of maize grain to industrial products, which includes the production of over 1.2 billion gallons of fuel ethanol per year. Thus, fermentable sugars produced by the hydrolysis of maize stalks and leaves according to the present invention can be utilized within the large existing maize refining infrastructure. Leaves and stalks from transgenic maize made according to the present invention can be made available to this refining infrastructure in large quantities, about tens of millions of tons annually) at a current cost of about 30 dollars per ton. This cost is about one quarter of the cost of maize grain which further enhances the value of the present invention for the economical production of a wide variety of industrial products from the residue of transgenic plants made according to the present invention. Furthermore, maize is preferred because it is a C-4 monocot that has very large chloroplasts. The large chloroplasts enables the chloroplasts of the transgenic maize of the present invention to accumulate higher levels of ligninases and cellulases than could be accumulated in the chloroplasts of other transgenic plants, e.g., C-3 dicots and monocots. Therefore, transgenic maize of the present invention is a particularly useful source of ligninases and cellulases.

Thus, the transgenic plants of the present invention provide a plentiful, inexpensive source of fungal or bacterial ligninases and cellulases which can be used to degrade lignins and cellulose in plants to fermentable sugars for production of ethanol or for other uses which require ligninases and cellulases such as pre-treating silage to increase the energy value of lignocellulosic feeds for cows and other ruminant animals, pre-treating lignocellulosic biomass for fermentative conversion to fuels and industrial chemicals, and biodegradation of chloroaromatic environmental pollutants. Because the transgenic plants of the present invention produce the ligninases, cellulases, or both therein, the external addition of ligninases and cellulases for degradation of the plant material is no longer necessary. Therefore, the present invention enables the plant biomass, which is destined to become ethanol or other products, to serve as the source of ligninase and cellulase. Furthermore, the plant material from the transgenic plants of the present invention can be mixed with non-transgenic plant material. The ligninases, cellulases, or both produced by the transgenic plants will degrade the lignin and cellulose of all the plant material, including the non-transgenic plant material. Thus, ligninase and cellulase degradation of plant material can be carried out more economically.

The transgenic plants of the present invention comprise one or more heterologous gene expression cassettes containing DNA encoding at least one fungal or bacterial ligninase, cellulase, or both inserted into the plant's nuclear genome. The preferred cellulase is encoded by a DNA from the microorganism Acidothermus cellulolyticus, Thermomonospora fusca, and Trichoderma reesei (Trichoderma longibrachiatum). Other microorganisms which produce cellulases suitable for the present invention include Zymomonas mobilis, Acidothermus cellulolyticus, Cloostridium thermocellum, Eiwinia chrysanthemi, Xanthomonas campestris, Alkalophilic Baccilus sp., Cellulomonas fimi, wheat straw mushroom (Agaricus bisporus), Ruminococcus flavefaciens, Ruminococcus albus, Fibrobacter succinogenes, and Butyrivibrio fibrisolvens.

The Preferred Ligninase is Lignin peroxidase (LIP) encoded in DNA from Phanerochaete chrysosporium or Phlebia radiata. One of the major lignin-degrading, extracellular enzymes produced by P. chrysosporium is LIP. The LIPs are glycosylated heme proteins (MW 38 to 46 kDa) which are dependent on hydrogen peroxide for activity and catalyze the oxidative cleavage of lignin polymer. At least six heme proteins (H1, H2, H6, H7, H8, and H10) with LIP activity have been identified in P. chrysosporium strain BKMF-1767 of which isozymes H2, H6, H8, and H10 are the major LIPs in both static and agitated cultures of P. chrysosporium. However, other fungi which produce ligninases suitable for use in the present invention include Bjerkandera adusta, Trametes hirsuta, Plebia radiata, Pleurotus spp., Stropharia aurantiaca, Hypholoma fasciculare, Trametes versicolor, Gymnopilus penetrnas, Stereum hirsutum, Mycena haematopus, and Armillaria mellea.

In the present invention, the transgenic plant comprises a DNA encoding one or more cellulase fusion proteins wherein the DNA encoding the cellulases are operably linked to a DNA encoding a signal peptide which directs the cellulase fusion protein to a plant organelle such as the nucleus, a microbody (e.g., a peroxisome, or specialized version thereof, such as a glyoxysome), an endoplasmic reticulum, an endosome, a vacuole, a mitochondria, a chloroplast, or a plastid. By sequestering the cellulase fusion proteins in the plant organelle, the cellulase fusion protein is prevented from leaking outside the cytoplasm to harm the plant by degrading the cellulose in the plant's cell wall while the plant is being cultivated. In particular embodiments of the present invention, the gene encoding the cellulase is modified by replacing the amino acid codons that encode the leader region of the cellulase with amino acid codons that encode the signal peptide.

In a preferred embodiment of the invention, the amino acid codons that encode the signal peptide that directs the protein to which it is attached to the plant organelle, the chloroplasts, are the nucleotide codons that encode the rice rubisco synthase gene (rbcS) small subunit signal peptide (rbcSSP). The nucleotide sequence of the rbcS is set forth in SEQ ID NO:1 (GenBank Accession No. X07515). The 47 amino acid signal peptide of the rbcS protein has the amino acid sequence MAPPS VMASS ATIVA PFQGS SPPPA CRRPP SELQL RQRQH GGRIR CM (SEQ ID NO:2). The rbcS SP directs proteins to which it is operably linked to the chloroplasts of the transgenic plant. Therefore, in the preferred embodiment of the present invention, the transgenic plant comprises a DNA encoding the cellulase operably linked with a DNA encoding the rbcS SP to produce the cellulase fusion protein. The rbcS SP directs the cellulase fusion protein to the chloroplasts. Thus, the cellulase fusion protein produced by the transgenic plant accumulates in the chloroplasts of the transgenic plant which protects the transgenic plant from degradation by the cellulase fusion protein while it is being cultivated. Alternatively, the DNA encoding the cellulase is modified at its 3′end to encode a transit peptide such as the peptide RAVARL (SEQ ID NO:3), which targets the ligninase fusion protein to the peroxisomes (U.S. Pat. No. 6,103,956 to Srienc et al.). Preferably, the leader region of the cellulase is also removed. In any one of the above embodiments, the cellulase can be further modified to include a GC content that approximates the GC content of the genomic DNA of the plant by methods well known in the art.

In a preferred embodiment, the cellulase comprising the cellulase fusion protein is encoded by the EI beta-1,4-endoglucanase precursor gene (e1) of Acidothermus cellulolyticus, the cellobiohydrolase gene (cbh1) of Trichoderma reesei (Trichoderma longibrachiatum), the beta-glucosidase gene from Actinomyces naeslundi, or the glucanhydrolase (dextranase) gene from Streptococcus salivarius. The nucleotide sequence of the e1 DNA is set forth in SEQ ID NO:4 (GenBank Accession No. U33212), which encodes the cellulase with the amino acid sequence set forth in SEQ ID NO:5. SEQ ID NO:6 provides the nucleotide sequence of the beta-glucosidase gene from Actinomyces naeslundi (GenBank Accession No. AY029505), which encodes the beta-glucosidase with the amino acid sequence set forth in SEQ ID NO:7. SEQ ID NO:8 provides the nucleotide sequence of the dextranase gene from Streptococcus salivarius (GenBank Accession No. D29644), which encodes a glucanhydrolase with the amino acid sequence set forth in SEQ ID NO:9. The nucleotide sequence of cbh1 is set forth in SEQ ID NO:10 (GenBank Accession No. E00389), which encodes the cellulase that includes the joined exons from positions 210 to 261, 738 to 1434, and 1498-1881.

In the present invention, the transgenic plant comprises a DNA encoding one or more ligninase fusion proteins wherein a DNA encoding the ligninase is operably linked to a DNA encoding a signal peptide which directs the ligninase fusion protein to a plant organelle. By sequestering the ligninase fusion proteins in the plant organelles, the modified ligninase is prevented from leaking outside the cytoplasm to harm the plant by degrading the ligninase in the plant's cell wall while the plant is being cultivated. In particular embodiments of the present invention, the leader sequence of the gene encoding the ligninase is modified by replacing the amino acid codons that encode the leader region of the ligninase with amino acid codons that encode the signal peptide.

In a preferred embodiment of the invention, the amino acid codons that encode the signal peptide are the amino acid codons which encode the rice rubisco synthase gene (rbcS) small subunit signal peptide (rbcSSP). The nucleotide sequence of the rbcS is set forth in SEQ ID NO:1 (GenBank Accession No. X07515). The 47 amino acid signal peptide of the rbcS protein has the amino acid sequence MAPPS VMASS ATIVA PFQGS SPPPA CRRPP SELQL RQRQH GGRIR CM (SEQ ID NO:2). Therefore, in the preferred embodiment of the present invention, the transgenic plant comprises a DNA encoding the ligninase operably linked to a DNA encoding the rbcS SP. The rbcS SP directs the ligninase fusion protein to the chloroplasts. Thus, the ligninase fusion protein produced by the transgenic plant accumulates in the chloroplasts of the transgenic plant which protects the transgenic plant from degradation by the ligninase fusion protein while it is being cultivated. Alternatively, the DNA encoding the ligninase is modified at its 3′end to encode a transit peptide such as the peptide RAVARL (SEQ ID NO:3). Optionally, the leader region of the ligninase is also removed. In any one of the above embodiments, the ligninase can be further modified to include a GC content that approximates the GC content of the genomic DNA of the plant by methods well known in the art.

In a preferred embodiment of the invention, the ligninase comprising the ligninase fusion protein is encoded by the lignin peroxidase gene (LIP) genes ckg4 (H2) and ckg5 (H10) of Phanerochaete crysosporium (de Boer et al., Gene 60: 93-102 (1987), Corrigendum in Gene 69: 369 (1988)). The nucleotide sequence of the ckg4 gene is set forth in SEQ ID NO:11 (GenBank Accession No. M18743), which encodes the amino acid with the sequence set forth in SEQ ID NO:12. The nucleotide sequence of the ckg5 gene is set forth in SEQ ID NO:13 (GenBank Accession No. M18794), which encodes the amino acid with the sequence set forth in SEQ ID NO:14.

In the present invention, transcription and, therefore, expression of the ligninase and cellulase fusion proteins are effected by a promoter that is active in a particular tissue of the plant, e.g., a promoter that is active primarily in the leaves of a plant. A leaf-specific promoter that is preferred for transcription (expression at the RNA level) is the rice rubisco synthase gene promoter (rbcSP), which has the nucleotide sequence prior to the rbcS gene coding region included in SEQ ID NO:1. In some embodiments of the present invention, it is desirable to relegate transcription of the heterologous gene expression cassette to the seeds using a seed-specific promoter. Seed-specific promoters that are suitable include, but are not limited to, the seed-specific promoters such as the maize 19 kDa zein (cZ19B1) promoter, the maize cytokinin-induced message (Cim1) promoter, and the maize myo-inositol-1-phosphate synthase (milps) promoter, which are disclosed in U.S. Pat. No. 6,225,529 to Lappegard et al. Therefore, in the heterologous gene expression cassettes, the nucleotide sequence comprising rbcS promoters are operably linked to the nucleotide sequences encoding the ligninase and cellulase fusion proteins. Thus, in a transgenic plant of the present invention, transcription of the ligninase and cellulase fusion proteins occurs primarily in the leaves of the plant, and because the ligninase and cellulase fusion proteins each has a signal peptide that directs its transport to plastids, the ligninase and cellulase fusion proteins accumulate in the plastids.

In the preferred embodiment of the present invention, the 3′ ends of the nucleotide sequence encoding the above ligninase and cellulase fusion proteins are operably linked to a 3′ noncoding sequence wherein the noncoding sequence contains a poly(A) cleavage/addition site and other regulatory sequences which enables the RNA transcribed therefrom to be properly processed and polyadenylated which in turn affects stability, transport and translation of the RNA transcribed therefrom in the plant cell. Examples of 3′ noncoding sequences include the 3′ noncoding sequence from the potato protease inhibitor II gene, which includes nucleotides 871 to 1241 of SEQ ID NO:15 (GenBank Accession No. M15186) and the 3′ noncoding sequence from the Agrobacterium nopaline synthase gene, which includes nucleotides 2001 to 2521 of SEQ ID NO:16 (GenBank Accession No. V00087 J01541).

The above heterologous gene expression cassettes can be constructed using conventional molecular biology cloning methods. In a particularly convenient method, PCR is used to produce the nucleotide fragments for constructing the gene expression cassettes. By using the appropriate PCR primers, the precise nucleotide regions of the above DNAs can be amplified to produce nucleotide fragments for cloning. By further including in the PCR primers restriction enzyme cleavage sites which are most convenient for assembling the heterogenous gene expression cassettes (e.g., restriction enzyme sites that are not in the nucleotide fragments to be cloned), the amplified nucleotide fragments are flanked with the convenient restriction enzyme cleavage sites for assembling the nucleotide fragments into heterogenous gene expression cassettes. The amplified nucleotide fragments are assembled into the heterogeneous gene expression cassettes using conventional molecular biology methods. Based upon the nucleotide sequences provided herein, how to construct the heterogenous gene expression cassettes using conventional molecular biology methods with or without PCR would be readily apparent to one skilled in the art.

In a further embodiment of the present invention, the transgenic plant comprises more than one heterogeneous gene expression cassette. For example, the transgenic plant comprises a first cassette which contains a DNA encoding a ligninase fusion protein, and one or more cassettes each containing a DNA encoding a particular cellulase fusion protein. Preferably, both the ligninase and cellulase fusion proteins comprise amino acids of a signal peptide which directs the fusion proteins to plant organelles. In a preferred embodiment, the signal peptide for each is the rbcS SP or the SKL motif.

In a further still embodiment, the transgenic plant comprises DNA encoding the ligninase fusion protein such as the ckg4 or ckg5 LIP, an endoglucanase fusion protein such as the e1 fusion protein, and a cellobiohydrolase fusion protein such as the cbh1 fusion protein. In a further still embodiment, the transgenic plant comprises DNA encoding the ligninase fusion protein such as the ckg4 or ckg5 LIP, an endoglucanase fusion protein such as the e1 fusion protein, a cellobiohydrolase fusion protein such as the cbh1 fusion protein, a beta-glucosidase, and a glucanhydrolase. Preferably, both the ligninase and cellulase fusion proteins comprise amino acids of a signal peptide which directs the fusion proteins to plant organelles. In a preferred embodiment, the signal peptide for each is the rbcS SP or the SKL motif.

To make the transgenic plants of the present invention, plant material such as meristem primordia tissue is transformed with plasmids, each containing a particular heterogenous gene expression cassette using the Biolistic bombardment method as described in Example 5 and in U.S. Pat. No. 5,767,368 to Zhong et al. Further examples of the Biolistic bombardment method are disclosed in U.S. application Ser. No. 08/036,056 and U.S. Pat. No. 5,736,369 to Bowen et al. Each heterogenous gene expression cassette is separately introduced into a plant tissue and the transformed tissue propagated to produce a transgenic plant that contains the particular heterogenous gene expression cassette. Thus, the result is a transgenic plant containing the heterogenous gene expression cassette expressing a ligninase such as ckg4 or ckg5, a transgenic plant containing a heterogenous gene expression cassette expressing endoglucanase such as e1, a transgenic plant containing a heterogenous gene expression cassette expressing a cellobiohydrolase such as cbh1, a transgenic plant containing a heterogenous gene expression cassette expressing an exoglucanase such as beta-glucosidase, and a transgenic plant containing a heterogenous gene expression cassette expressing an exoglucanase such as glucanhydrolase.

Alternatively, transformation of corn plants can be achieved using electroporation or bacterial mediated transformation using a bacterium such as Agrobacterium tumefaciens to mediate the transformation of corn root tissues (see Valvekens et al. Proc. Nat'l. Acad. Sci. USA. 85: 5536-5540 (1988)) or meristem primordia.

In a preferred embodiment of the present invention, the transgenic plant comprises one or more ligninase fusion proteins and one or more cellulase fusion proteins. Construction of the preferred transgenic plant comprises making first generation transgenic plants as above, each comprising a ligninase fusion protein, and transgenic plants as above, each comprising a cellulase fusion protein. After each first generation transgenic plant has been constructed, progeny from each of the first generation transgenic plants are cross-bred by sexual fertilization to produce second generation transgenic plants comprising various combinations of both the ligninase fusion protein and the cellulase fusion protein.

For example, various combinations of progeny from the first generation transgenic plants are cross-bred to produce second generation transgenic plants that contain ckg4 and cbh1, e1, beta-glucosidase, or ckg5; second generation transgenic plants that contain ckg5 and cbh1, e1, or beta-glucosidase; second generation transgenic plants that contain e1 or beta glucosidase, and a second generation transgenic plant that contains e1 and beta-glucosidase.

Progeny of the second generation transgenic plants are cross-bred by sexual fertilization among themselves or with first generation transgenic plants to produce third generation transgenic plants that contain one or more ligninases, one or more cellulases, or combinations thereof.

For example, cross-breeding a second generation transgenic plant containing ckg4 and cbh1 with a second generation transgenic plant containing e1 and beta-glucosidase produces a third generation transgenic plant containing ckg4, cbh1, e1, and beta-glucosidase. The third generation transgenic plant can be cross-bred with a first generation transgenic plant containing ckg5 to produce a fourth generation transgenic plant containing ckg4, ckg5, cbh1, e1, and beta-glucosidase.

It will be readily apparent to one skilled in the art that other transgenic plants with various combinations of ligninases and cellulases can be made by cross-breeding progeny from particular transgenic plants. Zhang et al, Theor. Appl. Genet. 92: 752-761, (1996), Zhong et al, Plant Physiol. 110: 1097-1107, (1996), and Zhong et al, Planta, 187: 483-489, (1992) provide methods for making transgenic plants by sexual fertilization.

Alternatively, plant material is transformed as above with a plasmid containing a heterologous gene expression cassette encoding the ligninase fusion protein. The transgenic plant is recovered from the progeny of the transformed plant material. Next, plant material from the transgenic plant is transformed with a second plasmid containing a heterologous gene expression cassette encoding the cellulase fusion protein and a second selectable marker. The transgenic plant is recovered from the progeny of the transformed plant material. It will be readily apparent to one skilled in the art that transgenic plants containing any combination of ligninases and cellulases can be made by the above method.

In a preferred embodiment, the above heterologous gene expression cassettes further include therein nucleotide sequences that encode one or more selectable markers which enable selection and identification of transgenic plants that express the modified cellulase of the present invention. Preferably, the selectable markers confers additional benefits to the transgenic plant such as herbicide resistence, insect resistance, and/or resistence to environmental stress.

Alternatively, the above transformations are performed by co-transforming the plant material with a first plasmid containing a heterologous gene expression cassette encoding a selectable marker and a second plasmid containing a heterologous gene expression cassette encoding a ligninase or cellulase fusion protein. The advantage of using a separate plasmid is that after transformation, the selectable marker can be removed from the transgenic plant by segregation, which enables the selection method for recovering the transgenic plant to be used for recovering transgenic plants in subsequent transformations with the first transgenic plant.

Examples of preferred markers that provide resistence to herbicides include, but are not limited to, the bar gene from Streptomyces hygroscopicus encoding phosphinothricin acetylase (PAT), which confers resistance to the herbicide glufonsinate; mutant genes which encode resistance to imidazalinone or sulfonylurea such as genes encoding mutant form of the ALS and AHAS enzyme as described by Lee at al. EMBO J. 7: 1241 (1988) and Miki et al., Theor. Appl. Genet. 80: 449 (1990), respectively, and in U.S. Pat. No. 5,773,702 to Penner et al.; genes which confer resistance to glycophosphate such as mutant forms of EPSP synthase and aroA; resistance to L-phosphinothricin such as the glutamine synthetase genes; resistance to glufosinate such as the phosphinothricin acetyl transferase (PAT and bar) gene; and resistance to phenoxy proprionic acids and cycloshexones such as the ACCAse inhibitor-encoding genes (Marshall et al. Theor. Appl. Genet. 83: 435 (1992)). The above list of genes which can import resistance to an herbicide is not inclusive and other genes not enumerated herein but which have the same effect as those above are within the scope of the present invention.

Examples of preferred genes which confer resistance to pests or disease include, but are not limited to, genes encoding a Bacillus thuringiensis protein such as the delta-endotoxin, which is disclosed in U.S. Pat. No. 6,100,456 to Sticklen et al.; genes encoding lectins, (Van Damme et al., Plant Mol. Biol. 24: 825 (1994)); genes encoding vitamin-binding proteins such as avidin and avidin homologs which can be used as larvicides against insect pests; genes encoding protease or amylase inhibitors, such as the rice cysteine proteinase inhibitor (Abe et al., J. Biol. Chem. 262: 16793(1987)) and the tobacco proteinase inhibitor I (Hubb et al., Plant Mol. Biol. 21: 985(1993)); genes encoding insect-specific hormones or pheromones such as ecdysteroid and juvenile hormone, and variants thereof, mimetics based thereon, or an antagonists or agonists thereof; genes encoding insect-specific peptides or neuropeptides which, upon expression, disrupts the physiology of the pest; genes encoding insect-specific venom such as that produced by a wasp, snake, etc.; genes encoding enzymes responsible for the accumulation of monoterpenes, sesquiterpenes, asteroid, hydroxaminc acid, phenylpropanoid derivative or other non-protein molecule with insecticidal activity; genes encoding enzymes involved in the modification of a biologically active molecule (see U.S. Pat. No. 5,539,095 to Sticklen et al., which discloses a chitinase that functions as an anti-fungal); genes encoding peptides which stimulate signal transduction; genes encoding hydrophobic moment peptides such as derivatives of Tachyplesin which inhibit fungal pathogens; genes encoding a membrane permease, a channel former or channel blocker (for example cecropin-beta lytic peptide analog renders transgenic tobacco resistant to Pseudomonas solanacerum)(Jaynes et al. Plant Sci. 89: 43 (1993)); genes encoding a viral invasive protein or complex toxin derived therefrom (viral accumulation of viral coat proteins in transformed cells of some transgenic plants impart resistance to infection by the virus the coat protein was derived as shown by Beachy et al. Ann. Rev. Phytopathol. 28: 451 (1990); genes encoding an insect-specific antibody or antitoxin or a virus-specific antibody (Tavladoraki et al. Nature 366: 469(1993)); and genes encoding a developmental-arrestive protein produced by a plant, pathogen or parasite which prevents disease. The above list of genes which can import resistance to disease or pests is not inclusive and other genes not enumerated herein but which have the same effect as those above are within the scope of the present invention.

Examples of genes which confer resistence to environmental stress include, but are not limited to, mt1d and HVA1, which are genes that confer resistance to environmental stress factors; rd29A and rd19B, which are genes of Arabidopsis thaliana that encode hydrophilic proteins which are induced in response to dehydration, low temperature, salt stress, or exposure to abscisic acid and enable the plant to tolerate the stress (Yamaguchi-Shinozaki et al., Plant Cell 6: 251-264 (1994)). Other genes contemplated can be found in U.S. Pat. Nos. 5,296,462 and 5,356,816 to Thomashow. The above list of genes, which can import resistance to environmental stress, is not inclusive and other genes not enumerated herein but which have the same effect as those above are within the scope of the present invention.

Thus, it is within the scope of the present invention to provide transgenic plants which express one or more ligninase fusion proteins, one or more cellulase fusion proteins, and one or more of any combination of genes which confer resistance to an herbicide, pest, or environmental stress.

In particular embodiments of the present invention, the heterologous gene expression cassettes can further be flanked with DNA containing the matrix attachment region (MAR) sequence. While use of MAR in the present invention is optional, it can used to increase the expression level of transgenes, to get more reproducible results, and to lower the average copy number of the transgene (Allen et al., The Plant Cell 5: 603-613 (1993); Allen et al., The Plant Cell 8: 899-913 (1996); Mlynarova et al., The Plant Cell 8: 1589-1599 (1996)).

To degrade the lignocellulose in the leaves and stalks of the transgenic plants of the present invention, the transgenic plant is ground up to produce a plant material using methods currently available in the art to disrupt a sufficient number of the plant organelles containing the ligninase and cellulase therein. The ligninase and cellulase degrade the lignocellulose of the transgenic plant into fermentable sugars, primarily glucose, and residual solids. The fermentable sugars are used to produce ethanol or other products.

The transgenic plants can be processed to ethanol in an improvement on the separate saccharification and fermentation (SHF) method (Wilke et al., Biotechnol. Bioengin. 6: 155-175 (1976)) or the simultaneous saccharification and fermentation (SSF) method disclosed in U.S. Pat. No. 3,990,944 to Gauss et al. and U.S. Pat. No. 3,990,945 to Huff et al. The SHF and SSF methods require pre-treatment of the plant material feedstock with dilute acid to make the cellulose more accessible followed by enzymatic hydrolysis using exogenous cellulases to produce glucose from the cellulose, which is then fermented by yeast to ethanol. In some variations of the SHF or SSF methods, the plant material is pre-treated with heat or with both heat and dilute acid to make the cellulose more accessible.

An SHF or SSF method that uses the transgenic plant material of the present invention as the feedstock is an improvement over the SHF or SSF method because the transgenic plant material contains its own cellulases and ligninases or cellulases. Therefore, exogenous ligninases and/or cellulases do not need to be added to the feedstock. Furthermore, because particular embodiments of the transgenic plant material produce ligninase, the need for pre-treatment of the plant material in those embodiments before enzymatic degradation is not necessary. In a further improvement over the SHF method, the transgenic plant material is mixed with non-transgenic plant material and the mixture processed to ethanol.

The following examples are intended to promote a further understanding of the present invention.

### EXAMPLE 1

This example shows the construction of plasmids comprising a heterologous gene expression cassette comprising a DNA encoding a cellulase fusion protein and a heterologous gene expression cassette comprising a DNA encoding the bar gene (Table 1).

TABLE 1

| | Construct | Plasmid features |
|---|---|---|
| 1 | rbcSP/e1/pin 3′//Act1 | rbcSP leaf-specific |
| | P/bar/nos 3′ | promoter driving |
| | | cellulase cDNA of A. cellulolyticus |
| 2 | rbcSP/cbh1/pin 3′//Act1 | rbcSP leaf-specific |
| | P/bar/nos 3′ | promoter driving |
| | | cellulase cDNA of T. reesi |
| 3 | rbcSP/rbcS SP/e1/pin 3′//Act1 | The rbcS SP targets |
| | P/bar/nos 3′ | cellulase of A. cellulolyticus |
| | | into |
| | | maize chloroplasts |
| 4 | rbcSP/rbcS SP/cbh1/pin 3′// | The rbcS SP targets |
| | Act1 P/bar/nos 3′ | cellulase of T. reesi |
| | | into maize chloroplasts |
| Abbreviations: | | |
| The term “rbcSP” means the rice rubisco rbcS promoter region. The rbcSP is a leaf-specific promoter that limits transcription of rbcS to the leaves (Schaeffer and Sheen, Plant Cell 3: 997-1012 (1991)). The nucleotide sequence for the rbcS promoter region is set forth in SEQ ID NO: 1. | | |
| The term “e1” means the cDNA isolated from Acidothermus cellulolyticus which encodes the cellulase EI beta-1,4-endoglucanase precursor. The nucleotide sequence for the gene encoding e1 is set forth in SEQ ID NO: 4. In this example, the codons for the 41 amino acid leader sequence (nucleotides 824 to 946 of SEQ ID NO: 4) are removed. | | |
| The term “cbh1” means the cDNA isolated from Trichoderma reesi that encodes the cellulase cellobiohydrolase. The nucleotide sequence for the gene encoding cbh1 is set forth in SEQ ID NO: 10. In this example, the codons for the 54 amino acid leader sequence (nucleotides 210 to 671 of SEQ ID NO: 10) are removed. | | |
| The term “pin3′” means the potato protease inhibitor II-chloramphenicol acetyltransferase gene's 3′ untranslated sequence which contains transcription termination signals (Thornburg et al., Proc. Natl. Acad. Sci. USA 84: 744-748 (1987)). The pin3′ untranslated sequence includes nucleotides 882 to 1241 of the nucleotide sequence set forth in SEQ ID NO: 15. | | |
| The term “bar” means the phosphinothricin acetyl transferase gene (Thompson et al., EMBO J. 6: 2519-2523 (1987)). The bar gene is a selectable marker for herbicide resistance. The 5′ end of bar is operably linked to the rice actin 1 gene promoter which has been shown to operable in maize (Zhong et al., Plant Physiology 110: 1097-1107 (1996); Zhang et al., Theor. Appl. Genet. 92: 752-761 (1996); Zhang et al., Plant Science 116: 73-84 (1996)). The 3′ end | | |
| # of bar is operably linked to the nos 3′ untranslated sequences. The nucleotide sequence of the bar gene is set forth in SEQ ID NO: 18 (GenBank Accession No. X05822), which encodes the bar having the amino acid sequence from nucleotides 160 to 711. | | |

The term “Act1 P” means the rice Act1 gene promoter which further includes the 5′ intron region (McElroy et al., Mol. Gen. Genet. 231: 150-160 (1991). The sequence of the Act1 gene and its promoter is set forth in SEQ ID NO: 19 (GenBank Accession No. X63830).
The term “nos3′” means the 3′ untranslated sequence from the Agrobacterium nopaline synthase gene encoding nopaline synthase of the amino acid sequence as set forth in SEQ ID NO: 17 which includes nucleotides 2002 to 2521 of SEQ ID NO: 16 (GenBank Accession No. VOOO87J01541). The Nos3′ sequence contains transcription termination signals.
The term “rbcS SP” means the rice rubisco small subunit signal peptide which consists of 47 codons encoding the peptide with the amino acid sequence set forth in SEQ ID NO: 2. The rbcS SP directs the translocation of the rbcS small subunit or any polypeptide to which it is attached to the chloroplasts (Loza-Tavera et al., Plant Physiol. 93: 541-548 (1990)).

Construct 1 contains the rice rubisco rbcS leaf-specific promoter which limits expression of the cellulase encoded by e1 to the cells of the leaves of the maize plant.

Construct 2 contains the rice rubisco rbcS leaf-specific promoter which limits expression of the cellulase encoded by cbh1 to the cells of the leaves of the maize plant.

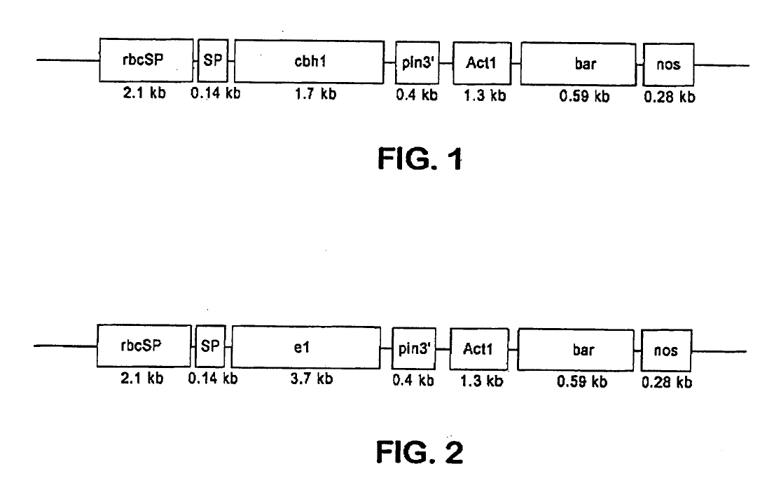

Construct 3, which is shown in FIG. 1, is like construct 1 except that DNA encoding the rbcS SP signal peptide is operably linked to the 5′ end of the e1, and construct 4, which is shown in FIG. 2, is like construct 2 except that DNA encoding the rbcS SP signal peptide is operably linked to the 5′ end of cbh1. Therefore, expression of cellulase from construct 3 or 4, which is limited to the cells of the leaves, directed to the chloroplasts in the cells. All of the above constructs are adjacent to a heterologous gene expression cassette containing the bar gene operably linked to the Act1 promoter.

Construction of plasmid rbcSP/rbcS SP/cbh1//pin3′//Act1P/bar/nos3′. The starting plasmid was pBR10-11 which contained the cry1A(b) gene upstream of the pin31. Between the cry1A(b) and the pin3′ is a DNA polylinker containing in the following order a SmaI, BamHI, SpeI, XbaI, NotI, and EagI restriction enzyme recognition site. The plasmid pBR10-11 (available from Silan Dai and Ray Wu, Department of Molecular Biology and Genetics, Biotechnology Building, Cornell University, Ithaca, N.Y. 14853-2703) was digested with restriction enzymes SpeI and XbaI to produce a 9.2 kb DNA fragment. The 9.2 kb DNA fragment (pBR10-11/SpeI/XbaI/9.2 kb fragment) was purified by agarose gel electrophoresis.

The plasmid pB210-5a (available from William S. Adney, Mike Himmel, and Steve Thomas, National Renewable Energy Laboratory, 1670 Cole Boulevard, Golden Colo. 80401) containing the cbh1 gene from Trichoderma reesei (Trichoderma longibrachiatum)was digested with SpeI and XbaI. The digested plasmid was electrophoresed on an agarose gel and a 1.8 kb fragment (pB210-5a/SpeI/XbaI/1.8 kb fragment containing cbh1) was purified from the gel.

The above 9.2 kb and the 1.8 kb DNA fragments were ligated together using T4 DNA ligase to make plasmid “pBR10-11-cbh1” which was used to transform E. coli XL1 Blue. Transformed bacteria containing plasmid pBR10-11-cbh1 were identified by plating on LB agar gels containing ampicillin.

The plasmid pBR10-11-cbh1 was digested with SmaI and PstI. The PstI end was made blunt with mung bean exonuclease. The digested plasmid was electrophoresed on an agarose gel and the 2.8 kb DNA fragment containing cbh1 and pin3′ was purified from the gel. The purified DNA fragment was designated “cbh1-pin3′/blunt-ended.”

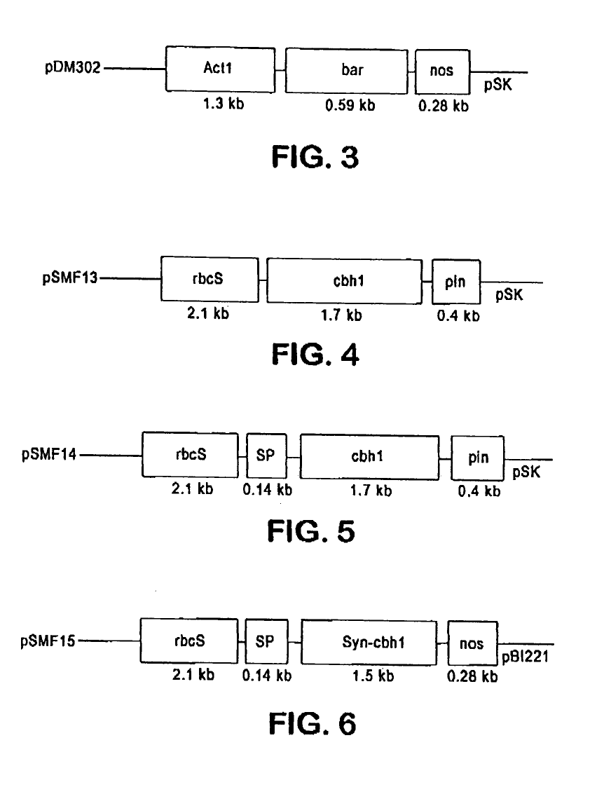

The plasmid pDM302 (Cao et al., Plant Cell Reports 11: 586-591 (1992)), shown in FIG. 3, containing upstream of a ClaI site, a gene cassette consisting of the bar gene flanked by an upstream Act1 promoter and a downstream nos3′, was digested with ClaI. The ClaI ends of the digested plasmid were made blunt with Taq DNA polymerase and the digested plasmid electrophoresed on an agarose gel. The digested plasmid was designated “pDM302/ClaI/blunt-ended.”

The pDM302/ClaI/blunt-ended plasmid and the cbh1-pin3′/blunt-ended DNA fragment were ligated together using T4 DNA ligase to make plasmid “pDM302-cbh1-pin3′” which was used to transform E. coli XL1Blue. Transformed bacteria containing plasmid pDM302-cbh1-pin3′ were identified by plating on LB agar gels containing ampicillin.

Plasmid pDM302-cbh1-pin3′ was digested with SpeI, the ends made blunt with Taq DNA polymerase, and purified by agarose gel electrophoresis. The purified DNA fragment was designated “pDM302-cbh1-pin3′/SpeI/blunt-ended.”

Plasmid pRRI (available from Silan Dai and Ray Wu, Department of Molecular Biology and Genetics, Biotechnology Building, Cornell University, Ithaca, N.Y. 14853-2703), which contains the rice rbcS small subunit gene, was digested with PstI. The rbcS promoter is flanked by PstI sites. The PstI ends were made blunt with mung bean nuclease and the 2 kb DNA fragment (rice rbcS/PstI/blunt-ended) containing the promoter was purified by agarose gel electrophoresis.

Rice rbcSP/PstI/blunt-ended and plasmid pDM-cbh1-pin3′/SpeI/blunt-ended were ligated using T4 DNA ligase to make rbcSP/cbh1/pin3′//Act1P/bar/nos31 which was then used to transform E. coli XL Blue. Transformed bacteria containing plasmid rbcSP/cbh1/pin3′//Act1P/bar/nos3′ were identified by plating on LB agar gels containing ampicillin.

PCR was used to insert NotI sites into rbcSP/cbh1/pin3′//Act1P/bar/nos3′. These sites were used to insert the rice rubisco signal peptide in place of the cbh1 signal peptide. The pRRI plasmid was the source of the rice rubisco signal peptide. It was also the used as a PCR template to produce the PCR product containing the rice rubisco signal peptide flanked by NotI cohesive termini. The rice rubisco signal peptide and the rbcSP/cbh1/pin3′//Act1P/bar/nos3′ plasmid were ligated together using T4 DNA ligase to make rbcSP/rbcS SP/cbh1/pin3′//Act1P/bar/nos3′ which was then used to transform E. coli XL Blue. Transformed bacteria containing plasmid rbcSP/rbcS SP/cbh1/pin3′//Act1P/bar/nos3′ were identified by plating on LB agar gels containing ampicillin.

Construction of plasmid rbcSP/rbcS SP/e1/pin3′//Act1P/bar/nos3′. Plasmid pMPT4-5 (available from William S. Adney, Mike Himmel, and Steve Thomas, national Renewable Energy laboratory, 1670 Colorado Boulevard, Golden, Colo. 80401) contains the e1 gene encoding endoglucanase I from Acidothermus cellulolyticus as a 3.7 kb PvuI DNA fragment cloned into pGEM7 (Promega Corporation, Madison, Wis.). PCR was used to produce a DNA fragment containing the e1 gene flanked by AscI recognition sites. Plasmid rbcSP/cbh1/pin3′//Act1P/bar/nos3′ was also mutagenized by PCR to introduce AscI sites flanking the cbh1 gene. Next, the plasmid rbcSP/cbh1/pin3′//Act1P/bar/nos3′ was digested with AscI and the plasmid free of the cbh1 gene was purified by agarose gel electrophoresis. The AscI flanked e1 gene was ligated using T4 DNA ligase into the rbcSP/cbh1/pin3′//Act1P/bar/nos3′ free of the cbh1 gene to produce plasmid rbcSP/e1/pin3′//Act1P/bar/nos3′, which then used to transform E. coli XL Blue. Transformed bacteria containing plasmid rbcSP/e1/pin3′//Act1P/bar/nos3′ were identified by plating on LB agar gels containing ampicillin.

PCR was used to insert NotI sites into rbcSP/e1/pin3′//Act1P/bar/nos3′. These sites were used to insert the rice rubisco signal peptide in place of the cbh1 signal peptide. The pRRI plasmid was the source of the rice rubisco signal peptide. It was also the used as a PCR template to produce the PCR product containing the rice rubisco signal peptide flanked by NotI cohesive termini. The rice rubisco signal peptide and the rbcSP/e1/pin3′//Act1P/bar/nos3′ plasmid were ligated together using T4 DNA ligase to make rbcSP/rbcS SP/e1/pin3′//Act1P/bar/nos3′ which was then used to transform E. coli XL Blue. Transformed bacteria containing plasmid rbcSP/rbcS SP/e1/pin3′//Act1P/bar/nos3′ were identified by plating on LB agar gels containing ampicillin.

Both heterologous gene expression cassettes are contiguous and the contiguous cassettes can be flanked by MAR sequences.

### EXAMPLE 2

This example shows the construction of plasmids comprising a heterologous gene expression cassette comprising a DNA encoding a cellulase fusion protein. The plasmid constructs are shown in Table 2.

TABLE 2

| | Construct | Plasmid features |
|---|---|---|
| 1 | rbcSP/cbh1/pin 3′ | rbcSP leaf-specific |
| | | promoter driving |
| | | cellulase cDNA of T. reesei |
| 2 | rbcSP/rbcS SP/cbh1/pin 3′ | The rbcS SP targets |
| | | cellulase of T. reesi |
| | | into maize chloroplasts |
| 3 | rbcSP/rbcS SP/syn-cbh1/pin 3′ | The rbcS SP targets |
| | | modified cellulase of T. reesei |
| | | into maize |
| | | chloroplasts |
| 4 | CaMv35s/SSU/e1/nos3′ | The SSU targets the |
| | | cellulase of A. cellulolyticus |
| | | into |
| | | maize chloroplasts |
| 5 | CaMv35s/VSP/e1/nos3′ | The VSP targets the |
| | | cellulase of A. cellulolyticus |
| | | into |
| | | maize apoplasts |
| 6 | CaMv35s/e1/nos3′ | No signal peptide |

Abbreviations:
The term “syn-cbh1” refers to a cbh1 gene that has been codon-modified for use in transformation of tobacco plants. It is available from.
The term “CaMV35s” refers to the cauliflower mosaic virus promoter.
The term “SSU” refers to the glycine max rbcS signal peptide. Glycine max is a soybean and not a rice variety.
The term “VSP” refers to the soybean vegetative storage protein beta signal peptide.

The remainder of the terms in Table 2 are the same as those for table 1.

Construct 1, which is shown in FIG. 4, is plasmid pSMF13 which is plasmid pSK (Stratagene, La Jolla, Calif.) which contains cbh1 operably linked to the rice rubisco rbcS leaf-specific promoter which limits expression of the cellulase encoded by cbh1 to the cells of the leaves of the maize plant.

Construct 2, which is shown in FIG. 5, is plasmid pSF15 which is plasmid pSK which contains cbh1 operably linked to the rice rubisco rbcS leaf-specific promoter which limits expression of the cellulase encoded by cbh1 to the cells of the leaves of the maize plant and a DNA encoding the rbcS SP which targets the cellulase to the chloroplasts.

Construct 3, which is shown in FIG. 6, is like construct 2 except that the cbh1 has been modified to decrease the GC content of the cbh1 to an amount similar to the GC content of the tobacco plant genome. The nucleotide sequence of the modified cbh1 (syn-cbh1) in plasmid pBI221 is set forth in SEQ ID NO:20.

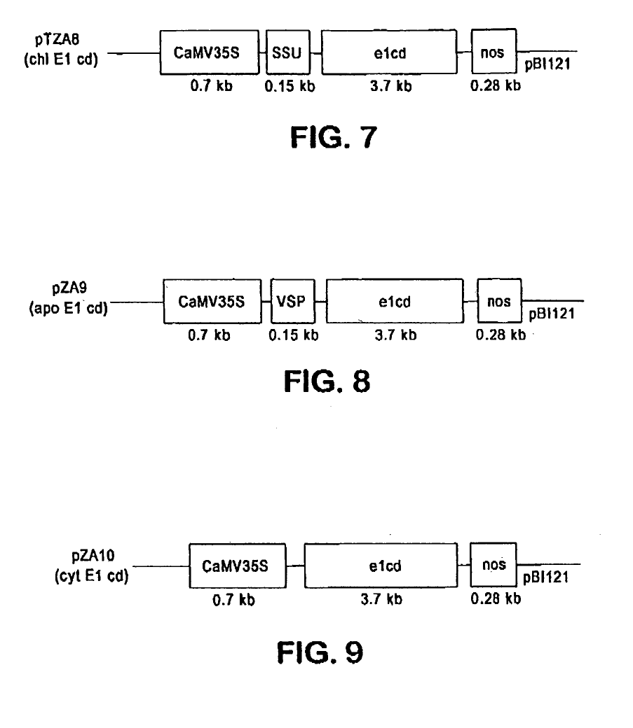

Construct 4, which is shown in FIG. 7, is plasmid pTZA8 which is plasmid pBI121 which contains the caMV35s promoter, which is a constitutive promoter that is active in most plant tissues, to drive expression of e1 which is operably linked to a DNA encoding the SSU signal peptide which targets the cellulase to the chloroplasts.

Construct 5, which is shown in FIG. 8, is plasmid pZA9 which is similar to construct 4 except the signal peptide is encoded by DNA encoding the VSP signal peptide which targets the cellulase to the apoplasts. Construct 6, which is shown in FIG. 9, is plasmid pZA10 which is similar to construct 4 or 5 except that e1 is not operably linked to a DNA encoding a signal peptide that targets the cellulase to a plant organelle.

The constructs were prepared as follows.

First, the plasmid pRR1, which contains the rice rbcS gene was obtained from Ray Wu and Silan Dai, Cornell University. The rice rubisco (rbcS) small subunit was cleaved from pRR1 using EcoRI and EcoRV restriction sites to release a 2.1 kb DNA fragment containing the rbcS. The 2.1 kb DNA fragment was ligated into the plasmid pSK between the EcoRI and EcoRV sites to produce plasmid pSMF8. The 2.1 kb DNA fragment provided the promoter for the cbh1 constructs below.

Next, the cbh1 gene was cloned downstream of rbcS promoter in plasmid pSMF8. First, a 1.7 kb DNA fragment containing the cbh1 gene from Trichoderma reesei was isolated from plasmid pB210-5A (available from William S. Adney, Mike Himmel, and Steve Thomas, National Renewable Energy Laboratory; described in Shoemaker et al., Bio/Technology 1: 691-696 (1983)) by digesting with SalI and XhoI. The ends of the 1.7 kb DNA fragment were made blunt end using DNA polymerase I (large fragment). The blunt-ended DNA fragment was cloned into plasmid pSMF8, which had been digested with BamHI and the ends made blunt with DNA polymerase I, to make plasmid pSMF9.

Next, to complete the heterologous gene expression cassette, the pin3′ transcription termination nucleotide sequence was inserted at the 3′ end of the cbh1 gene in plasmid pSMF9. The pin3′ transcription termination nucleotide sequence was cleaved from pBR10-11 with PstI. However, to remove the pin3′ transcription termination nucleotide sequence from pBR10-11, a PstI site had to be introduced upstream of the pin31 transcription termination nucleotide sequence.

To generate the PstI site upstream of the pin3′ transcription termination nucleotide sequence in pBR10-11, the pBR10-11 was digested with NotI and XhoI and a 70 bp multi-cloning site nucleotide sequence, which had been isolated from the pSK vector by digesting with NotI and XhoI, was cloned between the NotI and XhoI sites of the pBR10-11 to produce plasmid pSMF11. The pin3′ transcription termination nucleotide sequence was then removed from pSMF11 by digesting with PstI to produce a 1 kb DNA fragment which was then cloned into the PstI site of pSK, which had been digested with PstI, to produce plasmid pSMF12. PSMF12 was then digested with NotI to produce a 1 kb DNA fragment containing the pin3′ transcription termination nucleotide sequence. The 1 kb DNA fragment cloned into the NotI site downstream of the cbh1 gene in pSMF9, which had been digested with NotI, to produce plasmid pSMF13 (construct 1 in Table 2).

Next, a DNA encoding a signal peptide which targets proteins to which it is attached to the chloroplasts was inserted upstream of the cbh1 and in the same reading frame as the cbh1. Thus, a fusion protein is produced from translation of RNA transcribed from the cbh1 DNA linked to the DNA encoding the signal peptide. DNA encoding the signal peptide (SP) was isolated from the rbcS in the pRRI plasmid. Because there were no convenient restriction enzyme sites available which flanked the DNA encoding the SP for cloning, it was planned to PCR amplify that region containing the DNA encoding the SP using PCR primers with PCR primers that contained convenient restriction enzyme sites for cloning. At the end of the rbcS promoter pSMF13 is a unique AvrII site and upstream of the first ATG of the cbh1 gene is a unique BsrGI. A DNA encoding the SP that was flanked with an AvrII site on one end and a BsrGI site on the opposite end would be able to be cloned between the AvrII and BsrGI sites in PSMF13. That would place the DNA encoding the SP between the rbcS promoter and the cbh1 gene and would enable a fusion protein containing the SP fused to the cellulase.

Therefore, PCR primers were synthesized using DNA sequences for the AvrII and BsrGI sites and the SP DNA sequences. The upstream PCR primer (SPLF) had the nucleotide sequence 5′-CCGCCTAGGCGCATGGCCCCCTCCGT-3′ (SEQ ID NO:21) and the downstream PCR primer (SP3R) had the nucleotide sequence 5′-CGCTGTACACGCACCTGATCCTGCC-3′ (SEQ ID NO:22). Plasmid pRR1 encoding the SP was PCR amplified with the PCR primers and the 145 bp amplified product was purified using 2% agarose gel. The purified 145 bp product was sequenced to confirm that the 145 bp amplified product contained the SP nucleotide sequences. The amplified product was digested with AvrII and BsrGI and cloned between the AvrII and BsrGI sites of pSMF13 digested with AvrII and BsrGI to produce plasmid pSMF14.

To produce pSMF15 which contains a cbh1 gene codon-modified to decrease the GC content of the cbh1 gene to an amount similar to the GC content of the tobacco genome, a synthetic cbh1 (syn-cbh1) gene was obtained from plasmid pZD408 (available from Ziyu Dai, Pacific Northwest national Laboratory, 902 Battelle Boulvard, Richland, Wash. 99352). The syn-cbh1 is a cbh1 which had been codon-modified for use in tobacco plant transformations. The nucleotide sequence of syn-cbh1 is set forth in SEQ ID NO:20. Plasmid pZD408 was linearized with NcoI and the ends made blunt. Then, the blunt-ended pZD408 was digested with HindIII to remove the CaMV35S promoter. A 4.5 kb DNA fragment containing the syn-cbh1 was isolated from the CaMV35S promoter by agarose gel electrophoresis. The 4.5 kb DNA fragment was dephosphorylated and the DNA fragment containing a blunt end and a HindIII end was named pZD408B.

Plasmid pSMF14 was digested with BsrGI, the BsrGI ends made blunt, and then pSMF14 was digested with HindIII to produce a DNA fragment containing the rbcS promoter with the DNA encoding the SP flanked by a blunt end and a HindIII end. The DNA fragment was purified by agarose gel electrophoresis and ligated to the pZD408B DNA fragment to produce plasmid pSMF15 (construct 3 of Table 2).

The Heterologous Gene Expression Cassettes are contiguous can be flanked by MAR sequences.

### EXAMPLE 3

This example shows the construction of plasmids comprising a heterologous gene expression cassette comprising a DNA encoding a ligninase fusion protein and a heterologous gene expression cassette comprising a DNA encoding the bar gene. The constructs are shown in Table 3.

TABLE 3

| | Construct | Plasmid features |
|---|---|---|
| 1 | rbcSP/ckg4/pin 3′//Act1 | rbcSP leaf-specific |
| | P/bar/nos 3′ | promoter driving ckg4 |
| | | cDNA of P. chrysosporium |
| 2 | rbcSP/ckg5/pin 3′//Act1 | rbcSP leaf-specific |
| | P/bar/nos 3′ | promoter driving ckg5 |
| | | cDNA of P. chrysosporium |
| 3 | rbcSP/rbcS SP/ckg4/pin | The rbcS SP targets ckg4 |
| | 3′//Act1 P/bar/nos 3′ | into maize chloroplasts |
| 4 | rbcSP/rbcS SP/ckg5/pin 3′// | The rbcS SP targets ckg5 |
| | Act1 P/bar/nos 3′ | into maize chloroplasts |

Abbreviations:
The terms “ckg4” and “ckg5” mean the ligninase cDNAs isolated from the basidiomycete Phanerochaete. chrysosporium, SEQ ID NO: 11 and SEQ ID NO: 13, respectively. The codons for the 28 amino acid leader are deleted so that the expressed gene product remains inside the cells.

The remainder of the terms in Table 3 are the same as those for Table 1. All plasmid constructs contain the selectable marker gene (bar) driven by the rice actin 1 gene promoter. The rice actin gene and its promoter are disclosed in U.S. Pat. No. 5,641,876 to McElroy et al.

Construct 1 contains the rice rubisco rbcS leaf-specific promoter which limits expression of the ligninase encoded by ckg4 to the cells of the leaves of the maize plant.

Construct 2 contains the rice rubisco rbcS leaf-specific promoter which limits expression of the ligninase encoded by ckg5 to the cells of the leaves of the maize plant.

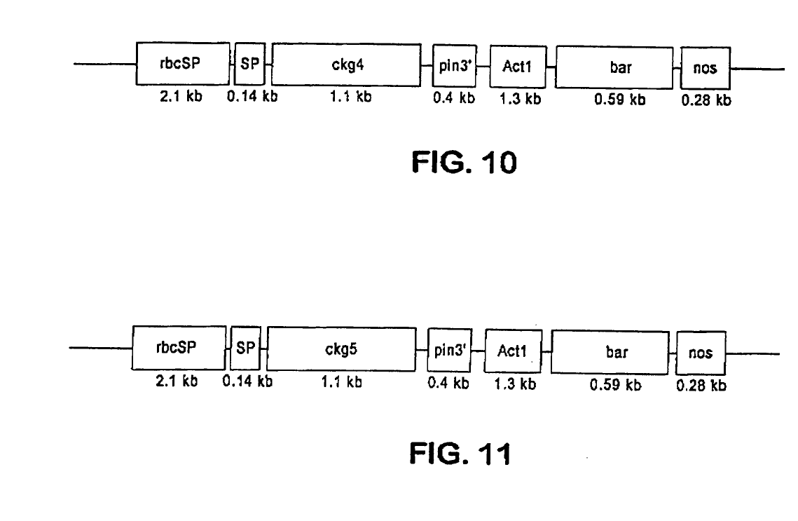

Construct 3, which is shown in FIG. 10, contains the rice rubisco rbcS leaf-specific promoter which limits expression of the ligninase encoded by ckg4 to the cells of the leaves of the maize plant and further contains DNA encoding the rbcS SP which targets the ligninase to the chloroplasts.

Construct 4, which is shown in FIG. 11, contains the rice rubisco rbcS leaf-specific promoter which limits expression of the ligninase encoded by ckg5 to the cells of the leaves of the maize plant and further contains DNA encoding the rbcS SP which targets the ligninase to the chloroplasts. All of the above constructs are adjacent to a heterologous gene expression cassette containing the bar gene operably linked to the Act1 promoter. Both heterologous gene expression cassettes are contiguous and the contiguous cassettes can be flanked by MAR sequences.

### EXAMPLE 4

This example shows the construction of plasmids comprising a heterologous gene expression cassette comprising a DNA encoding a ligninase fusion protein. The constructs are shown in Table 4.

TABLE 4

| | Construct | Plasmid features |
|---|---|---|
| 1 | rbcSP/ckg4/pin 3′ | rbcSP leaf-specific |
| | | promoter driving ckg4 cDNA |
| | | of P. chrysosporium |
| 2 | rbcSP/ckg5/pin 3′ | rbcSP leaf-specific |
| | | promoter driving ckg5 cDNA |
| | | of P. chrysosporium |
| 3 | rbcSP/rbcS SP/ckg4/pin 3′ | The rbcS SP targets ckg4 |
| | | into maize chloroplasts |
| 4 | rbcSP/rbcS SP/ckg5/pin 3′ | The rbcS SP targets ckg5 |
| | | into maize chloroplasts |

The terms in table 4 are the same as those for Tables 1 and 3.

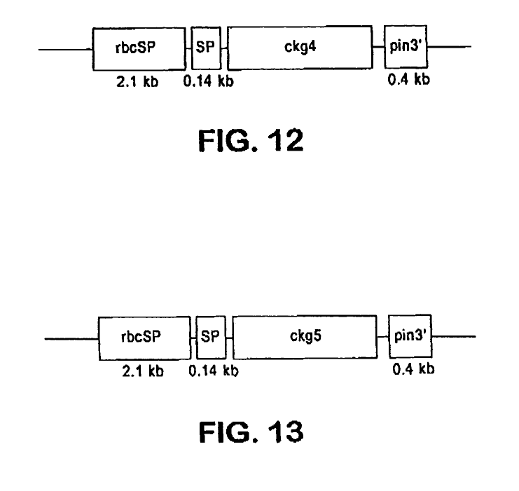

Construct 1, which is shown in FIG. 12, is plasmid pSMF18 which is plasmid pSK which contains the rice rubisco rbcS leaf-specific promoter which limits expression of the ligninase encoded by ckg4 to the cells of the leaves of the maize plant.

Construct 2, which is shown in FIG. 13, is plasmid pSMF19 which is plasmid pSK which contains the rice rubisco rbcS leaf-specific promoter which limits expression of the ligninase encoded by ckg5 to the cells of the leaves of the maize plant.

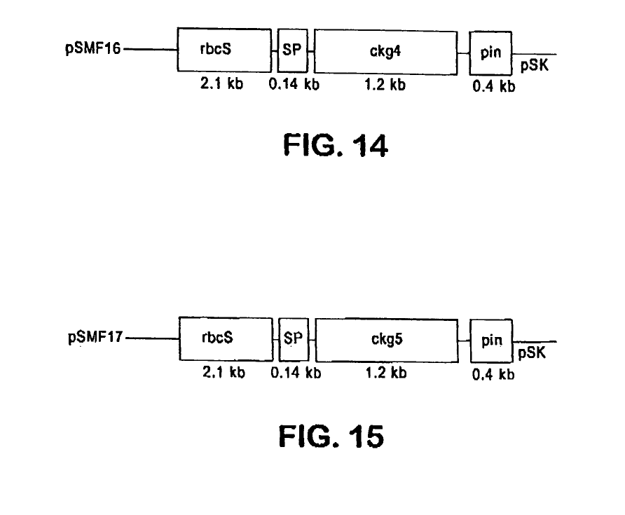

Construct 3, which is shown in FIG. 14, is plasmid pMSF16 which is plasmid pSK which contains the rice rubisco rbcS leaf-specific promoter which limits expression of the ligninase encoded by ckg4 to the cells of the leaves of the maize plant and further contains DNA encoding the rbcS SP which targets the ligninase to the chloroplasts.

Construct 4, which is shown in FIG. 15, is plasmid pSMF17 which is plasmid pSK which contains the rice rubisco rbcS leaf-specific promoter which limits expression of the ligninase encoded by ckg5 to the cells of the leaves of the maize plant and further contains DNA encoding the rbcS SP which targets the ligninase to the chloroplasts. The above heterologous gene expression cassettes can be flanked by MAR sequences.

The ligninase constructs shown in Table 4 are prepared as described below.

Two plasmids, pCLG4 and pCLG5, the former containing a cDNA clone encoding the ligninase gene ckg4 and the latter containing a cDNA clone encoding the ckg5 were obtained from Dr. C. Adinarayana Reddy, Department of Microbiology and Public Health, Michigan State University and described in de Boer et al., Gene 60: 93-102 (1987), Corrigendum in Gene 69: 369 (1988). These ligninase cDNA clones were prepared from a white-rot filamentous fungus (Phanerochaete chrysosporium). The cDNAs for ckg4 and ckg5 had each been cloned into the PstI site of the pUC9 plasmid to make pCLG4 and pCLG5, respectively. The codons for the 28-amino acid leader sequence is deleted from both cDNAs before cloning so that expressed gene product remains inside the cell.

Plasmid pSMF16 is made as follows. The ckg4 gene is removed from pCLG4 by digesting the plasmid with the restriction enzymes XbaI and BstEII to produce a 1.2 kb DNA fragment containing the ckg4 without the nucleotide sequence encoding the transit peptide. The BstEII removes the nucleotide sequences encoding the transit peptide of the ligninase.

The ends of the DNA fragment containing the ckg4 gene are made blunt and the blunt-ended DNA fragment is ligated into pSMF14 in which the cbh1 has been removed by digesting with BsrGI and XhoI and the ends made blunt to produce pSMF16.

Plasmid pSMF18 is made as follows. The nucleotide sequence encoding the rbcS signal peptide and cbh1 are removed from pSMF14 by digesting pSMF14 with AvrII and XhoI instead of BsrGI and XhoI. The ends of the digested pSMF14 are made blunt and the blunt-ended DNA fragment containing the ckg4 gene, prepared as above, is ligated into the digested pSMF14 to make plasmid pSMF18.

Plasmid pSMF17 is made as follows. The ckg5 gene is removed from pCLG5 by digesting the plasmid with the restriction enzymes XbaI and EagI to produce a 1.2 kb DNA fragment containing the ckg5 without the nucleotide sequence encoding the transit peptide. The EagI removes the nucleotide sequences encoding the transit peptide of the ligninase.

The ends of the DNA fragment containing the ckg5 are made blunt and the blunt-ended DNA fragment is ligated into pSMF14 in which the cbh1 has been removed by digesting with BsrGI and XhoI and the ends made blunt to produce pSMF17.

Plasmid pSMF19 is made as follows. The nucleotide sequence encoding the rbcS signal peptide and cbh1 are removed from pSMF14 by digesting pSMF14 with AvrII and XhoI instead of BsrGI and XhoI. The ends of the digested pSMF14 are made blunt and the blunt-ended DNA fragment containing the ckg5 gene, prepared as above, is ligated into the digested pSMF14 to make plasmid pSMF19.

### EXAMPLE 5

This example shows the transformation of maize multi-meristem primordia via Biolistic bombardment with the plasmid constructs of Examples 1-4, regeneration of the transgenic plants, confirmation of the integration of the plasmid constructs into the plant genome, and confirmation of the expression of the cellulase or ligninase fusion proteins in the transgenic plant. For transformations with the constructs of Examples 2 and 4, which do not contain a selectable marker, a selectable marker comprising the bar gene in the plasmid pDM302 (Cao et al., Plant Cell Reports 11: 586-591 (1992)) is cotransfected into the cells with the plasmid containing the ligninase or cellulase heterologous gene expression cassette.

Maize seeds have been germinated in Murashige and Skoog (MS) medium (Murashige and Skoog, Physiol. Plant 15: 473-497 (1962)) supplemented with the appropriate growth regulators (Zhong et al., Planta 187: 490-497 (1992)). Shoot meristems have been dissected and cultured for 2-3 weeks until an initial multiplication of meristem have been produced for bombardment.

The multi-meristem primordia explants are bombarded with tungsten particles coated with particular plasmids of Example 1 or 3 or with particular plasmids of Example 2 or 4 along with the plasmid containing the heterogenous gene expression cassette containing the bar gene. The bombarded explants are gently transferred onto meristem multiplication medium for further multiplication, about 6 to 8 more weeks. This step is required to reduce the degree of chimerism in transformed shoots prior to their chemical selection. Shoots are transferred to the above medium containing 5 to 10 mg per liter glufosinate ammonium (PPT) selectable chemical for another 6 to 8 weeks. Chemically selected shoots are rooted in rooting medium containing the same concentration of PPT. Plantlets are transferred to pots, acclimated, and then transferred to a greenhouse.

When the plantlets or shoots are small, the quantity of transgenic plant material is insufficient for providing enough DNA for Southern blot hybridization; therefore, polymerase chain reaction (PCR) is used to confirm the presence of the plasmid constructs the plantlets. The amplified DNA produced by PCR is resolved by agarose or acrylamide gel electrophoresis, transferred to membranes according standard Southern transfer methods, and probed with the appropriate DNA construct or portion thereof according to standard Southern hybridization methods. Those shoots or plantlets which show they contain the construct in its proper form are considered to have been transformed. The transformed shoots or plantlets are grown in the greenhouse to produce sufficient plant material to confirm that the plasmid constructs has been properly integrated into the plant genome. To confirm proper integration of the plasmid constructs into the plant genome, genomic DNA is isolated from the greenhouse grown transgenic plants and untransformed controls and analyzed by standard Southern blotting methods as in Zhong et al., Plant Physiology 110: 1097-1107 (1996); Zhang et al., Theor. Appl. Genet. 92: 752-761 (1996); Zhang et al., Plant Science 116: 73-84 (1996); and, Jenes et al., In Transgenic Plants. Vol. 1. Kung, S-D and Wu, R (eds.). Academic Press, San Diego, Calif. pp. 125-146 (1992).

To confirm expression of the ligninase or cellulase fusion protein, total cellular RNA is isolated from the greenhouse grown plant tissues as described in Zhong et al., Plant Physiology 110: 1097-1107 (1996). The mRNA encoding the cellulase or ligninase fusion protein is detected by RNA Northern blot analysis using the same probes used for the Southern blot analyses. Briefly, the RNA is electrophoresed on a denaturing formaldehyde agarose gel, transferred to nitrocellulose or nylon membranes, hybridized to the appropriate ligninase or cellulase probe, and then exposed to X-ray autoradiology film. The hybridization bands are scanned using a densitometer which enables determination of the expression level of the specific mRNA.

Translation of the mRNA is confirmed by Western blot analysis according to the standard methods of Towbin et al., Proc. Natl. Acad. Sci. USA 76: 4350 (1979) and Burnette, Anal. Biochem. 112: 195 (1981) using antibodies specific for ligninase or cellulase.

### EXAMPLE 6

Transgenic maize containing both a ligninase and a cellulase fusion protein is made by crossing-breeding the abovementioned transgenic plants one of which contains cbh1 or e1 stably integrated into the genome and the other of which contains ckg4 or ckg5 stably integrated into the genome using the method provided in (Zhang et al, Theor. Appl. Genet. 92: 752-761, (1996); Zhong et al, Plant Physiol. 110: 1097-1107, (1996); Zhong et al, Planta, 187: 483-489, (1992)). Transgenic plants that carry a low copy number of the DNA encoding the ligninase or cellulase fusion proteins are used for cross-breeding.

Briefly, transgenic maize plants that produce the ligninase fusion protein are made as disclosed in Example 5 to make a first transgenic plant and transgenic maize plants that produce the cellulase fusion protein are made as disclosed in Example 5 to make a second transgenic plant. The first and second transgenic plants are cross-pollinated to create a transgenic plant which produces both a ligninase and a cellulase fusion protein. The progeny are analyzed for homozygosity and transgenic plants that are homozygous for both the ligninase gene cassette and the cellulase gene cassette are selected for further propagation for seeds.

The progeny in the above crosses are used in subsequent crosses to produce transgenic maize with both ligninase gene cassettes and one, two, or three cellulase gene cassettes or transgenic maize with two or three cellulase gene cassettes and one ligninase gene cassette.

### EXAMPLE 7

Production levels and activity of the cellulase fusion protein in transgenic maize made as in Example 5 or 6 is determined as follows.

Cellulase activity in transgenic maize is first assayed by standard methods (Ghose. In Analytical Method B-304, rev. A, IUPAC Commission on Biotechnology. A short Report (1984)) based on the time course assay for hydrolysis of a pre-weighed sample of filter paper at pH 4.8-5.2 and temperature of 50° C. While the filter paper assay is a standard substrate for cellulase activity, results using the filter paper assay are not particularly representative of the actual activity of the cellulase in plant materials containing cellulose, hemicellulose, and other sugars or sugar polymers. Therefore, a more accurate method for determining cellulase activity is used.

Plant material is ground and the ground material is suspended to a concentration of up to about 5% in 0.05 M citrate buffer at pH 4.8 and incubated with shaking at 50° C. Over a 48 hour time period, samples are removed at intervals of 0, 1, 3, 12, 24, and 48 hours. A minimal amount of sodium azide, about 0.05%, is added to the citrate buffer during incubation to control microbial activity. For analysis by high pressure liquid chromatography (HPLC), the supernatant fraction of each sample is removed, capped, and heated to inactivate the enzymes. The inactivated supernatant fraction is filtered through a syringe filter and analyzed by HPLC to measure the glucose, cellobiose, and xylose content of the samples according to established methods (Dale et al., Biosource Technol. 56: 11-116 (1996)).

Cellulase activity is manifested by an increasing level of glucose, xylose and/or cellobiose levels in the supernatant fractions during the 48 hour period. The control for the above assay is to treat samples from non-transgenic plants with varying amounts of commercially available cellulase enzymes such as CYTOLASE 300 which is a cellulase from Genencor, Inc. and NOVOZYME 188 which is a cellobiose from Novo Laboratories, Inc. to confirm that the ground plant material is susceptible to hydrolysis.

### EXAMPLE 8

Comparison of cellulase activity in transgenic maize prepared as in Example 5 or 6 treated to enhance cellulose accessibility.

Generally, cellulose and hemicellulose in plant material are not very accessible to hydrolytic enzymes such as cellulase. Therefore, it is possible that even if the cellulase fusion protein is produced in the transgenic plants of the present invention, its cellulase activity would not be measurable. Therefore, to demonstrate accessibility, samples of the transgenic maize plants of the present invention are treated by the ammonia fiber explosion process to increase cellulose and hemicellulose accessibility (Dale et al., Biosource technol. 56: 11-116 (1996)). Samples treated are analyzed as in Example 3.

In previous experiments with coastal Bermuda grass, the ammonia fiber explosion process disrupted the plant cell walls sufficiently to permit over 80% extraction of plant protein, compared with less than 30% extraction under the same conditions prior to ammonia treatment (de la Rosa et al., Appl. Biochem. Biotechnol. 45/46: 483-497 (1994). The process increased the hydrolytic effectiveness of the added cellulases by at least six-fold (Dale et al., Biosource Technol. 56: 11-116 (1996)). Thus, it is expected that the ammonia fiber explosion process helps release cellulase from the transgenic maize chloroplasts and will also increase the access of the cellulase released to the cellulose in the plant material.

### EXAMPLE 9

Production levels and activity of the ligninase fusion protein in transgenic maize made as in Example 5 or 6 can be determined as follows.

Maize leaves from the transgenic maize made as in Examples 5 or 6 are ground using a pestle and mortar. Chloroplasts are isolated from leaves of transgenic plants by Ficoll (Pharmacia) gradient centrifugation and ground as above.

The ground materials (leaves, grains, chloroplasts) are suspended in 50 mM L-tartrate buffer (pH 4.5), mixed well by vortexing, and centrifuged for 10 minutes at 14,000 rpm (16,000×g) at 4° C. and the supernatant fraction tested for lignin peroxidase (LIP) activity as described in Tien et al., Meth. Enzymol. 161: 238-249 (1988). The LIP assay measures the production of veratraldehyde (as an increase in absorbance at 310 nm) from veratryl alcohol (substrate) in the presence of hydrogen peroxide. Control assays are done on non-transgenic maize seeds to measure endogenous peroxidase activity. The assay is sensitive and is able to detect very low levels of lignin peroxidase activity, e.g., conversion of 0.1 mmole substrate per minute per liter of test sample.

Soluble protein content is determined by the Bradford procedure (Bradford, Anal. Biochem. 72: 248-254 (1976)) using bovine serum albumen (BSA) as the standard. LIP enzyme in the extracted fluid is purified by Fast Protein liquid Chromatography (FPLC) analysis using the Mono Q anion exchange system (Pharmacia) and a gradient of 0 to 1 M Na-acetate to elute the various isozymes (Yadav et al., Appl. Environ. Microbiol. 61: 2560-2565 (1995); Reddy et al., FEMS Microbiol. Rev. 13: 137-152 (1994)). The relative activity, yield, pH optimum, stability, and other characteristics of the LIP in the transgenic plant are compared to that determined for the LIP isolated from the fungus. Furthermore, ground maize seeds or leaf extracts containing the LIP is used to treat various lignocellulosic feeds in small laboratory reactor systems and the extent of delignification can be analyzed per established procedures (Van Soest et al., Assoc. Off. Anal. Chem. J. 51: 780-785 (1968)).

Detection of ligninase mRNA is by isolating the mRNA from the transgenic plants as above, resolving the mRNA by denaturing RNA gel electrophoresis, transferring the resolved mRNA to membranes, and probing the membranes with ckg4 or ckg5 cDNA probes.

Western blots are performed to determine whether the LIP protein is in an active or inactive form. The total protein from the transgenic plants is resolved by SDS-polyacrylamide gel electrophoresis and transferred to membranes. The membranes are probed with antibodies to LIP H2 (ckg4) or LIP H10 (ckg5).

### EXAMPLE 10

This Example illustrates the delay in flowering and increase in biomass of transgenic tobacco expressing the Arabidopsis floral repressor gene Flowering Locus C.

Flowering Locus C (FLC), a gene from Arabidopsis thaliana (L.) Heynh. that acts as a flowering repressor, was expressed in tobacco (Nicotiana tabacum L. ‘Samsun’). Five putative transgenic lines were selected and examined for the presence of FLC. Genomic DNA and total RNA were isolated from the leaves and used for polymerase chain reaction (PCR) and RNA blot analysis, respectively. Both DNA and RNA tests confirmed the integration and transcription of FLC in all five lines and their T₁ progenies. Transgenic plants in one line showed an average of 36 d delay in flowering time compared to control plants, and the overall mean for all lines was 14 d. Transgenic plants also displayed increased leaf size and biomass yield and reduced height at flowering time. It is important to note that the delay in flowering might have been caused by a slower rate of leaf initiation (i.e. nodes/day) rather than by a change in the flowering mechanism itself.

Flowering, the switch from vegetative to reproductive growth, is a key developmental change in the life cycle of the plant (Simpson and Dean, Science, 296: 285-289 (2002); Henderson and Dean, Development 131: 3829-3838 (2004)) and is controlled by both environmental and developmental signals (Jang et al., J. Plant Biotechnol, 5: 209-214 (2003)). The control of flowering and genes associated with the mechanism have recently been reviewed (Reeves and Coupland, Curr Opin Plant Biol 3: 37-42 (2000); Samach and Coupland, Bioassays, 22: 38-47 (2000); Araki, Curr Opin Plant Biol, 4: 63-68 (2001); Mouradov et al., Plant Cell, 14: 5111-5130 (2002); Simpson and Dean, ibid, 2002; Henderson and Dean, ibid, 2004). Because of its importance, flowering is the subject of intense studies, but it is still poorly understood at the molecular level partly due to the complexity of the flower initiation process (Koornneef et al., Genetics 148: 885-892 (1998)). Regardless, progress is regularly being made in this area. Most molecular and genetic studies on flowering have been carried out on the model plant, Arabidopsis thaliana (L.) Heynh.

The most current flowering model (Henderson and Dean, ibid, 2004) shows the involvement of at least eight distinct pathways regulating the change from vegetative growth to reproductive organ development. Although these pathways act largely independent of one another, some interaction does take place among them (Koornneef et al., ibid, 1998; Rouse et al., Plant J., 29: 183-191 (2002)). Flowering is promoted by the light quality, ambient temperature, gibberellin, circadian clock, and photoperiod pathways (Henderson and Dean, ibid, 2004). Acting antagonistically to these pathways is the floral repressor gene FLOWERING LOCUS C (FLC). Several genes act to promote FLC expression; however, FLC is down-regulated by vernalization (a long period of near-freezing temperatures) and the autonomous pathway genes (Michaels and Amasino, Plant Cell, 11: 949-956 (1999) and Michaels and Amasino, Plant Cell, 13: 935-941 (2001); Sheldon et al., Plant Cell, 11: 445-458 (1999) and Sheldon et al., Proc Natl Acad Sci USA 97: 3753-3758 (2000); Henderson and Dean, ibid, 2004). FLC encodes a MADS box transcription factor that is expressed mainly in vegetative shoot apices and roots (Michaels and Amasino, ibid, 1999). FLC works to inhibit flowering by suppressing a group of floral promotion genes termed ‘floral pathway integrators’ (Michaels and Amasino, ibid, 1999 and Michaels and Amasino, ibid, 2001; Sheldon et al., ibid, 1999; Henderson and Dean, ibid, 2004). Plants over-expressing FLC experience an extended vegetative growth phase unless a vernalization requirement is met (Michaels and Amasino, ibid, 1999).

Control of flowering time is essential for efficient seed production and for summer cultivation of biennial leafy crops (Jang et al., ibid, 2003). Since delay in flowering time results in prolonged vegetative growth, it theoretically may produce higher yields in crops grown for their leaves and/or biomass. Another key application for flowering delay is bioconfinement of transgenic pollen to avoid transfer of transgenes to cross-breedable non-target crops.

To test the delay in flowering and increase in biomass production, we used the model plant tobacco (Nicotiana tabacum L.). In this study, we describe Agrobacterium-mediated transformation of tobacco with constitutively expressed FLC and the molecular and physiological analyses of the transgenic plants.

Materials and Methods

Plant materials: Tobacco (Nicotiana tabacum L. ‘Samsun’) seeds were prewashed in water with 0.2% Tween-20 for 10 min and rinsed three times with distilled water. The seeds were surface sterilized with 70% (v/v) ethanol for 1 min followed by immersion in 20% (v/v) Clorox (5.25% sodium hypochlorite) for 20 min and then rinsed three times with sterilized double-distilled water. Seeds were germinated on Murashige and Skoog, ibid, (1962) (MS) basal medium (Sigma-Aldrich, St. Louis, Mo.) with 30 g/L sucrose, and solidified with 2.5 g/L gelrite (Sigma-Aldrich, St. Louis, Mo.). Cultures were kept under 30 μmol/m²/s continuous white deluxe fluorescent light at 25° C. Leaf segments (0.5×0.5 cm²) were aseptically excised from the second and third fully expanded in vitro produced leaves (Horsch et al., Science, 227: 1229-1231 (1985)) for infection with Agrobacterium tumefaciens.

Agrobacterium strain, plasmid, and in vitro culture: The transformation experiments were conducted using A. tumefaciens strain GV 3101 (pMP90RK) (Koncz and Schell, Mol. Gen. Genet 204: 383-396 (1986)) containing the 3.232 kb binary vector pGreen (Hellens et al., Plant Mol. Biol., 42: 819-832 (2000a)). The plasmid contains FLC from Arabidopsis thaliana and the phosphoinothricin acetyltransferase gene (bar), both under the control of the constitutive cauliflower mosaic virus (CaMV) 35S promoter and the nopaline synthase (nos) terminator.

Agrobacterium containing the transgenes was grown in 10 mL YEP medium (containing 10 g/L Bacto-peptone, 10 g/L Bacto yeast extract, 5 g/L NaCl, pH 7.2) supplemented with 25 mg/L of both kanamycin and gentamycin (Hellens et al., Trends Plant Sci, 5: 446-451 (2000b)), incubated at 28° C. and 250 rpm for 48 h, and the cultures (cell density 0.6-0.8 at A₆₀₀) were used for transformation.

Sensitivity of Tobacco Leaves to glufosinate ammonium. Since optimization of the herbicide concentration is a prerequisite for the selection efficiency of transformed lines, a kill curve was developed to test the sensitivity of tobacco leaf segments to glufosinate ammonium. Glufosinate ammonium was used as a selection agent since the binary vector used in this study contained bar gene. Twenty of the 3-week old tobacco leaf segments were placed on MS medium containing 0, 2.5, 5.0, 7.5, 10.0, 12.5 and 15.0 mg/L of glufosinate ammonium for 2 weeks. The explant survival was recorded. All explants turned brown and died after being cultured on glufosinate ammonium with concentrations of 5 mg/L or more. Therefore, 5 mg/L glufosinate ammonium was used for further transgenic plant selection.

Inoculation and co-cultivation: Leaf segments were infected using the Agrobacterium culture at room temperature for 20-25 min. After inoculation, the leaf explants were blotted on sterilized filter papers and placed abaxial side down on MS medium supplemented with 4.5 μmol/L N⁶-benzylamino purine (BAP) and 0.5 μmol/L α-naphthaleneacetic acid (NAA) (Ziegelhoffer T., Will, J., and Austin-Phillips, S. (1999). Expression of bacterial genes in transgenic alfalfa (Medicago sativa L.), potato (Solanum tuberosum L.) and tobacco (Nicotiana tabacum L.) Mol. Breed. 5: 309-318), 30 g/L sucrose and 2.5 g/L gelrite. They were co-cultivated for 2 d under continuous light as described above for seed culture. Then, they were rinsed three times with sterilized distilled water containing 400 mg/L carbencillin to prevent Agrobacterium overgrowth, blotted onto sterilized filter papers and placed adaxial side down on the same co-cultivation medium supplemented with 400 mg/L carbencillin and 5 mg/L glufosinate ammonium for selection of the putative transformants. The callus containing the adventitious shoots was subcultured in the same medium, and then shoots were excised and rooted on half strength MS medium containing 400 mg/L carbencillin and 5 mg/L glufosinate ammonium in Magenta boxes (Sigma-Aldrich, St. Louis, Mo.). Plantlets were transferred to the greenhouse after acclimatization. Greenhouse conditions were 25-28° C., 90-95% humidity and 190 μmol/m²/s light.

Polymerase chain reaction (PCR) screening: After selection on glufosinate ammonium-containing medium, PCR analysis was used to screen the T₀ plants for FLC transgene incorporation. Five independent putative transgenic lines were selected for PCR analysis. Total genomic DNA of control and T₀ plants were extracted from leaves as described by Edwards et al., Nucl. Acids Res, 19: 1349 (1991). The following set of primers was used: FLC F, 5′-CGA TAA CCT GGT CAA GAT CC-3′ (forward primer, SEQ ID NO:25) and FLC R, 5′-CTG CTC CCA CAT GAT GAT TA-3′ (reverse primer, SEQ ID NO:26). The predicted size of the amplified DNA fragments of the transgene was 338 bp. DNA amplifications were performed in a thermo cycler (Perkin Elmer/Applied Biosystem, Foster City, Calif.) using REDTaq™ ReadyMix™ PCR Reaction Mix with MgCl₂ (Sigma-Aldrich, St. Louis, Mo.). The PCR profile had an initial denaturation step at 94° C. for 1 min, followed by 30 cycles of 1 min at 94° C. (denaturation), 2 min at 60° C. (annealing) and 3 min at 72° C. (extension). The reaction mixture was loaded directly onto a 1.0% (w/v) agarose gel, stained with ethidium bromide and visualized with UV light.

RNA gel blot analysis: Total RNA of control, T₀ and T₁ plants from five putative lines was isolated from leaves of 6-weeks-old greenhouse plants using the TRI Reagent (Sigma-Aldrich, St. Louis, Mo.) according to the manufacturer's instructions. Aliquots of RNA (20 μg) were fractionated in 1.2% agarose formaldehyde denaturing gel and blotted on a Hybond-N⁺ nylon membrane (Amersham Pharmatica Biotech) as specified by the manufacturer. The probe was generated by digesting plasmid DNA with XhoI and SpeI, releasing the 0.59-kb fragment containing the FLC coding region. The digestion reaction mixture was gel-purified using the QIAquick Gel Extraction Kit (QIAGEN Inc., Valencia, Calif.). Probe labeling and transcript detection were obtained using the DIG-High Prime DNA Labeling and Detection Starter Kit II (Kit for chemiluminescent detection with CSPD, Roche Co.) following the manufacturer's protocol.

Flowering delay, biomass and yield studies: Control and T₀ plants were compared regarding plant height, number of leaves produced before flowering, leaf area, days to flowering after transferring to the greenhouse, mean biomass fresh and dry weight, seed yield and thousand seed weight. The experimental design was a completely randomized design with four replications. Data were analyzed using MSTAT-C software (Freed and Eisensmith, MSTAT-C; A Softward Package for the Design, Management, and Analysis of Agronomic Experiments. East Lansing, Mich.: Michigan State University; (1989)). Means were separated using Tukey's test at the 1% level. T₁ plants were only compared for the delay in flowering. Segregation analysis: T₁ generation seeds were obtained from self-pollination of the T₀ of the five putative transgenic lines. The segregation of the T₁ progeny was tested by culturing 40 seeds of each line on half-strength basal MS medium containing 5 mg/L glufosinate ammonium. Numbers of germinated and non-germinated seeds were recorded after 2 weeks. The chi square (χ²) test at P=0.01 was performed to determine if the observed segregation was consistent with a Mendelian ratio.

Identification of transgenic FLC tobacco plants: Polymerase chain reaction: In addition to the plasmid, a band of the expected size of 338 bp revealed five independent transformation events. No band was detected in the untransformed control (data not shown).

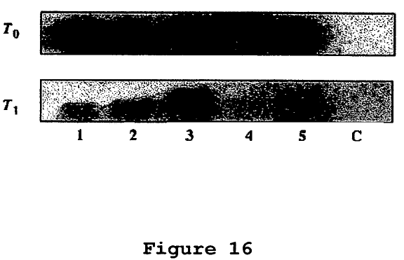

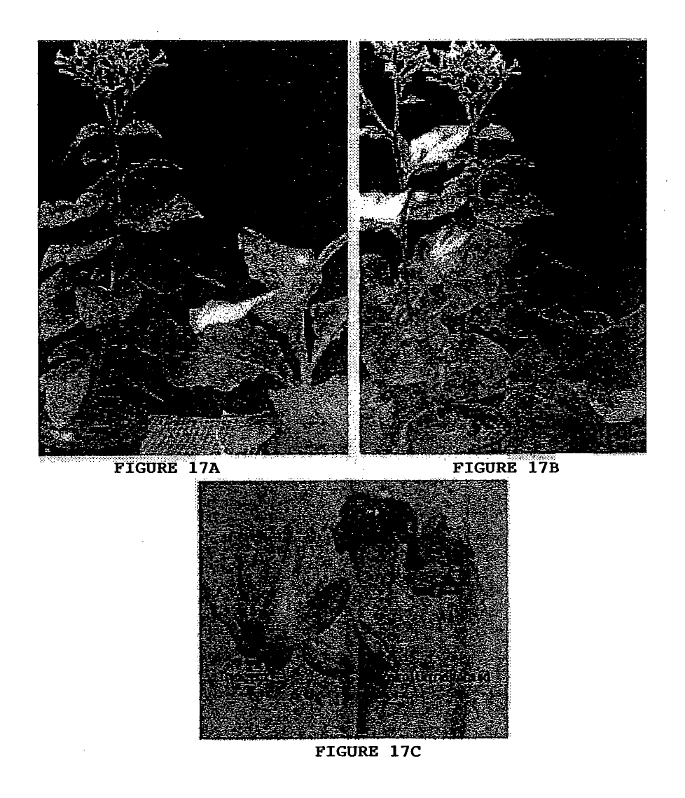

RNA gel blot analysis: The levels of FLC mRNA transcripts in all the transgenic lines for T₀ were high but varied in T₁ (FIG. 16). The results also showed the lack of detectable transcript for FLC in the control plants. FIG. 16 is an illustration of the RNA gel blot analysis of FLC in T0 and T1 tobacco plants. Lanes 1-5 are transgenic lines; Lane C is a negative control.

Flowering delay, biomass and yield studies: Analysis of flowering time of the T₀ transgenic lines and the control yielded informative results about the functionality of FLC in flowering delay. All transformants produced visible flowers later than the control plants with an average of 7 (Line 1) to 36 (Line 4) d with an overall mean of 14 d for all lines (Table 5 and FIG. 17A). This observation was confirmed by anthesis time, as the mature anthers of the control shed pollen though the T₀ lines were still immature (FIG. 17). While there was no significant difference between the five lines and the control regarding number of leaves produced before flowering (Table 5, FIG. 17B), there was significant difference between four lines (lines 1, 3, 4 and 5) and the control regarding plant height at flowering time (Table 5). All the transgenic lines produced leaves larger than the control plants (Table 5). Preliminary experiments with T₁ plants showed more than 4 weeks flowering delay in Line 4. However, plants in the other transgenic lines began flowering about 2 weeks before control.

TABLE 5
Comparison between control and T₀ tobacco plants with regard
to flowering delay and vegetative growth before flowering.

| | Days to | | | | |
|---|---|---|---|---|---|
| | flowering | | No. of | | Plant |
| | after | | leaves | | height |
| | transfer | | produced | Leaf | at |
| | to | Flowering | before | area^{a} | flowering |
| Plants | greenhouse | delay (d) | flowering | (cm²) | time (cm) |
| Control | 15 c^{b} | 0 c | 19 ab | 187.5 d | 60 a |
| T₀: | | | | | |
| Line 1 | 22 b | 7 b | 18 ab | 213.8 c | 35 b |
| Line 2 | 23 b | 8 b | 20 a | 236.5 bc | 65 a |
| Line 3 | 24 b | 9 b | 21 a | 239.5 b | 35 b |
| Line 4 | 51 a | 36 a | 16 b | 369.7 a | 22 c |
| Line 5 | 23 b | 8 b | 20 a | 219.9 bc | 40 b |
| Overall | 29 | 14 | 19 | 256 | 39 |
| mean of | | | | | |
| five lines | | | | | |

^{a}Measured with second fully expanded leaf from the bottom.
^{b}In each column; means followed by the same letters are not significantly different using Tukey's test at 1% level.

FIG. 17. FLC transgenic tobacco plants delayed flowering 2 or more weeks. FIG. 17A: Right plant is FLC transgenic and left plant is untransformed control. FIG. 17B: Plants from Line 4 (right) compared to control plants (left). FIG. 17C: FLC transgenic versus control flowers from the same age. (A=anther, S=stigma). Note the pollen grains on control anthers and stigma.

The phenotype of Line 4 showed the most extreme flowering delay (approximately 5 weeks). Also, due to its short internodes, this line showed a statistically significant short stem length before flowering. Moreover, this line produced the largest leaves among all the other lines and the control (Table 5). There was a significant difference between biomass fresh weight of three lines (lines 2, 4 and 5) and the control but only Line 4 had more biomass dry weight compared to the control (Table 6). With exception of Line 2, all other lines produced lower seed yield compared to the control but the thousand seed weight of all transgenic lines was significantly more than the control plants (Table 6). Line 4 produced the lowest seed yield but the seeds were bigger and heavier (Table 6).

TABLE 6
Differences between control and T₀ tobacco plants in
biomass, thousand seed weight and seed yield per plant

| | | | Thousand | Seed |
|---|---|---|---|---|
| | Biomass | Biomass | seed | yield/ |
| | FW/plant | DW/plant | weight | plant |
| Plants | (g) | (g) | (mg) | (g) |
| Control | 192.31 c^{a} | 35.22 bc | 66 c | 6.19 b |
| T₀: | | | | |
| Line 1 | 233.33 c | 33.47 c | 76 b | 3.18 d |
| Line 2 | 338.52 ab | 50.48 ab | 83 b | 7.12 a |
| Line 3 | 260.81 bc | 35.58 c | 81 b | 5.31 c |
| Line 4 | 335.03 ab | 51.65 a | 101 a | 0.17 e |
| Line 5 | 346.06 a | 45.79 abc | 81 b | 3.36 d |
| Overall mean of five lines | 377 | 43 | 84 | 4 |

^{a}In each column, means followed by the same letters are not significantly different using Tukey's test at 1% level.

Expression of bar gene in the T₁ progeny: A segregation ratio of 3:1 was obtained from all the transgenic lines (data not shown). None of the non-transformed seeds germinated on the selection medium.

Discussion

FLC acts to prevent premature flowering in Arabidopsis (Koornneef et al., Plant J., 6: 911-919 (1994); Lee et al., Plant J., 6: 903-909 (1994); Michaels and Amasino, ibid, 1999; Sheldon et al., ibid, 1999), and was shown to have a similar function when expressed in rice (Tadege et al., Plant Biotechnol J., 1: 361-369 (2003)) and in Brassica napus (Tadege et al., Plant J., 28: 545-553 (2001)), and here, when expressed in tobacco. In Arabidopsis, FLC delays flowering by suppressing the ‘floral pathway integrators,’ including SUPRESSOR OF OVEREXPRESSION OF CO 1 (SOC1) (Henderson and Dean, ibid, 2004; Michaels and Amasino, ibid, 1999 and Michaels and Amasino, ibid, 2001; Sheldon et al., ibid, 1999). Similarly, when expressed in rice, FLC was shown to delay flowering by down-regulating rice SOC1 (Tadege et al., ibid, 2003), causing a similar flowering time effect. In addition, FLC caused reduced fertility and even sterility due to lack of pollen shed in transgenic rice, suggesting that expression of FLC could interfere with other elements of reproductive development (Tadege et al., ibid, 2003). Interestingly, the extended vegetative phase observed when FLC was expressed constitutively in tobacco seems to be caused by a slower rate of leaf initiation (i.e. nodes/day) rather than by a change in the flowering mechanism itself. Transgenic leaves and floral organs were significantly larger than control. It has been suggested that in this case, FLC causes the misdistribution of apical cells into leaf anlagen instead of internodes (S. van Nocker, personal communication). One line (Line 4) had significantly reduced internode length compared with other lines and the control, creating an almost rosette-like phenotype. This is interesting, as FLC comes from Arabidopsis, a rosette plant, while tobacco is not. Perhaps in tobacco, FLC also plays a role in determining internode length, in addition to its role in distribution of apical cells. This would indicate that the function of FLC differs depending on the species it is introduced to.

Delay in flowering time is relative to the level of FLC activity in Arabidopsis and rice: when FLC was introduced under the control of the CaMV 35S promoter in Arabidopsis and under the maize ubiquitin promoter in rice, a wide range of flowering times was observed (Michaels and Amasino, ibid, 1999; Sheldon et al., ibid, 1999; Tadege et al., ibid, 2003), indicating that transgene copy number and/or position effects could greatly affect flowering time (Michaels and Amasino, ibid, 1999). In rice it was shown that the higher the expression of FLC, the greater the delay in flowering and the more severe the floral defects and infertility (Tadege et al., ibid, 2003). In the present work, the delay in flowering time of T₀ plants was 7-49 d, with an overall mean of 14 d. This is more or less consistent with the previous results in Arabidopsis and rice. One line (Line 4) showed up to 49 d (with mean of 36 d) flowering delay.

In the T₀, all the transgenic lines showed a similar level of FLC mRNA expression, with Line 4 showing a slightly higher level. However, our results in the T₁ seem to suggest an opposite relationship: the lower the level of FLC transcripts, the greater the delay in flowering. This is especially true for Line 4 which has the most severe phenotypic effect among all transgenic lines, yet the lowest amount of FLC transcripts in the T₁ generation. Further experiments must be conducted to determine the reasons behind these phenomena in second and further generations.

The delay in flowering resulted in higher biomass yield in all the transgenic lines that was significantly different from control. This higher biomass yield along with production of larger leaves would be useful for efficient production of tobacco, leafy vegetables, and biomass crops such as switchgrass and maize.

Comparing stages of flower development at anthesis of plants that were of the same age, control flowers were completely mature and shed pollen while T₀ plants were still immature (FIG. 17C).

This study shows that expression of FLC in biomass crops may have the desired effect of extending the vegetative stage, with the added benefit of larger leaves and fresh weight biomass. A major problem in maize and other transgenic open-pollinated crops is concern about the movement of transgenes via pollen flow (Riegel et al., Science, 296: 2386-2388 (2002)). Therefore, delay in flowering may not only increase biomass production, but reduce the likelihood of unintended cross-pollination between genetically modified and native cross-breedable plants in the field as their flowering times would be less likely to coincide.

### EXAMPLE 11

This Example relates to Expression of Flowering Locus C in an Acidothermus cellulolyticus E1 endo-1,4-β-Glucanase-Producing Transgenic Tobacco (Nicotiana tabacum L.) and its Effect on Delay in Flowering and Increase in Biomass.

With a hypothesis that delay in flowering increases the biomass of an industrial enzyme-producing transgenic plant, the T₄ generation of an Acidothermus cellulolyticus E1 endo-1,4-β-glucanase-producing transgenic tobacco (Nicotiana tabacum L.) was used for Agrobacterium-mediated transformation and expression of FLOWERING LOCUS C (FLC), a gene from Arabidopsis thaliana that acts as a flowering repressor. Agrobacterium strain GV 3101 containing the FLC and bar selectable marker cassettes was employed for transformation. Six putative transgenic lines, resistant to 5 mg l⁻¹ glufosinate ammonium, were selected for molecular analyses. Genomic DNA and total RNA were isolated from leaves of 6 week old greenhouse plants and used for polymerase chain reaction (PCR) and RNA-blot analysis. The DNA and RNA tests respectively confirmed the integration and transcription of FLC in all six E1cd-FLC lines. Transgenic E1cd-FLC plants showed an average of 9 to 21 d delay in flowering time compared to E1cd-expressing control plants, and the overall mean for all lines was 14 d. E1cd-FLC transgenic plants also displayed significant increases in leaf size and biomass yield. All transgenic E1cd-FLC plants were significantly shorter than control at flowering time. The enzymatic activity of E1cd in the E1cd-FLC expressing plants was similar to the E1cd enzymatic activity in E1cd transgenic control plants. Delay in flowering of transgenic plants could be a useful bioconfinement system to avoid or reduce the chance of cross contamination of transgenic pollen with cross-breedable plants in the field, and the increase in biomass could be a useful trait to increase production of biomass per plant.

Air pollution and global warming as a result of burning fossil fuels, and the high costs of fossil fuel have compelled researchers to develop alternative energy sources such as lignocellulosic biomass (Ziegelhoffer et al., ibid, 2001). Cellulases are a class of enzymes with great potential for bioconversion of lignocellulosic biomass to ethanol and other important industrial chemicals (Wright, Energy Prog. 8:71-78 (1988); Lynd et al., Science, 251: 1318-1323 (1991); Halliwell and Halliwell, Outlook Agric. 24: 219-225 (1995)). However, the high cost of cellulase enzyme production in bacterial fermentation tanks is a barrier to the utilization of these enzymes at commercial level (Ziegelhoffer et al., ibid, 1999). Technology to produce hydrolysis enzymes in transgenic crops may become very valuable in reducing these costs (Teymouri et al., Appl. Biochem. Biotechnol. 116: 1183-1192 (2004)). To test whether plants could produce biologically active microbial cellulases, Arabidopsis thaliana (Ziegler et al., Mol. Breed. 6: 37-46 (2000)), tobacco, alfalfa and potato (Ziegelhoffer et al., ibid, 1999) have been genetically engineered with microbial cellulase gene. Also, cellulase-producing transgenic tobacco has been used to test the stability of activity of the herterologues cellulase in plant material after Ammonia Fiber Explosion (AFEX) pretreatment (Teymouri et al., ibid, 2004).

It is well understood that the biomass production decreases after transition from vegetative plant growth (i.e. production of leaves) to a reproductive stage (i.e. production of flowers). Therefore, if the onset of flowering could be delayed, this is assumed to give the plant a longer vegetative growth period resulting in a higher biomass.

Flowering, the transition from the vegetative to the reproductive stage, is a key developmental change in the life cycle of the plant (Simpson and Dean, ibid, 2002; Henderson and Dean, ibid, 2004) and is controlled by both environmental and developmental signals (Jang et al., ibid, 2003). Flowering has been the subject of many studies. However, it is still not well understood at the molecular level due to the complexity of the flower initiation phenomena (Koornneef et al., ibid, 1998).

Henderson and Dean, ibid, (2004) recently presented a model that shows the current understanding of flowering in Arabidopsis. This model represented the participation of at least eight distinct pathways regulating the transition from vegetative growth to reproductive organ development. While these pathways perform largely independent of one another, certain interaction takes place among them (Koornneef et al., ibid, 1998; Rouse et al., ibid, 2002). Several factors, including the light quality, ambient temperature, gibberellin, circadian clock, and photoperiod pathways may promote flowering (Henderson and Dean, ibid, 2004). Acting against these pathways is the floral repressor gene FLOWERING LOCUS C (FLC). A number of genes act to promote FLC expression. It has been shown that FLC is down-regulated by vernalization (i.e. long exposure to near-freezing temperatures) and the autonomous pathway genes (Michaels and Amasino, ibid, 1999; Sheldon et al., ibid, 1999; Sheldon et al., ibid, 2000; Michaels and Amasino, ibid, 2001; Henderson and Dean, ibid, 2004). Here we report an Agrobacterium-mediated transformation of E1cd transgenic tobacco (Ziegelhoffer et al., Mol. Breed. 8: 147-158 (2001)), a cellulase-producing bioreactor plant, with a constitutively regulated FLC, and the molecular and physiological analyses of the transgenic plants.

Materials and Methods

Plant materials. Seeds of T₃ transgenic tobacco (Nicotiana tabacum L.) plants expressing E1cd (catalytic domain fragment of E1 endo-1,4-β-glucanase from Acidothermus cellulolyticus) we used from our previous research (Teymouri et al., ibid, 2004). Initially, the T1 seeds were obtained from Dr. Sandra Austin-Phillips of the University of Wisconsin. In their E1cd transformation research, the team used the pZA9 containing E1cd regulated by the CaMV 35S (Cauliflower Mosaic Virus 35S) promoter, the apoplast-targeting leader VSPβ of soybean, and nopaline synthase terminator (Nos); and used nptII as the selectable marker gene (Ziegelhoffer et al., ibid, 2001).

Seeds of T₃ plants were prewashed in water with 0.2% Tween-20 for 10 min and rinsed three times with distilled water. The seeds were surface sterilized with 70% (v/v) ethanol for 1 min, immersed in 20% (v/v) Clorox (5.25% sodium hypochlorite) for 20 min and then rinsed three times with sterilized double distilled water. Seeds were germinated on Murashige and Skoog (1962) (MS) basal medium (Sigma-Aldrich, St. Louis, Mo.) containing 30 g l⁻¹ sucrose and 2.5 g l⁻¹ gelrite (Sigma-Aldrich, St. Louis, Mo.). Cultures were kept under 30 μmol m⁻² s⁻¹ continuous white deluxe fluorescent light at 25° C. Leaf segments (0.5 cm×0.5 cm squares) were aseptically excised from the second and third fully expanded in vitro produced leaves for infection with Agrobacterium tumefaciens (Horsch et al., ibid, 1985).

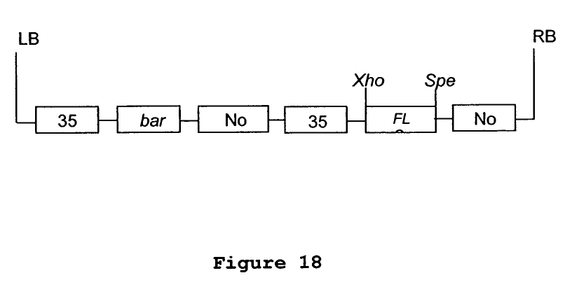

Agrobacterium strain and plasmid. Agrobacterium tumefaciens strain GV 3101 (pMP90RK) (Koncz and Schell, ibid, 1986) containing the 3.232 kb binary vector pGreen (Hellens et al., ibid, 2000a) was employed for transformation experiments. The plasmid contains FLC from Arabidopsis thaliana and phosphoinothricin acetyltransferase gene (bar), both under the control of the cauliflower mosaic virus (CaMV) 35S promoter and the nopaline synthase (nos) terminator (FIG. 18).

The Agrobacterium containing the transgenes was grown in 10 ml YEP medium (containing 10 g l⁻¹ Bacto-peptone, 10 g l⁻¹ Bacto yeast extract, 5 g l⁻¹ NaCl, pH 7.2) supplemented with 25 g l⁻¹ of both kanamycin and gentamycin (Hellens et al., ibid, 2000b), incubated at 28° C. and 250 rpm for 48 h, and the cultures (cell density 0.6-0.8 at A₆₀₀) were used for transformation.

Agrobacterium-mediated transformation. Leaf segments were infected using the Agrobacterium culture at room temperature for 25 min. Then, the leaf explants were blotted on sterilized filter papers and placed upside down on MS medium supplemented with 4.5 μM N 6-benzylamino purine (BAP) and 0.5 μM α-naphthaleneacetic acid (NAA) (Ziegelhoffer et al., ibid, 1999), 30 g l⁻¹ sucrose and 2.5 g l⁻¹ gelrite (co-cultivation medium). The leaf segments were kept in co-cultivation medium for two days under continuous light as described above for seed culture. Then, they were rinsed three times with sterilized distilled water containing 400 mg l⁻¹ carbencillin, blotted onto sterilized filter papers and placed on the same co-cultivation medium supplemented with 400 mg l⁻¹ carbencillin and 5 mg l⁻¹ glufosinate ammonium for selection of the putative transformants. The produced calli were subcultured in the same medium, and then shoots were excised and rooted on half-strength MS medium containing 400 mg l⁻¹ carbencillin and 5 mg l⁻¹ glufosinate ammonium in Magenta boxes (Sigma-Aldrich, St. Louis, Mo.). Well-rooted plantlets were transferred to the greenhouse after acclimatization. Greenhouse conditions were 25 to 28° C., 90-95% relative humidity and 190 μmol m⁻² s⁻¹ light.

Polymerase chain reaction (PCR) analysis. After selection on glufosinate ammonium-containing medium, PCR analysis was used to screen the transgenic plants for FLC and E1cd transgene incorporation. Six independent transgenic lines were selected for PCR screening. Total genomic DNA of control and transgenic plants were extracted from leaves as described by Edwards et al. (1991). The following set of primers were used: FLC F, 5′-CGA TAA CCT GGT CAA GAT CC-3′ (forward primer, SEQ ID NO:25) and FLC R, 5′-CTG CTC CCA CAT GAT GAT TA-3′ (reverse primer, SEQ ID NO:26), and E1cd F, 5′-GCG GGC GGC GGC TAT TG-3′ (forward primer, SEQ ID NO:27) and E1cd R, 5′-GCC GAC AGG ATC GAA AAT CG-3′ (reverse primer, SEQ ID NO:28). The predicted size of the amplified DNA fragments of the transgene was 338 bp for FLC, and 1.07 kb for E1cd. DNA amplifications were performed in a thermo cycler (Perkin Elmer/Applied Biosystem, Foster City, Calif.) using REDTaq™ ReadyMix™ PCR Reaction Mix with MgCl₂ (Sigma-Aldrich, St. Louis, Mo.). The PCR profile had an initial denaturation step at 94° C. for 1 min, followed by 30 cycles of 1 min at 94° C. (denaturation), 2 min at 60° C. (annealing) and 3 min at 72° C. (extension). The reaction mixture was loaded directly onto a 1.0% (w/v) agarose gel, stained with ethidium bromide and visualized with UV light.

RNA-blot analysis. Total RNA of control plants and PCR-positive transgenic plants for both FLC and E1cd from six putative transgenic lines was isolated from leaves of six-week-old greenhouse plants using the TRI Reagent (Sigma-Aldrich, St. Louis, Mo.) according to the manufacturer's instructions. Twenty micrograms of RNA were fractionated in 1.2% agarose formaldehyde denaturing gel and blotted onto a Hybond-N⁺ nylon membrane (Amersham Pharmatica. Biotech.) as specified by the manufacturer. The probe was generated by digesting plasmid DNA with XhoI and SpeI, releasing the 0.59-kb fragment containing the FLC coding region. The digestion reaction mixture was gel-purified using the QIAquick Gel Extraction Kit (QIAGEN Inc., Valencia, Calif.). Probe labeling and transcript detection were obtained using the DIG-High Prime DNA Labeling and Detection Starter Kit II (Kit for chemiluminescent detection with CSPD, Roche Co.) following the manufacturer's protocol.

Flowering delay, biomass and yield studies. Control and E1cd-FLC transgenic plants were compared concerning plant height, number of leaves produced before flowering, leaf area, days to flowering after transferring to the greenhouse, mean biomass fresh and dry weight, seed yield and thousand seed weight. The experimental design was a completely randomized design (CRD) with four replications. Data were analyzed using MSTAT-C software (Freed and Eisensmith, ibid, 1989) and means were separated using Tukey's test at the 1 or 5% level.

Segregation analysis. Segregation analysis was conducted using the T₁ generation seeds of the E1cd-FLC self-pollinated plants of the six putative transgenic lines. Forty seeds of each line were cultured on half-strength basal MS medium containing 5 mg l⁻¹ glufosinate ammonium. Numbers of germinated and non-germinated seeds were recorded after 2 weeks. The chi square (χ²) test at P=0.01 was performed to determine if the observed segregation was consistent with a Mendelian ratio.

E1cd enzymatic activity of E1cd-FLC transgenic plants. Samples of E1cd-FLC and control untransformed plants were assayed for E1 activity as described (Ziegelhoffer et al., ibid, 2001; Teymouri et al., ibid, 2004). Briefly, a standard curve was generated using 4 to 160 pmol 4-methylumbelliferone (MUC), the product of E1 hydrolization of the substrate 4-methylumbelliferone β-D-cellobioside (MUC). Total soluble protein was isolated from 100 mg fresh leaf tissue using the sodium acetate grinding buffer and ammonium sulfate precipitation described in Ziegelhoffer et al., ibid, (1999) and quantified by using the BioRad (Hercules, Calif.) Protein Dye Reagent, measuring the absorbance at 595 nm and comparing the value to the standard curve as specified by the manufacturer. A series of soluble protein dilutions ranging from 10⁻¹ to 10⁻³ were made. In a 96-well plate, 1 to 4 μl sample were mixed with 100 μl reaction buffer containing MUC. Plates were covered with adhesive lids and incubated at 65° C. for 30 minutes. The reaction was stopped and the fluorescence was read at 465 nm using SPECTRAmax M2 device (Molecular Devices Inc., Sunnyvale, Calif.) at an excitation wavelength of 360 nm. After subtracting background fluorescence contributed by the control, activity of samples was calculated using the standard curve and compared to the activity of pure E1 reported in Ziegelhoffer et al., ibid, (1999). Briefly, the nmol MU (from the standard curve) was divided by 30 minutes to calculate nmol MU/min; this number was divided by the μg total protein in the well to calculate the activity.

Vernalization studies. To test the effect of vernalization on delay in flowering, seeds of control untransformed tobacco plants and seeds of E1cd-FLC tobacco were allowed to germinate on wetted filter papers in petri dishes. Petri dishes were kept in the dark at 4° C. (14) for 30 d. Then, the seedlings were planted in the soil and transferred to the greenhouse, where they were grown until flowering.

Results and Discussion

Polymerase chain reaction. PCR analysis showed the integration of both FLC and E1cd in all the lines. The E1cd transgenic control plants showed only presence of E1cd (data not shown).

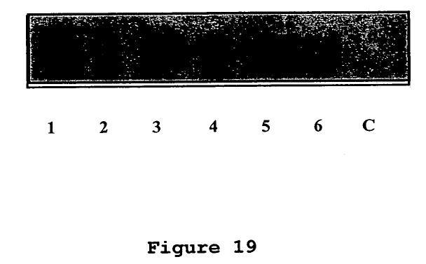

RNA-blot analysis. All the transgenic lines showed high levels of FLC mRNA transcripts (FIG. 19). No band was detected for control plants. Based on PCR and RNA-blot analyses, transformation and selection efficiency can be estimated as 100%.

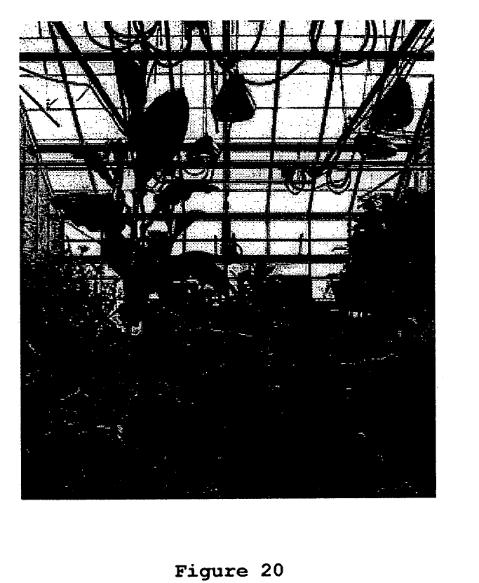

Flowering delay, biomass and yield studies. Control plants flowered 23 days after transfer to the greenhouse (Table 7). Transgenic plants showed 32 to 44 days of vegetative growth before to switching to the reproductive stage, after transfer to the greenhouse (Table 7). Therefore, transgenic plants showed a delay in flowering of 9 to 21 days, with a mean of 15 days greater control plants (Table 7). This is more or less consistent with the previous results in Arabidopsis (Koornneef et al., ibid, 1994; Lee et al., ibid, 1994; Michaels and Amasino, ibid, 1999; Sheldon et al., ibid, 1999), Brassica napus (Tadege et al., The Plant Journal, 28: 548-553 (2001)) rice (Tadege et al., ibid, 2003) and our previous study on tobacco (Salehi et al., J. Plant Physiol., 162: 711-717 (2005)). Lines 1 and 4 showed the greatest delays in flowering, 18 to 25 days, with a mean of 21 days, and 17 to 26 days, with a mean of 20 days, respectively. FLC is known to prevent premature flowering in Arabidopsis (Koornneef et al., ibid, 1994; Lee et al., ibid, 1994; Michaels and Amasino, ibid, 1999; Sheldon et al., ibid, 1999), Brassica napus (Tadege et al., ibid, 2001) rice (Tadege et al., ibid, 2003) and tobacco (Salehi et al., ibid, 2005). In our experiment, all the transgenic lines were shorter than control at flowering time, with no significant difference in nodes or leaf number (Table 7, FIG. 20). Practically speaking, the shorter stem should be one the advantages of biomass plants considering normal lodging or stem breakage in the field. Lines 1 and 4 produced leaves significantly larger than control and other lines (Table 7, FIG. 20).

TABLE 7
Comparison between control and E1cd-FLC transgenic tobacco plants WITH
REGARD TO flowering delay and vegetative growth before flowering.

| | Days to | | | | Plant |
|---|---|---|---|---|---|
| | flowering after | | No. of leaves | | height at |
| | transferring to | Flowering | produced before | Leaf area* | flowering |
| Plants | the greenhouse | delay (d) | flowering | (cm²) | time (cm) |
| Control | 23 b | 0 b | 20 a | 333.5 c | 113.3 a |
| Transgenic: | | | | | |
| Line 1 | 44 a | 21 a | 19 a | 518.0 a | 50.0 b |
| Line 2 | 39 a | 16 a | 20 a | 218.5 e | 56.8 b |
| Line 3 | 32 ab | 9 ab | 19 a | 345.8 c | 45.5 b |
| Line 4 | 43 a | 20 a | 19 a | 477.5 b | 47.0 b |
| Line 5 | 38 b | 15 a | 21 a | 239.5 de | 48.0 b |
| Line 6 | 35 ab | 12 ab | 20 a | 253.0 d | 54.5 b |
| Overall | 38 | 15 | 20 | 342 | 50 |
| mean of | | | | | |
| six lines | | | | | |

*Measured with second fully expanded leaf from the bottom.
In each column, means followed by the same letters are not significantly different using Tukey's test at P ≦ 0.01.

Lines 1 and 4 had significantly more biomass fresh weight than all four other lines (P≦0.01) and the control (P≦0.05) (Table 8). Biomass dry weight was more or less the same in control and all the transgenic lines (Table 8). The higher fresh biomass yield along with production of larger leaves might be useful for more cellulase production from E1cd-FLC tobacco plants.

TABLE 8
Differences between control and E1cd-
FLC transgenic tobacco plants in biomass,
thousand seed weight and seed yield per plant

| | Biomass | | | |
|---|---|---|---|---|
| | FW/plant | Biomass | Thousand seed | Seed |
| Plants | (g) | DW/plant | weight (mg) | yield/plant |
| Control | 187.0 abc | 32.75 a | 658 c | 5.51 abc |
| Transgenic: | | | | |
| Line 1 | 275.3 ab* | 29.25 ab | 701 bc | 4.90 bcd |
| Line 2 | 181.8 bc | 30.50 ab | 740 ab | 3.87 cd |
| Line 3 | 164.3 c | 21.00 b | 792 a | 3.75 d |
| Line 4 | 291.3 a* | 35.50 a | 745 ab | 6.89 a |
| Line 5 | 159.8 c | 19.25 b | 726 b | 4.01 cd |
| Line 6 | 179.5 bc | 27.00 ab | 588 d | 6.30 ab |
| Overall mean of | 209 | 27 | 715 | 5 |
| six lines | | | | |

In each column, means followed by the same letters are not significantly different using Tukey's test at P ≦ 0.01.
*Significantly different from control using Tukey's test at P ≦ 0.05.

Except line 3, seed yield per plant was not significantly different between control and transgenic plants. Also, except line 6, the thousand seed weight of all transgenic lines was significantly more than the control plants (Table 8). Line 3 produced the lowest seed yield but the seeds were larger and heavier (Table 8). In transgenic rice, FLC caused reduced fertility and even sterility, suggesting that expression of FLC could get in the way with other elements of reproductive development (Tadege et al., ibid, 2003). In transgenic rice, FLC caused reduced fertility and even sterility, suggesting that expression of FLC could get in the way with other elements of reproductive developments (Tadege et al., ibid, 2003), that we believe has been due to the transgene position effect rather than the transgene physiological effect. In our case, except line 3, FLC did not reduce the fertility, and transgenic plants produced seed weight the same amount as the control plants (Table 8).

Expression of bar gene in the T₁ progeny. Seeds of all the E1cd-FLC transgenic lines were germinated on selection medium with a segregation ratio of 3:1 (Table 9). None of the control non-transformed seeds germinated on the selection medium.

TABLE 9
Segregation of glufosinate ammonium resistance (germinated
vs. non-germinated seeds) in E1cd-FLC T₁ progeny*

| | | Number of | | |
|---|---|---|---|---|
| | Number of | non- | | |
| | germinated | germinated | Expected | |
| Lines | seeds | seeds | ratio | χ² |
| 1 | 30 | 10 | 3:1 | 0.000^{ns} |
| 2 | 27 | 13 | 3:1 | 1.999^{ns} |
| 3 | 30 | 10 | 3:1 | 0.000^{ns} |
| 4 | 32 | 8 | 3:1 | 0.533^{ns} |
| 5 | 31 | 9 | 3:1 | 0.133^{ns} |
| 6 | 29 | 11 | 3:1 | 0.133^{ns} |

*Forty seeds were used for each line.
^{ns}Non-significant.

Enzymatic activity of E1cd in E1cd-FLC transgenic plants. According to Ziegelhoffer et al., ibid, (2001), E1 in E1cd plants hydrolyzed 4-methylumbelliferone β-D-cellobioside (MUC) to 4-methylumbelliferone (MU) at a rate of 40 nmol of substrate per microgram per minute. The enzymatic activity of E1 enzyme extracted from apoplast-targeted transgenic E1cd which were further transformed with FLC (so called E1cd-FLC) was 1.4726 nM/μg/min. This activity is similar to the E1 enzymatic activity that was originally reported by Ziegelhoffer et al. ibid, (2001) and confirmed by Teymouri et al. ibid, (2004) for earlier E1cd transgenic generations of these plants. This confirms that addition of FLC does not affect E1cd enzymatic activity.

Vernalization studies. As tobacco is an annual and a warm-season crop, it normally does not require vernalization to induce flowering. However, because vernalization has the ability to down-regulate FLC expression in Arabidopsis, the FLC-expressing transgenic plants were tested for vernalization effects in case of accidental cold exposure. As expected, vernalization had no effect on flowering time, which is consistent with a similar experiment in Brassica napus (Tadege et al., ibid, 2001). Vernalization is downstream of FLC, and because the CaMV35S promoter is a strong promoter, it would overturn any effects vernalization may have had on FLC, and as pointed out previously (Tadege et al., ibid, 2001), it is not cold-responsive.

### EXAMPLE 12

This Example illustrates the High-Level Production of Endo-1,4-β-Glucanase in Transgenic Rice with Subsequent Enhanced Conversion of Biomass Polysaccharides into Fermentable Sugars.

The catalytic domain of Acidothermus cellulolyticus thermostable endoglucanase gene (encoding for endo-1,4-β-glucanase enzyme or E1) was constitutively expressed in rice using the Agrobacterium-mediated transformation system in an apoplast-targeting manner. Molecular analyses of T1 plants confirmed presence and expression of the transgene. The amount of E1 enzyme accounted for up to 4.9% of the plant total soluble proteins, and its accumulation had no apparent deleterious effects on plant growth and development. Approximately 22 and 30% of the cellulose in the Ammonia Fiber Explosion (AFEX)-pretreated rice and maize biomass was converted into glucose using rice E1 heterologous enzyme respectively. As rice is the major food crop of the world with minimal use for its straw, the results may suggest a successful strategy for producing biologically active hydrolysis enzymes in rice to help generate alcohol fuel while substituting the wasteful and polluting practice of rice straw burning with an environmentally superior technology.

The fuel ethanol industry has been growing extensively in many countries worldwide (Renewable Fuels Association. Homegrown for the homeland: Industry Outlook Report 2005. p. 14 (2005)), and considerable efforts have been exerted towards improving ethanol yield and reducing its production costs during the last two decades (Ingledew, W. M. In: T. P. Lyons, D. Kelsall & Murtagh J. Editors. The Alcohol Textbook Nottingham University Press, Nottingham, UK, 55-79 (1995) and Lynd, L. R., et al, Consolidated bioprocessing of cellulosic biomass: an update. Curr. Opin. Biotechnol. 16, 577-583 (2005)). A vision to enhance U.S. economic security has set a target of using plant-derived materials to meet 10% of chemical feedstock demand by 2020-a fivefold increase (Singh, S. P. et al, International Food and Agribusiness Management Review 5, 1-15 (2003). In 2004, U.S. ethanol production capacity reached 3.535 billion gallons, about 303 million gallons more than 2003. Another production increase of more than 500 million gallons is projected for 2006 (MacDonald, T. et al, California Energy Commission Report. P. 6 (2003)). Until now, the vast majority of U.S. ethanol has been produced from maize seeds (Renewable Fuels Association, ibid, 2005), while Brazil produces similar quantities of ethanol from sugar derived from cane. The economic and environmental performance of maize and sugar ethanol would likely be improved by producing ethanol from lignocellulosic materials instead. Approximately 1.3 billion tons of crop and forest residues and energy crops are thought to be available in the United States with proper management. The energy value of this much lignocellulosic biomass is roughly equivalent to 3.5 billion barrels of petroleum per year; the total amount of petroleum produced in the United States in its peak production year-1972. Worldwide, over 1.7 billion tons of crop residues are available annually (Kim, S. and Dale, B. E. Biomass and Bioenergy. 26, 361-375 (2004)) and nearly half of this total is rice straw. Energy crops could add many more billions of tons of lignocellulosic biomass for processing to fuels and chemicals. A recent comprehensive study prepared under the leadership of the Natural Resources Defense Council highlights the potential economic and environmental benefits of very large scale conversion of lignocellulosic biomass to ethanol, electricity and other fuels (Greene, N., Growing Energy: How Biofuels Can Help End America's Oil Dependence. Natural Resources Defense Council. December (2004)).

For ethanol to be produced from plant biomass sources, enzymatic hydrolysis of cellulose to fermentable sugars is employed (Kabel, M. A. et al, Biotechnol. Bioeng. 93, 56-63 (2005)) using hydrolysis enzymes (Ziegler, ibid, 2000 and Ziegelhoffer, ibid, 2001). These enzymes are produced in large-scale microbial fermentation tanks (Howard, R. L. et al, Afr. J. Biotechnol. 2, 602-619 (2003) and Knauf, M. et al., Internat Sugar Jour. 106, 147-150 (2004)). Although the cost of enzyme production was reduced by about a factor of four from 1980 to 1999 (Wyman, C. E., Annu. Rev. Energ. Env. 24, 189-226 (1999)) and by another 10 fold since 2000 (Knauf, M., ibid, 2004), it still represents about $0.20 per gallon of lignocellulosic ethanol-a major cost factor. However, cost reduction might be achieved by producing crops that can sustainably and actively self-produce the desired hydrolysis enzymes (Sheehan, J. et al, Biotechnol. Progr. 15, 817-827 (1999) and Teymouri, F. et al, ibid, 2004).

In addition, the specific enzyme mixture was developed for corn stover treated with dilute acid and it is yet to be demonstrated whether or not the specific enzyme mixture will be suitable for other biomass materials or other pretreatments. In biological conversion of biomass to ethanol, the biomass raw material, the pretreatment and hydrolysis enzymes used after pretreatment to produce sugars must function together as a system (Wyman, C. E. et al, Bioresource Technology. 96, 1959-1966 (2005)). Enzymes developed for acidic pretreatments are likely not suitable for pretreatments using neutral or alkaline conditions. One set of high value hydrolysis enzymes that might be alternatively produced within the biomass crops and utilized in fuel ethanol production in a biorefinery are the “cellulases”—enzymes that convert cellulose into fermentable sugars.

The E1 endo-1,4-β-glucanase enzyme of A. cellulolyticus is one of the most thermostable cellulases known (Baker, J. O. et al, Appl Biochem. Biotechnol. 45/46, 245-256 (1994)). The expression of E1cd endo-1,4-β-glucanase in tobacco (Ziegelhoffer et al, ibid, 2001) potato (Dai, ibid, 2000), and Arabidopsis (Ziegler, ibid, 2000) plants demonstrated the possibility of producing this enzyme in plants. Several prominent crops have been recommended for this purpose, especially those with a high-lignocellulosic biomass, which some presently cause disposal problems (Knauf, ibid, 2004 and Sticklen, M. et al, 2^{nd} International Ukrainian Conference on biomass for energy, p. 133, 20-22 September 2004, Kyiv, Ukraine (2004)).

Rice, as the primary source of caloric intake for over half of the world's human population, is grown on over 148 million hectares worldwide (Chandra Babu, R., et al. Crop Sci. 43, 1457-1469 (2003)) with a total production of about 800 million tons of straw (Jiang, J. et al, Crop Sci. 40, 1729-1741 (2000)). Because of its central role in food supply, significant advances have already been made in the development of rice genetic transformation methods and incorporation of genes conferring important agronomic traits (Jiang, J. et al, ibid, 2000).

While rice seed is the traditionally useful portion of this important crop, its remaining biomass has to date shown limited use. Traditionally, farmers throughout the world burn rice straw in the field after harvest. Burning is inexpensive and mitigates rice diseases. However, emitted smoke give rise to health concerns such as increased incidence of asthma (McCurdy, S. A., et al., Am. J. Respir. Crit. Care Med. 153, 1553-1559 (1996); Jacobs, J. et al, Environ. Health Perspect. 105, 980-985 (1997); Torigoe, K. et al, Pediatr. Int. 42, 143-150 (2000); Golshan, M. et al, Int. J. Environ. Health Res. 12, 125-131 (2002) and Kayaba, H. et al, Tohoku J. Exp. Med. 204, 27-36 (2004)), among others. These concerns, for example, resulted in California legislation that limits rice straw burning to the lesser of 125,000 acres or 25% of rice area in 2001, and even then burning is allowed only if evidence of disease is present (California Rice Commission, http://www.calrice.org/ala_burning.htm). California harvested 508,000 acres of rice in 2005, down from a peak of 590,000 acres in 2004 but nearly ten percent higher than the 465,000 acres harvested a decade earlier (USDA-National Agricultural Statistical Service, http://www.nass.usda.gov/QuickStats/). As a result, California produces an excess of one million tons of rice straw each year. Of the rice straw produced in California, only about 3-4% is used in commercial applications (http://www.ethanolmarketplace.com/061305_news2.asp) and the rest must be incorporated into the soil by plowing and adding water to aid decomposition. The cost of this soil incorporation is about $43/acre, for a total of $15-18 million per year (California Rice Commission, Rice Straw, http://www.calrice.org/a5a_recycling.htm).

Therefore, rice may become a viable bio-based energy candidate with potential to lower pollution levels at the same time. Moreover, the production of enzymes in rice straw may prove fruitful for the manufacture of other valuable bio-based industrial enzymes and protein pharmaceuticals.

The following study examines the production of biologically active A. cellulolyticus endo-1,4-β-glucanase E1 enzyme in transgenic rice plants and conversion of crop biomass cellulose-to-glucose using rice-produced E1 heterologous enzyme.

Results

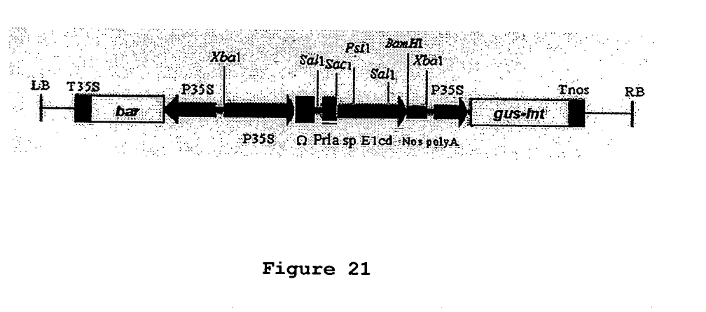

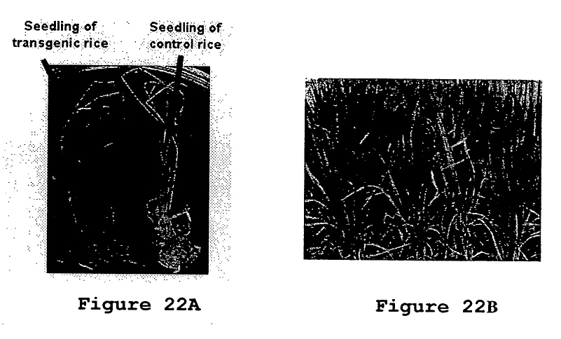

Construct and genetic transformation. The ApoE1 binary vector (FIG. 21) containing the catalytic domain of the A. cellulolyticus thermostable endoglucanase (E1) gene (encoding for endo-1,4-β-glucanase enzyme) was introduced into the nuclear genome of mature embryo-derived calli of the rice variety Taipei 309 (Oryza sativa L. subsp. Japonica) using the Agrobacterium-mediated transformation system (Ahmad, A. et al, In Vitro Cell. Devel. Biol. 38, 213-220 (2002) and Cheng, M. et al, In Virto Cell. Dev. Biol. 40, 31-45 (2004)). Transformation frequency, as defined in terms of percentage of glufosinate herbicide resistant calli was 32%. About 78% of these glufosinate-resistant embryogenic calli differentiated into plantlets in the presence of 15 mg/L glufosinate ammonium. Many transgenic plants were produced among which five independent transgenic events were selected for further molecular and biochemical analysis. T0 and T1 plants grew well under controlled environments with no apparent growth or developmental abnormalities (FIG. 22B).

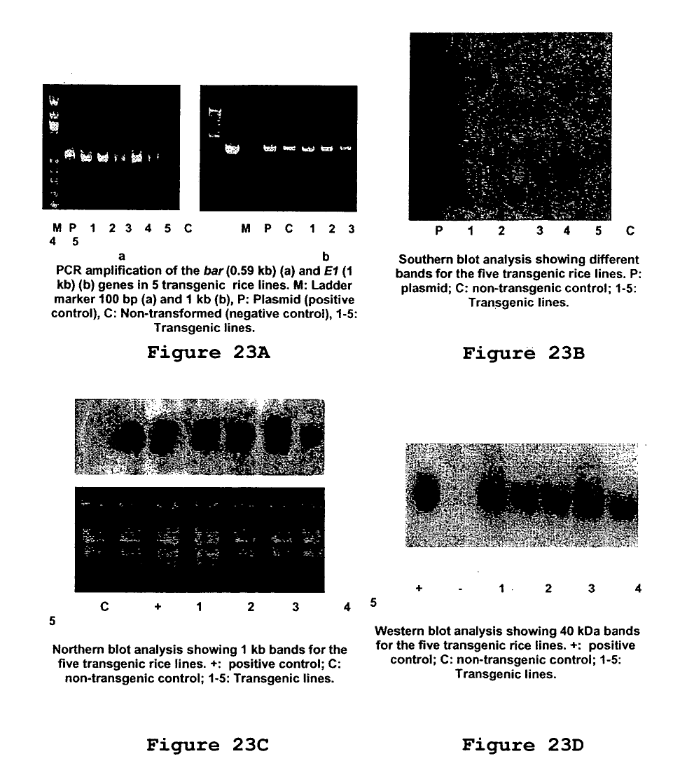

Molecular analysis of the transgenic plants. Combining the results of stable GUS expression patterns (for the gus gene) and PCR (for the bar and E1 genes) assays confirmed the presence of intact, linked gus, bar and E1 genes. The blue color of GUS expression patterns were observed in the transgenic plantlets (FIG. 22A). The expected PCR bands (0.59 kb for bar and 1 kb for E1) were confirmed in the plasmid and the transgenic rice lines, but not in the untransformed control plants (FIG. 23A).

Southern blot analysis confirmed the stable incorporation, copy number and independence of the transgenic lines (FIG. 23B). The genomic DNA of the five transgenic lines showed bands of different sizes, as an indication of five independent transgenic events with 1-2 copies.

When Northern blot analysis was used to confirm the transcription of the E1 gene, a transcript of approximately 1 kb for this gene was detected in the transgenic tobacco positive control (tobacco transformed with the same construct) as well as the rice transgenic lines, indicating that the transgenic lines possess the transcriptionally-active E1 gene (FIG. 23C).

Western blot analysis of leaf total soluble proteins (LTSP) using the mouse antibody raised against the E1 protein confirmed the expression of E1 both in transgenic rice and transgenic tobacco positive control, with the expected molecular mass of 40-kDa (FIG. 23D). Furthermore, the relative amount of transcript and 40 kDa E1 polypeptide in all five transgenic lines, judged from band intensity respectively in Northern and Western blots, correlated well with the amount of E1 produced in the transgenic lines using the 4-methylumbelliferyl β-D-cellobioside assay (MUCase) (FIGS. 23C,D and Table 10).

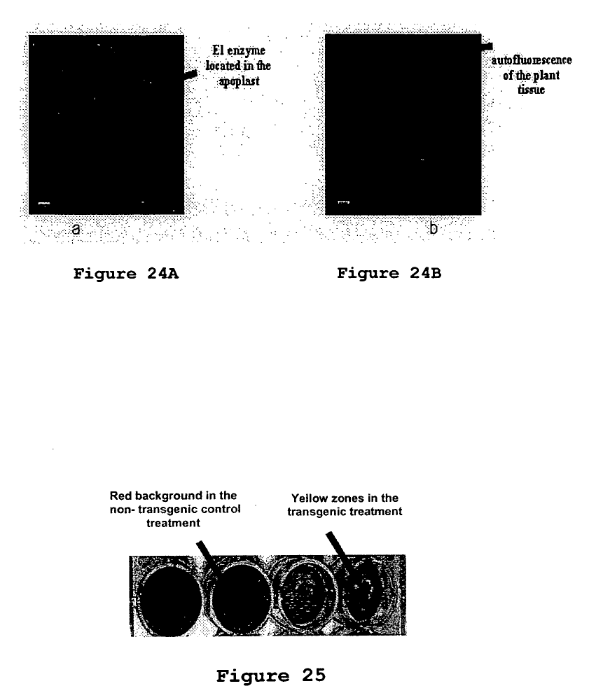

Localization of the E1 enzyme in the apoplast. Strong green fluorescent signals were detected in the apoplast of the transgenic tissues upon the application of immunoflouresence scanning laser confocal microscopy, confirming accumulation of the E1 enzyme. No signals were detected in the untransformed control plant tissues (FIG. 24).

High-level production of biologically active E1 enzyme. E1 enzyme was expressed at relatively high levels of 2.4-4.9% of LTSP, as detected among the transgenic lines. The carboxymethyl cellulase activity assay (CMCase) confirmed that the rice-produced heterologous E1 is biologically active. In this confirmation, zones of carboxymethylcellulose (CMC) hydrolyzed by the enzyme were decolorized with a washing buffer, leaving yellow regions in the transgenic as compared with red background in the control untransformed plant samples (FIG. 25). The results suggest that the microbial E1 enzyme remained biologically active in the transgenic rice plants while E1 activity was not present in the untransformed plants (Table 10).

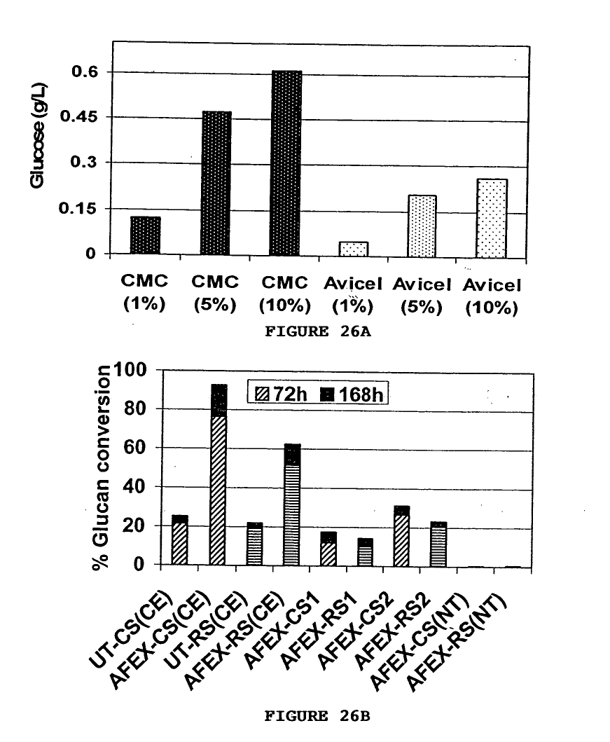

Glucan to glucose conversion. The Ammonia Fiber Explosion (AFEX)-pretreated (Teymouri, F. et al, ibid, 2004) maize and rice biomass (lignocellulosic substrates containing both amorphous and crystalline cellulose), as well as increasing concentrations of both CMC (amorphous cellulose) and Avicel (crystalline cellulose) were converted into glucose using the transgenic rice plant total soluble proteins containing the E1 enzyme. Using 10% CMC and Avicel concentrations, approximately 0.6 and 0.2 g/L glucose was released after 168 hour of hydrolysis, respectively (FIG. 26A). Additionally, considerable amounts of polyoligosaccharides were released from the CMC substrate blank, and the apparent solution viscosity increased substantially. Conversely, when an aliquot of rice E1-containing total soluble proteins was added to the CMC substrate, viscosity declined with reduced polyoligosaccharide formation and a detectable increase in the glucose peak (results not shown).

Approximately 25% and 95% glucan conversion was achieved for untreated and AFEX-treated corn stover respectively, when the cellulase commercial enzyme (Spezyme CO, Genencore) along with β-glucosidase (Novo 188, Sigma) was used in each case. Under similar conditions, untreated and AFEX-treated rice straw showed 21% and 62% glucan conversion respectively. Since both untreated corn stover and rice straw showed much lower conversion compared to AFEX-treated biomass (less than 2% using E1-containing rice extract and 25% and 21% using cellulase commercial respectively), AFEX-treated biomass was used for further experiments. When 0.5 ml of rice extract containing 4.9% LTSP E1 along with commercial β-glucosidase were added to the substrates, 17% and 14% of glucan was respectively converted for AFEX-treated corn stover and AFEX-treated rice straw. When the amount of E1-bearing rice extract was increased to 4 ml, 30% and 22% were respectively converted under the same conditions. No activity was observed when substrates were treated with non-transgenic (NT) rice total soluble protein.

Discussion

Plants have been used as “green bioreactors” for the production of essential enzymes (Hong, C. Y., Chen, K. J., Liu, L. F., Tseng, T. H., Wang, C. S. & Yu, S. M. Production of two highly active bacterial phytases with broad pH optima in germinating transgenic rice seeds. Transgenic Res. 13, 29-39 (2004); Chiang, C. M et al. Expression of a bifunctional and thermostable amylopullulanase in transgenic rice seeds leads to starch autohydrolysis and altered composition of starch. Mol. Breed. 15, 125-143 (2005)) and other proteins (Liu., H. L., Li, W. S., Lei, T., Zheng, J., Zhang, Z., Yan, X. F., Wang, Z. Z., Wang, Y. L. & Si, L. S. Expression of Human Papillomavirus type 16 L1 protein in transgenic tobacco plants. Acta Biochim Biophys Sin. 37, 153-158 (2005).), carbohydrates (Schulman, A. H. Transgenic plants as producers of modified starch and other carbohydrates. In: Plant biotechnology and transgenic plants. Edited by Kirsi-Marja Oksman-Caldenetey and Wolfgang H. Barz., N.Y., Basel., pp. 255-282 (2002); Sahrawy, M., Avila, C., Chueca, A., Canovas, F. M. & Lopez-Gorge, J. Increased sucrose level and altered nitrogen metabolism in Arabidopsis thaliana transgenic plants expressing antisense chloroplastic fructose-1,6-bisphosphatase. J. Exp. Bot. 55, 2495-2503 (2004)) and lipids (Qi, B. et al. Production of very long chain polyunsaturated omega-3 and omega-6 fatty acids in plants. Bio/technology 22, 739-745 (2004)) while requiring minimal inputs of raw materials and energy (Teymouri, F., Alizadeh, H., Laureano-Perez; L., Dale, B. E. & Sticklen, M. Effects of Ammonia Fiber Explosion Treatment on Activity of Endoglucanase from Acidothermus cellulolyticus in Transgenic Plant. Appl. Biochem. Biotechnol. 116, 1183-1192 (2004)). Production of biomolecules in plants, considered as molecular farming, is one approach to improve the economics and increase the low-cost production efficiency of these biomolecules (Bailey, M. R. et al. Improved recovery of active recombinant laccase from maize seed. Appl Microbiol Biotechnol. 63, 390-397 (2004); Breithaupt, H. GM plants for your health. EMBO. 5, 1031-1034 (2004); Fischer, R., Stoger, E., Schillberg, S., Christou, P. & Twyman, R. Plant-based production of biopharmaceuticals. Curr. Opin. Plant Biol. 7, 152-158 (2004)).

Several crops have been recommended for biomass-to-ethanol conversion, among them maize, rice, sugarcane and switchgrass (Knauf, M. & Moniruzzaman, M. Lignocellulosic biomass processing: A perspective. Internat Sugar Jour. 106, 147-150 (2004); Sticklen, M. et al. Production of microbial hydrolysis enzymes in biomass crops via genetic engineering. 2nd International Ukrainian Conference on biomass for energy, p. 133, 20-22 September 2004, Kyiv, Ukraine (2004))-all with a high amount of lignocellulosic biomass, and some of which have caused disposal problems. Production of enzymes in plants used for biomass conversion is a potentially powerful tool to facilitate the conversion of cellulose to glucose in the commercial production of ethanol while solving the problems associated with accumulated agricultural waste biomass. In addition, as ethanol bioconversion enzyme costs are decreased, ethanol biorefineries may achieve financial advantages over petroleum refineries.

In the present study, rice was genetically transformed with the catalytic domain of the E1 gene encoding the endo-1,4-β-glucanase (E1) enzyme. By immunoflouresence microscopic analysis, it was shown that the apoplast efficiently accumulated a high level of functional E1 enzyme, accounting for up to 4.9% of the plant total soluble protein (Table 10) with no apparent deleterious effects on plant growth, fertility and yield. Also, the E1 gene was stably inherited in a Mendelian manner (data not shown) and its E1 product remained active at high levels in the T1 generation.

There are three possible explanations why the heterologous E1 accumulated in apoplast did not harm transgenic plant cell walls. First, lignocellulose is difficult to hydrolyze because it is associated with hemicellulose, and surrounded by a lignin seal, which has a limited covalent association with hemicellulose. Moreover, it has a crystalline structure with a potential formation of hydrogen bonds resulting in a tightly packed structure. Also as per FIG. 26B, we conclude that pretreatment might be necessary to increase the surface area and consequently accessibility of cellulases by removing the lignin seal, solubilizing hemicellulose and disrupting crystallinity (Demain, A. L., Newcomb, M. & Wu, J. H. D. Cellulase, Clostridia, and Ethanol. Microbiol. Mol Biol Rev. 69, 124-154 (2005)). Second, cellulases function in a synergistic enzyme complex. If only one enzyme of the complex is expressed such as E1, this single enzyme might not be sufficient to significantly affect the integrity of the cell wall without the pretreatment (Ziegelhoffer, T. J., Raasch, A. & Austin-Phillips, S. Dramatic effects of truncation and subcellular targeting on the accumulation of recombinant microbial cellulase in tobacco. Mol. Breed. 8, 147-158 (2001)). Third, due to the thermophilic nature of the E1, the enzyme has limited activity at plant growth temperature (Dai, Z., Hooker, B. S., Anderson, D. B. & Thomas, S. R. Expression of Acidothermus cellulolyticus endoglucanase E1 in transgenic tobacco: biochemical characteristics and physiological effects. Trans. Res. 9, 43-54 (2000)).

When accumulated in cytosol, the normal level of heterologous protein production in plants is about 0.1-0.3% of plant total soluble proteins. In contrast when enzyme is targeted for accumulation in apoplast, this level has been increased to 4.9% in rice (Table 10) and up to 26% in Arabidopsis (Ziegler, M. T., Thomas, S. R. & Danna, K. J. Accumulation of a thermostable endo-1,4-b-D-glucanase in the apoplast of Arabidopsis thaliana leaves. Mol. Breed. 6, 37-46 (2000)). Among many factors (Cheng, M., Lowe, B. A., Spencer, T. M., Ye, X. & Armstrong, C. L. Factors influencing Agrobacterium-mediated transformation of monocotyledonous species. In Vitro Cell. Dev. Biol. 40, 31-45 (2004)), the use of the catalytic domain of the E1 gene (Ziegler, M. T., Thomas, S. R. & Danna, K. J. Accumulation of a thermostable endo-1,4-b-D-glucanase in the apoplast of Arabidopsis thaliana leaves. Mol. Breed. 6, 37-46 (2000).), the use of the Tobacco Mosaic Virus translational enhancer (Ibid.), the strength of the CaMV 35S promoter (Cheng et al. In Vitro Cell. Dev. Biol. 40, 31-45 (2004)) and the targeting of E1 enzyme to the apoplast (Ziegler et al.) might have contributed to the overall level of production of E1 in rice.

It has been well documented that cellulases work together synergistically to decrystallize and hydrolyze the cellulose. Exo-glucanases act on crystalline cellulose (on cellulose chain ends) and endo-glucanase (E1) acts on amorphous cellulose (interior portions of the cellulose chain) (Bayer, E. A., Chanzy, H., Lamed, R. & Shoham, Y. Cellulose, cellulases and cellulosomes. Curr. Opin. In Str. Biol. 8, 548-557 (1998)). In contrast, the results in this study demonstrate production of glucose at from atypical endoglucanase activity on a solid substrate. This could be due to two system features. First, the presence of hemicellulase (from β-glucosidase) in the reaction stream helped to remove the hemicellulose from the biomass, which in turn gave higher accessibility for the E1 enzyme to act more on the cellulose chains. Second, E1 enzyme can cause multiple random attacks on the same cellulose chain resulting in small fragments of cellobiose, cellotriose and cellotetraose. These fragments can be further hydrolyzed by enzyme molecules in solution such as E1 itself or β-glycosidase enzyme used to avoid reaction inhibition by cellobiose (Medve, J., Karlsson, J., Lee, D. & Tjerneld, F. Hydrolysis of microcrystalline cellulose by cellobiohydrolase I and endoglucanase II from Trichoderma reesei: Adsorption, sugar production pattern and synergism. Biotechnol. Bioeng. 59, 621-634 (1998)).

Based on a previous study, AFEX pretreatment, similar to other methods of pretreatments which operate under harsh conditions, might destroy as much as ⅔^{rd} of the activity of plant produced heterologous E1 (Teymouri, F., Alizadeh, H., Laureano-Perez; L., Dale, B. E. & Sticklen, M. Effects of Ammonia Fiber Explosion Treatment on Activity of Endoglucanase from Acidothermus cellulolyticus in Transgenic Plant. Appl. Biochem. Biotechnol. 116, 1183-1192 (2004)). Therefore, we recommend either producing higher level of E1 needed for hydrolysis, or extracting the E1 protein or the plant total soluble proteins concentrate containing the biologically active E1 enzyme and then adding the enzyme or the crude extract after pretreatment for the enzymatic hydrolysis of pretreated lignocellulosic matter, as we did in this study.

Production of E1 enzyme in plant biomass could potentially be commercially viable, with the caveat that for this potential to be fulfilled additional work is needed to produce other cellulase and possibly hemicellulase enzymes along with E1 in plants in order to maximize production of glucose and other sugars. When all are produced in plants, this could compete with the full range of commercial hydrolysis enzymes currently used in ethanol production. The costs of plant-produced enzyme conversion technology might further be reduced, and research ensuring that cellulases produced within the plants will survive harvest, storage, transportation and the thermo-chemical cellulosic material pretreatment step itself. The latter is a particularly formidable obstacle but may be achievable for pretreatments that use alkaline pH and more moderate temperatures than the dilute acid pretreatment, which operates at around 200° C. and pH 1.0.

This is the first report on production of a hydrolysis enzyme in rice. More experiments are needed to produce other hydrolysis enzymes in biomass crops, along with the use of bioconfinement methods to reduce seed contamination and controversies around genetically modified plants (NRC Report. Bioconfinement of genetically engineered organisms. The U.S. National Academy of Sciences. Natl. Acad. Sci. Press. 265 pages. (2004)).

Experimental Protocols

Transformation vector. The pZM766-E1cat containing the Acidothermus cellulolyticus E1 catalytic domain driven by the Cauliflower Mosaic Virus 35S Promoter (CaMV 35S), tobacco Mosaic Virus translational enhancer (Ω), and the tobacco pathogenesis-related protein 1a (Pr1a) signal peptide encoding sequence for apoplast-targeting of the E1 enzyme was removed from the pUC19 vector by digestion with XbaI. The removed cassette was transferred to the binary vector pCAMBIA 3301 containing the bar herbicide resistance selectable marker and the gus marker genes to generate the binary vector ApoE1.

Selection of transformants using glufosinate herbicide. A kill curve was developed to test the sensitivity of rice calli to glufosinate ammonium. Glufosinate ammonium was used as a selection agent since the binary vector used in this study contained the bar gene. Untransformed calli were placed on Murashige and Skoog (MS) and vitamins medium (Murashige, T. & Skoog, F. A revised medium for rapid growth and bioassays tobacco tissue cultures. Physiol. Plant 15, 473-497 (1962)) containing 0, 5.0, 10.0, 15.0, 20.0 and 25.0 mg/L of glufosinate ammonium for 6-8 weeks. The calli survival was recorded. All calli turned brown and died after being cultured on glufosinate ammonium with concentrations of 15 mg/L or more. Therefore, 15 mg/L glufosinate ammonium was used for transgenic plant selection.

Genetic transformation. Mature seeds of rice (Oryza sativa L. subsp. Japonica) variety Taipei 309 were dehusked and sterilized in 70% (vol/vol) ethanol for 2-3 min and then transferred into 50% (vol/vol) Clorox solution for 20 min with gentle shaking. The sterilized seeds were plated for callus induction on MS salts and vitamins medium supplemented with 3% sucrose, 300 mg/L casein enzymatic hydrolyzate, 500 mg/L proline, 2 mg/L 2,4-dichlorophenoxyacetic acid, 2.5 g/L Phytagel, pH 5.8 and grown for 21 days at 25° C. in the dark. The Agrobacterium strain LBA 4404 containing the transgenes was grown in 10 ml YM medium (containing Yeast extract 0.4 g/L, Mannitol 10.0 g/L, NaCl 0.1 g/L, MgSO4.7H2O 0.2 g/L, K₂HPO4.3H2O 0.5 g/L, pH 7.0) supplemented with 50 mg/L of kanamycin, streptomycin, rifampcin and 100 μM acetosyringone, incubated at 28° C. and 250 rpm for 48 h. Then, the cultures were transferred to 40 ml MS medium supplemented with 100 μM acetosyringone and incubated under the same conditions for another 24 h. The bacterial cells were harvested by centrifugation and resuspended in 15 ml of the same media. The cultures (cell density 0.9 at A₆₀₀) were used for transformation.

Three weeks after callus induction from the scutellar region of the rice embryo, embryogenic calli were immersed in A. tumefaciens suspension for 20 min under vacuum. Infected calli were co-cultivated in MS medium supplemented with MS salts and vitamins, 3% sucrose, 1% glucose, 500 mg/L casein enzymatic hydrolyzate, 2 g/L Gelrite, 100 μM acetosyringone, pH 5.2. After 3-4 days of co-cultivation, calli were washed with sterile water containing 500 mg/L cefotaxime and blotted on filter paper. The calli were immediately plated on a selection medium, calli induction medium supplemented with mg/liter glufosinate ammonium and 500 mg/L cefotaxime, pH 5.8, and incubated at 25° C. in the dark for 3-4 weeks. The calli that had proliferated after the initial selection were further sub-cultured for two selection cycles on the same medium every 2 weeks. The actively dividing glufosinate ammonium-resistant calli were plated on MS plant regeneration medium containing MS salts and vitamins, 3% sucrose, 3% sorbitol, 3 mg/L N6-benzyladenine, 1 mg/L naphthaleneacetic acid, 500 mg/L casein enzymatic hydrolyzate, 3 g/L Gelrite, 15 mg/L glufosinate ammonium, pH 5.8 and grown at 25° C. for a 10-h light/14-h dark photoperiod for 4 weeks. The regenerated plantlets were rooted on half-strength MS salts and vitamins, 4 g/L Gelrite, 15 mg/L glufosinate ammonium, pH 5.7. Plantlets were transferred to the greenhouse after acclimatization in growth chamber under 27° C. (day), 19° C. (night), 13 h photoperiod (13 h-light: 11 h-dark) and 210-300 mE light intensity.

Histochemical Analysis of GUS. Stable expression was assayed in plantlets from the transgenic lines and untransformed control via GUS histochemical staining using 5-bromo-4-chloro-3-indoyl-β-D-glucuronic acid salt α-gluc). Plantlets were immersed in the GUS substrate mixture and incubated at 37° C. (Jefferson, R. A., Kananagh, T. A. & Bevan, M. W. GUS fusions: β-glucuronidase as a sensitive and versatile gene fusion marker in higher plants. EMBO. 6, 3301-3306 (1987)), then incubated in 70% ethanol to remove the chlorophyll, and examined under a Zeiss SV8 stereomicroscope.

PCR analysis. PCR was used to detect bar and E1 genes in transgenic rice using leaf disk DNA as a template and REDExtract-N-Amp™ Plant PCR Kit (Sigma-Aldrich, St. Louis, Mo., Cat # XNA-P) based on the manufacturer's instruction. Primers for bar included the forward 5′-ATG AGC CCA GAA CGA CG-3′ (SEQ ID NO:29) and the reverse 5′-TCA GAT CTC GGT GAC GG-3′ (SEQ ID NO:30), and for the E1 included the forward 5′-GCG GGC GGC GGC TAT TG-3′ (SEQ ID NO:27) and the reverse 5′-GCC GAC AGG ATC GAA AAT CG-3′ (SEQ ID NO:28). DNA amplifications were performed in a thermo cycler (Perkin Elmer/Applied Biosystem, Foster City, Calif.) using initial denaturation at 94° C. for 4 min, followed by 35 cycles of 1 min at 94° C., 1 min at 55° C., 2 min at 72° C., and a final 10 minute extension at 72° C. The reaction mixture was loaded directly onto a 0.9% (w/v) agarose gel, stained with ethidium bromide and visualized with UV light. The transgene product size was about 0.59 kb for the bar gene and 1 kb for the E1 gene.

DNA isolation and Southern blot hybridization analysis. Confirmation of transgene integration into the plant genome, number of independent transgenic lines, and transgene copy numbers were performed by Southern blot hybridization using the E1-coding sequence as a probe. Genomic DNA from 5 randomly selected putative transgenic lines and untransformed rice plants was isolated using the protocol described in (Saghai-Maroof, M. A., Soliman, K. M., Jorgensen, R. A. & Allard, R. W. Ribosomal DNA spacer-length polymorphism in barley: Mendelian inheritance, chromosomal location, and population dynamics. Proc. Natl. Acad. Sci. USA. 81, 8014-8019 (1984); Ziegler, M. T., Thomas, S. R. & Danna, K. J. Accumulation of a thermostable endo-1,4-b-D-glucanase in the apoplast of Arabidopsis thaliana leaves. Mol. Breed. 6, 37-46 (2000)). For Southern blots, 8 μg of genomic DNA was digested with BstX1 restriction enzyme, electrophoresed in 1.0% (w/v) agarose gel, transferred onto Hybond-N+ (Amersham-Pharmacia Biotech) membranes, and fixed with a UV crosslinker (Stratalinker UV Crosslinker 1800, Stratagene, CA) as recommended in the manufacturers' instructions. The E1 gene-specific probe was generated using PCR amplification of the E1 gene to produce a 1.0-kb fragment. The amplified fragment was purified using the QIAquick kit (QIAGEN). Probe labeling and detection were obtained using the DIG High Prime DNA Labeling and Detection Starter Kit II (Kit for chemiluminescent detection with CSPD, Roche Co.), following the manufacturer's protocol.

RNA isolation and Northern blot hybridization analysis. Total RNA samples of the untransformed and transgenic plants were isolated from five different transgenic lines using the TRI Reagent (Sigma-Aldrich, St. Louis, Mo.) according to the manufacturer's instructions. Aliquots of RNA (20 μg) were fractionated in 1.2% agarose formaldehyde denaturing gel and blotted on a Hybond-N+ nylon membrane (Amersham Pharmatica Biotech) as specified by the manufacturer. The E1 gene-specific probe was generated using PCR amplification of the E1 gene to produce a 1.0-kb fragment. The fragment was gel purified using the QIAquick Gel Extraction Kit (QIAGEN Inc., Valencia, Calif.). Probe labeling and transcript detection were obtained using the DIGHigh Prime DNA Labeling and Detection Starter Kit II (Kit for chemiluminescent detection with CSPD, Roche Co.), following the manufacturer's protocol.

Protein Extraction and Western Blot Analysis.

Electrophoresis and transfer. Plant total soluble protein was extracted from a reported protocol (Ziegler, M. T., Thomas, S. R. & Danna, K. J. Accumulation of a thermostable endo-1,4-b-D-glucanase in the apoplast of Arabidopsis thaliana leaves. Mol. Breed. 6, 37-46 (2000)) using the Invitrogen NuPAGE® Bis-Tris Discontinuous Buffer System with the 10% NuPAGE® Novex Bis-Tris Pre-Cast Gel. Total soluble protein (1 μg), NuPAGE® LDS Sample Buffer (5 μl), NuPAGE® Reducing Agent (2 μl), and deionized water were mixed to a total volume of 20 μl. The samples were heated at 70° C. for 10 minutes prior to electrophoresis using the XCell SureLock™ Mini-Cell with NuPage® MES SDS Running Buffer. The gel was run for about 45 minutes at 200 V, and blotted onto a membrane using the XCell II® Blot Module and NuPAGE® Transfer Buffer at 30 V for 1 hour, following the manufacturer's protocol.

Blocking, incubation and detection. The membrane was placed into blocking buffer (1×PBS, 5% non-fat dry milk, 0.1% Tween 20) immediately after transfer and incubated at room temperature for 1 hour with gentle agitation. The primary antibody (mouse anti-E1, provided by Steven Thomas, National Renewable Energy Laboratories) was diluted in blocking buffer to a concentration of 1 μg/ml. The blocking buffer was decanted from the membrane, ml of antibody solution was added, and the membrane was incubated at room temperature for 1 hour with gentle agitation. The primary antibody solution was decanted and the membrane was washed in washing buffer (1×PBS, 0.1% Tween 20) for 30 minutes with gentle agitation at room temperature, changing the wash solution every 5 minutes. The enzyme conjugate anti-mouse IgG:HRPO (Transduction Laboratories) was diluted 1:2000 in blocking solution and added to the membrane after decanting the wash buffer. The membrane was incubated with the secondary antibody solution for 1 hour at room temperature with gentle agitation; the antibody solution was decanted from the membrane and the membrane was washed in washing solution as before. For detection, 1 ml each of Stable Peroxide Solution and Luminol/Enhancer Solution (Pierce SuperSignal® West Pico Chemiluminescent Substrate) were mixed and incubated with the membrane for 5 minutes. The membrane was blotted slightly to remove excess substrate and placed in a plastic envelope. Excess liquid and air bubbles were removed. The blot was exposed to X-ray film (Kodak BioMax XAR Scientific Imaging Film) and developed in a Kodak RP X-OMAT Processor.

Immunofluorescence Microscopic Analysis.

Tissue Preparation and Immunofluorescence labeling: Free-hand sections of fresh leaf tissue from transgenic and untransformed rice plants were isolated and hydrated in NaCl/Pi buffer (0.8% NaCl, 0.02% KCl, 0.14% Na₂HPO₄.2H₂O, and 0.02% KH₂PO₄ in water) containing 0.5% BSA (BSA/NaCl/Pi) for 2 min. Sections were incubated in primary antibody (rabbit anti-(mouse IgG)) raised against the E1 enzyme diluted 1:250 in the same buffer, in a moist chamber for 3 hours. The primary antibody was rinsed off with the BSA/NaCl/Pi buffer and sections were incubated for 2 hours at room temperature with fluorescein isothiocyanate (FITC)-conjugated secondary antibody (goat anti-(rabbit whole molecule IgG)) diluted 1:250 in the same buffer using same moist chamber. The secondary antibody was then rinsed off with the same buffer.

Fluorescence microscopy: Intracellular localization of the FITC-labeled protein was observed and images were taken using a confocal laser scanning microscopy Zeiss LSM 5 Pascal (Carl Zeiss, Jena, Germany) FITC fluorescence and chloroplast autofluorescence was excited with an argon ion laser, λₑₓ=488 nm. Fluorescence emission was detected through a Band Pass (BP) filter, λₑₘ=530/30 nm for the FITC (images represented in green) and Long Pass (LP) filter, λₑₘ=650 nm for the chloroplast (images represented in red). Either a 63X Plan-apochromat or a 20X Plan-neofluar objective lens was used.

The Biological Activity Assays of Heterologous E1 Enzyme.

MUCase. After seedlings developed reasonable leaf size, the MUCase enzyme assay was conducted as reported (Ziegler, M. T., Thomas, S. R. & Danna, K. J. Accumulation of a thermostable endo-1,4-b-D-glucanase in the apoplast of Arabidopsis thaliana leaves. Mol. Breed. 6, 37-46 (2000); Ziegelhoffer, T. J., Raasch, A. & Austin-Phillips, S. Dramatic effects of truncation and subcellular targeting on the accumulation of recombinant microbial cellulase in tobacco. Mol. Breed. 8, 147-158 (2001)). E1 enzyme activity was determined by subtracting the background contributed by Taipei 309 rice control extracts, from the spectrophotometer fluorescence readings. The resulting fluorescence signals without noise were used to calculate the activity and amount of biologically active E1 enzyme present in transgenic samples.

CMCase. 24-well agar plates containing 1% carboxymethylcellulose (CMC) were exposed to leaf total soluble protein extract. The plates were heated to 65° C. for 30 min in an oven to activate the enzyme. The plates were cooled at 4° C. for 5 min and then stained with 1 mg/ml Congo Red for 30 min as described (Wood, P. J., Erfle, J. D. & Teather, R. M. Use of complex formation between Congo Red and polysaccharides in detection and assay of polysaccharide hydrolases. Meth. Enzymol. 160, 59-75 (1988)). Samples were destained with 1 M NaCl for 5 min and fixed with 10 mM NaOH.

Cellulose Hydrolysis Assay.

Pretreatment of Biomass. Milled corn stover and rice straw (about 1 cm in length) were pretreated using Ammonia Fiber Explosion technique (AFEX). The biomass was transferred to a high pressure Parr reactor with 60% moisture (kg water/kg dry biomass) and liquid ammonia ratio 1.0 (kg of ammonia/kg of dry biomass) was added. As the temperature was slowly raised, the pressure in the vessel increased. The temperature was maintained at 90° C. for five minutes before explosively releasing the pressure. The instantaneous drop of pressure in the vessel caused the ammonia to vaporize, causing an explosive decompression and considerable fiber disruption. The pretreated material was kept under a hood to remove residual ammonia and stored in a freezer until further use.

Substrate hydrolysis: E1 activity was measured by reacting total protein extracted from E1-expressed rice leaves with different substrates, namely: AFEX-treated corn stover (CS), AFEX-treated rice straw (RS), CMC and Avicel. Commercial cellulase enzyme (Spezyme CP, Genencor International) was used in this experiment as a control. The enzyme hydrolysis was done in a sealed scintillation vial. A reaction medium composed of 7.5 ml of 0.1 M, pH 4.8 sodium citrate buffer was added to each vial. In addition, 60 μl (600 μg) tetracycline and 45 μl (450 μg) cycloheximide were added to prevent the growth of microorganisms during the hydrolysis reaction. The reaction was supplemented with 30 CBU of β-glycosidase enzyme (Novo 188 from Sigma) to avoid inhibition by cellobiose. Distilled water was then added to bring the total volume in each vial to 15 ml. All the reactions were done in duplicate to test reproducibility. All hydrolysis reactions were carried out at 50° C. with a shaker speed 90 rpm. About 1 ml of sample was collected at 168 hours of hydrolysis, filtered using a 0.2 μm syringe filter and kept frozen. The amount of glucose produced in the enzyme blank and substrate blank were subtracted from the respective hydrolyzed glucose levels.

Sugar analysis: Hydrolyzate was quantified using Waters HPLC by running the sample in Aminex HPX-87P (Biorad) column, against sugar standards. The amount of glucose produced in the enzyme blank and substrate blank were subtracted from the respective hydrolyzate glucose levels.

TABLE 10
The amount of heterologous E1 enzyme in different independent
transgenic rice events determined by the MUCase activity
assay (average of 3 reps).

| | Posi- | Neg- | | | | | |
|---|---|---|---|---|---|---|---|
| | tive | a- | | | | | |
| | con- | tive | | | | | |
| | trol | control | Line 1 | Line 2 | Line 3 | Line 4 | Line 5 |
| E1 in | 3.60% | 0 | 3.87% | 2.67% | 2.41% | 4.90% | 2.85% |
| the | | | | | | | |
| total | | | | | | | |
| solu- | | | | | | | |
| ble | | | | | | | |
| pro- | | | | | | | |
| tein | | | | | | | |

### EXAMPLE 13

This Example relates to the expression of biologically active Acidothermus cellulolyticus endoglucanase in transgenic maize plants.

Commercial production of ethanol from plant biomass sources employs enzymatic hydrolysis of cellulose to glucose. Transgenic plants that can produce their own hydrolysis enzymes offer an inexpensive and convenient system for the large-scale production of these enzymes. The catalytic domain of a endo-1,4-β-D-glucanase gene from the eubacterium, Acidothermus cellulolyticus, was transferred to the maize using particle bombardment, and 31 independent transgenic plants were regenerated from five independent experiments containing E1 catalytic domain. Several of these plants grown in the greenhouse reached maturity and a few of them set seeds. Stable integration of the transgene in the genome of these plants was confirmed by Southern blot analysis and expression of the transgene in plants by Western blot analysis. Expression of the recombinant E1-cd varied in independent transgenic plants and the protein was enzymatically active at elevated temperatures. The activity-based assays indicate that the enzyme accumulated to concentrations up to 2.1% and 2.08% of the total soluble protein in leaf and root tissues, respectively. The present data demonostrate the feasibility to produce bacterial cellulase in a widely grown biomass crop plant for biomass conversion.

Abbreviations: E1-cd, catalytic domain of Acidothermus cellulolyticus endo-1,4-β-glucanase E1; MUC, 4-methylumbelliferyl-β-D-cellobiose; PCR, polymerase chain reaction; Pr1a, tobacco pathogenesis-related protein 1a; MES, 2-(N-morpholino)ethanesulfonic acid.

Maize (Zea mays L.) is one of the largest grown annual crops cultivated worldwide. In the USA alone, maize production reached nearly 300 million metric tons in 2003 (World wheat, corn, and rice production. http://nue.okstate.edu/Crop_Information/World_wheat_production.htm). Large over production of this subsidized crop decreases its market value and forces producers to find new uses of their commodity. Genetic engineering has the potential to improve the economic value of maize by introducing genes to improve its adaptability and agronomic characteristics, and to increase its utilization in non-traditional areas such as production of industrial raw materials, enzymes and other useful compounds (J. K-C. Ma, P. M. W. Drake and P. Christou, The production of recombinant pharmaceutical proteins in plants, Nature genetics. 4 (2003), pp. 794-805). One attractive approach is to engineer maize to produce polysacaride-degrading enzymes such as cellulases to serve as a “green bioreactor”. Because of its high biomass production, it has been identified as ideal candidate for biomass fuel production. Approximately, 45% of dry mass of plant biomass comprised of cellulosic materials, of which cellulose is the largest single fraction biopolymer, constitute of 30-50% (C. E. Wyman. Production of low cost sugars from biomass: progress, opportunities, and challenges: In: R. P. Overend, E. Cornet, Editors, Biomass—a growth opportunity in green energy and value added products, Proceedings of the 4th Biomass conference of the Americas, pergamon, oxford, 1 (1999), pp. 867-872). Enzymatic conversion of cellulose to metabolizable sugars is a crucial step for further conversion to other useful products, including ethanol production. The conversion of cellulosic biomass into useful products is a complex process and involves synergistic action of three different enzymes, such as endo-1,4-β-glucanase (EC.3.2.1.4), exo-cellobiohydrolase (EC.3.2.1.91), and β-glucosidase (EC.3.2.21).

Genes for a variety of cellulase enzymes from fungi and bacteria have been cloned and characterized (S. Shoemaker, V. Schweickart, M. Lander, D. Gelfand, S. Kwok, K. Myambo and M. Innis, Molecular cloning of exo-cellobiohydrolase I derived from trichoderma reesei strain L27, Biotechnology 1 (1983), pp. 691-696.) and (J. O. Kim, S. R. Park, W. J. Lim, S. K. Ryu, M. K. Kim, C. L. An, S. J. Choo, Y. W. Park, J. H. Kim and H. D. Yun, Cloning and characterization of thermostable endoglucanase (Cel8Y) from the hyperthermophilic Aquifex aeolicus VF5, Biochemical and Biophysical Research Communication 279 (2000), pp. 420-426). Production of cellulase in microbial systems has been studied extensively (A. L. Demain, M. Newcomb and J. H. D. Wu, Cellulase, clostridia and ethanol, Microbiology and Molecular biology Reviews 69 (1) (2005), pp. 124-154), but studies involved its production in crop plants is very limited (A. M. Nuutila, A. Ritala, R. W. Skadsen, L. Mannonen and V. Kauppinen, Expression of fungal thermotolerant endo-1,4-β-glucanase in transgenic barley seeds during germination. Plant Molecular Biology 41 (1999), pp. 777-783). To date, cellulase enzymes are produced from microorganisms, however, the production costs of commercial cellulase enzyme preparation from these sources are very high and prohibit large-scale bioconversion from cellulosic biomass to ethanol.

Transgenic plants that overexpress different cellulases offer an alternative as “green bioreactors” to produce inexpensive and sufficient amounts of cellulases with almost limitless potential for scale-up, which has been a major goal for other important industrial enzymes and pharmaceutical proteins.

Earlier work has demonostrated the feasibility of the production of cellulase in dicotyledonous plants namely, Arabidopsis (M. T. Ziegler, S. R. Thomas and K. J. Danna, Accumulation of a thermostable endo-1,4-β-D-glucanase in the apoplast of Arabidopsis thaliana leaves. Molecular Breeding 6 (2000), pp. 37-46), tobacco (T. Ziegelhoffer, J. A. Raasch and S. Austin-phillips, Dramatic effects of truncation and sub-cellular targeting on the accumulation of recombinant microbial cellulase in tobacco. Molecular Breeding 8 (2001), pp. 147-158.) and potato (Z. Dai, B. S. Hooker, D. B. Anderson and S. R. Thomas, Improved plant-based production of E1 endoglucanase using potato: expression optimization and tissue targeting. Molecular breeding 6 (2000), pp. 277-285). In this study, we have examined the expression of the catalytic domain of a thermostable endo-1,4-β-D-glucanase E1 cloned from Acidothermus cellulolyticus, in transgenic maize plants.

Materials and Methods

Preparation of embryogenic callus: immature zygotic embryos (approximately 1.5-2.0 mm long) of Hi II maize germplasm (C. L. Armstrong, C. E. Green and R. L. Phillips, Development and availability of germplasm with high Type II culture formation response. Maize Genetics Cooperative Newsletter 65 (1991), pp. 92-93), were cultured embryo-axis-side down on N6-based media (C. C. Chu, Wang, C. S. Sun, C. Hsu, K. C. Yin, C. Y. Chu and F. Y. Bi, Establishment of an efficient medium for anther culture of rice through comparative experiments on the nitrogen source, Sci. Sin. 18 (1975), pp. 659-668) supplemented with 50 μM silver nitrate, 100 mg L⁻¹ casein hydrolysate, 25 mM L-proline (C. L. Armstrong and C. E. Green, Establishment and maintenance of friable, embryogenic maize callus and the involvement of L-proline, Planta 164 (1985), pp. 207-214), 2 mg L⁻¹ 2,4-dichlorophenoxy acetic acid, 30 g L⁻¹ sucrose and 2.5 g L⁻¹ Gelrite (Sigma chemical Co., St. Louis, Mo.) at pH 5.8 (referred to as N6E). Culture plates were wrapped with parafilm and incubated for 2 weeks at 28° C. in the dark. After 2 weeks, calluses produced from the scutellum were selected and subcultured onto fresh N6E medium. As the number of embryogenic calluses increased, they were distributed to new plates of N6E medium and subcultured every two weeks until required.

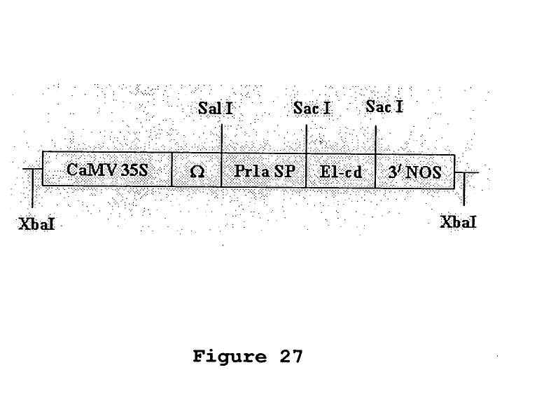

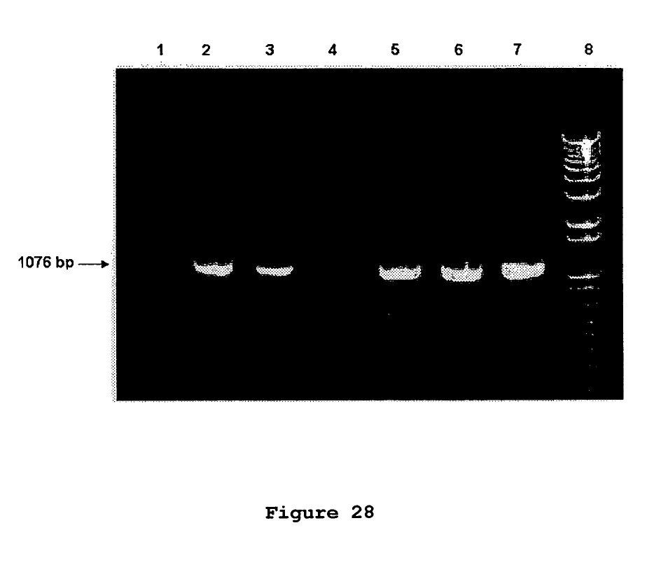

Transforming plasmids: The pMZ766 (M. R. L. Owen, J. Pen, Editors, Transgenic plants: A production system for industrial and pharmaceutical proteins. John wiley, London (1996)) containing the catalytic domain of a endo-1,4-β-D-glucanase gene isolated from the eubacterium, Acidothermus cellulolyticus (E1) was fused to the sequence encoding the tobacco Pr1a signal peptide. The fragment containing the signal peptide and E1 catalytic domain was fused in frame down stream of the Cauliflower mosaic 35S promoter, carrying tobacco mosaic virus Ω translational enhancer, and upstream of the polyadenylation signal of nopaline synthase (FIG. 27) This plasmid was mixed in a 1:1 ratio with the plasmids pDM302 (J. Cao, X. Duan, D. McElroy and R. Wu, Regeneration of herbicide resistant transgenic rice plants following microprojectile-mediated transformation of suspension culture cells, Plant Cell Rep. 11 (1992) pp. 586-591) or pBY520 (D. Xu, X. Duan, B. Wang, B. Hong, T. D. Ho and R. Wu, Expression of a late embryogenesis abundant protein gene, hva1, from barley confers tolerance to water deficit and salt stress in transgenic rice, Plant Physiol. 110 (1996), pp. 249-257) containing the bar selectable marker gene.

Microprojectile bombardment: Tungsten particles of an average size of 0.7 μm (M10, Bio-Rad) were washed and coated with the plasmid DNA. The coated particles were bombarded according to the procedures described in the BioRad PDS 1000/He® biolistic gun instruction manual. Briefly, prewashed 50 μl aliquots of tungsten particles (50 mg/ml of glycerol) were mixed with 10 μl of plasmid DNA (1.0 μg/μl), 50 μl CaCl₂ (2.5 M) and 20 μl spermidine (0.1 M) in a microfuge tube by vortexing after each addition. The mixture was centrifuged for 20 sec and the supernatant removed. The DNA-coated particles were washed in 250 μl ethanol and resuspended in 50 μl of ethanol. Five microliters of the DNA-coated particles was pipetted onto each macrocarriers while the suspension was continuously vortex. Prepared macrocarriers were placed in the laminar hood to maintain maximum dryness prior to bombardment. For bombardment, rapidly growing callus pieces 3-5 days after subculture were evenly distributed within a 1.5 cm diameter target area in the center of a plate on osmotic medium 2 h prior to bombardment. Osmotic medium was identical to the callus initiation medium but contained 0.2 M sorbitol and 0.2 M mannitol (P. Vain, M. D. McMullen and J. J. Finer, Osmotic treatment enhances particle bombardment-mediated transient and stable transformation of maize, Plant Cell Rep. 12 (1993), pp. 84-88). Each plate was bombarded once using the Biolistic particle acceleration device (PDS 1000, Bio-Rad) with a rupture disk pressure of 1100 psi; 6.5 cm target distance (from middle of launch assembly to target plate) under a chamber pressure of 27 mm Hg. After bombardment, callus pieces were kept in respective plate for another 15 h and then transferred off the osmotic medium to callus inition medium. Culture plates were wrapped with parafilm and maintained at 28° C. in the dark for 5 days before being placed under selection pressure.

Selection of stable transformants: The selection medium was similar to that used for callus initiation medium but without proline and casein hydrolysate, and with 2 mg L⁻¹ bialaphos. Callus pieces were transferred every 2 weeks to fresh selection media. After 3-4 cycles of selection, white, rapidly growing callus clusters were picked out from non proliferating and partially necrotic mother calli, and transferred onto fresh selection medium. They were subcultured every two weeks and maintained in the dark at 28° C. during which embryogenic pieces were selected for plant regeneration.

Plant regeneration, acclimatization and transgenic seed recovery: Small pieces of resistant callus with clearly defined somatic embryos, were transferred to MS media (T. Murashige and F. Skoog, A revised medium for rapid growth and bioassays with tobacco tissue culture, Physiol. Plant. 15 (1962), pp. 473-497) containing 6 g L⁻¹ maltose, 1 mg L⁻¹ bialaphos, and 3 g L⁻¹ Gelrite. Five to six callus pieces were transferred to 100×25 mm plastic petri plates containing 25 ml of medium. The plates were maintained under fluorescent light (60 μmol quanta m−²·s−¹ from cool-white 40 W Econ-o-watt fluorescent lamp; Philips Westinghouse, USA) with a 16/8-h photoperiod at 25° C. Emerging plantlets with fully formed small shoots and roots were transferred onto 30 ml of rooting medium containing half-strength MS salts and vitamins, 15 g L⁻¹ sucrose and 2.0 g L⁻¹ gelrite without growth regulators in Magenta GA7 vessels (65 mm. 65 mm. 100 mm; Magenta corp., Chicago, USA). Rooted plantlets were thoroughly washed with tap water from the medium and transferred to small pots (4 inch²) containing pre-wetted soil (BACTO high porosity professional planting mix; Houston, Tex.). Plants were acclimated from the highly humid culture condition to the greenhouse environment under plastic bags, where small holes were made in the bag every other day for two weeks. The acclimated plants were transplanted to 2-gallon pots containing soil and grown under greenhouse condition. Plants were watered daily and fertilized with peter (20-20-20) granular fertilizer (Scotts company, Marysville, Ohio) weekly until they reached maturity. Transgenic plants were self-pollinated or cross-pollinated with plants originating from the same transformation events. In some cases, transgenic ears were pollinated with wild type pollen due to lack of transgenic pollen. Seed was dried down on the plant and harvested 35-45 days after pollination.

PCR and Southern blot analysis: Total genomic DNA was isolated from the leaves of greenhouse-grown independent plants. The leaves were exposed to liquid N₂ and ground to a fine powder. DNA was extracted with a modified CTAB-protocol (H. G. Murray and W. F. Thompson, Rapid isolation of high molecular weight plant DNA, Nucleic Acids Res. 8 (1980), pp. 4321-4326) and quantified after RNAse treatment. Plants were screened using PCR amplification for the introduced endo-1,4-β-D-glucanase gene. The oligonucleotide primers, 5′-GCGGGCGGCGGCTATTG-3′ (SEQ ID NO:31) and 5′-GCCGACAGGATCGAAAATCG-3′ (SEQ ID NO:32), were used to amplify a 1.0 kb fragment spanning the catalytic domain of the endo-1,4-β-D-glucanase gene which was analysed by electrophoresis in 0.8% agarose/ethidium bromide gels. For Southern analysis, genomic DNA (15 μg per lane) was loaded onto gels with or without digestion with Sac I endonuclase. Following electrophoresis in an 0.8% agarose gel, DNA was transferred to a Hybond-N membrane (Amersham-Pharmacia Biotech; Buckinghamshire, UK), fixed to the membrane by UV-crosslinking. The catalytic domain of the endo-1,4-β-D-glucanase gene was labeled with digoxigenin-11-dUTP and used for probing the domain. Hybridization and chemiluminescence signal detection were performed according to the manufacturer's instructions. Transgene copy numbers were estimated by including on the Southern blot, plasmid DNA equivalent to 2, 4, 10, and 20 transgene copies added to non-transformed maize DNA.

Western blot analysis: Protein sample were obtained from approximately 100 mg of leaf material from the non-transgenic and transgenic lines, by grinding the tissue to a fine powder in liquid N₂. Subsequent homogenization in 200 ul plant protein extraction buffer (50 mM sodium acetate PH 5.5, 100 mM Nacl, 10% v/v glycerol, 0.5 mM EDTA, 1 mM phenylmethylsulfonyl fluoride and 1 mg/l each of aprotinin, leupeptin and pepstatin) (T. Ziegelhoffer, J. A. Raasch and S. Austin-phillips, Dramatic effects of truncation and sub-cellular targeting on the accumulation of recombinant microbial cellulase in tobacco. Molecular Breeding 8 (2001), pp. 147-158) was performed, followed by a centrifugation at 15000×g for 5 minutes to remove the precipitate. Total protein concentration in each sample was measured by the Bradford method (M. Bradford, A rapid and sensitive method for quantitation of microgram quantities of protein utilizing the principle of protein-dye binding. Anal Biochem. 72 (1976), pp. 248-254) using Bio-rad protein assay reagent (Bio-Rad; CA). Approximately, 1 μg of total soluble protein from each samples were heated at 70° C. for 10 min and loaded onto 10% NuPAGE® Novex Bis-Tris Pre-Cast Gels (Invitrogen; Carlsbad, Calif.) and separated by electrophoresis, using the Xcell surelock Mini-cell with Nupage MES SDS running buffer (Invitrogen; Carlsbad, Calif.). The separated proteins were then transferred to nitrocellulose membrane (Hybond™ ECL™; Amersham-Pharmacia Biotech, Buckinghamshire, UK). The membrane was blocked for 1 hour with blocking buffer (1×PBS, 5% non-fat dry milk, 0.1% Tween 20). The membrane was probed with primary antibody (mouse anti-E1 monoclonal antibody), then rinsed with washing buffer (1×PBS, 0.1% Tween 20), and probed with secondary antibody (anti-mouse IgG: HRPO, Transduction Laboratories) for another hour. Finally, the membrane was washed in washing buffer for 30 minutes, followed by incubation in SuperSignal® west pico (Pierce Biotechnology Inc; Rockford, Ill.) chemiluminescent substrate for the horseradish peroxidase reaction and developed in a Kodak RP X-OMAT processor.

MUCase assay: The 4-methylumbelliferyl-β-D-cellobiose (MUC) assay was bassed on the ability of endoglucanase E1 to hydrolyze the fluorogenic substrate 4-methylumbelliferyl-β-D-cellobioside to produce the fluorophore, 4-methylumbelliferone (MU). Approximately 100 mg leaf or root tissues from each sample was pulverized in liquid N₂ and total soluble proteins was extracted from the samples as described by (Ziegelhoffer et al., ibid, (2001)), but without cellulysin and macerase pretreatment. The total amount of protein in each of the extracts was measured by the Bradford method (Bradford, M., Anal. Biochem. 72: 248-254 (1976)) using the Bio-Rad protein assay reagent (Bio-Rad; CA). Subsequently the appropriate amount of extract containing the same amount of total protein (10 ng or 100 ng) was subjected to the activity assay as described (Ziegelhoffer, ibid, (2001)). The reaction was stopped by adding 100 μl of 150 mM glycine pH 10 and the relative amount of released MU was measured with a Spectramax M2 (Molecular Devices Corporation, Sunnyvale, Calif.) at excitation and emission wavelengths of 360 nm and 465 nm, respectively. The activity of the transgenic plants was calculated after subtracting the background activity contributed by the wild type plants.

Results.

Recovery of transgenic maize plants: After 8 weeks of selection, the transformed calli became morphologically distinguishable from nontransformed calli. While control non-transformed calli died in selection medium, the bialaphos resistant calli continued to proliferate in presence of selection pressure. Bialaphos-resistant calli transferred to the maltose-containing regeneration medium readily regenerated to plantlets after 3-4 weeks. Over 2000 plantlets were regenerated from a total of 42 independent bialaphos-resistant calli (Table 11). Fourty two of these plantlets were grown to maturity in the greenhouse, where some set seeds after self or cross pollination. Of the 42 plants, 31 confirmed the presence of 1.0 kb E1-cd using PCR amplification. A representative sample of five lines transformed with the E1-cd cassette are shown in FIG. 28.

TABLE 11
Transformation frequencies and regeneration of transgenic
plants based on fresh weight of maize type II callus.

| | Total FW | Number of | |
|---|---|---|---|
| | (g) of | independent | Plants |
| | callus | bialaphos | containing |
| | pieces | reistant callus | E1-cd |
| Experiments | bombarded | clones | cassatte |
| 1 | 4 | 7 | 5 |
| 2 | 8 | 10 | 7 |
| 3 | 4 | 8 | 6 |
| 4 | 6.4 | 9 | 6 |
| 5 | 4 | 8 | 7 |

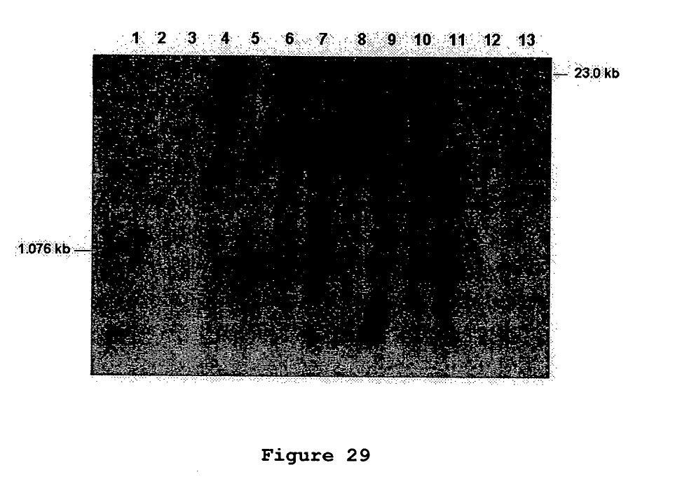

Southern blot analysis using undigested and digested total genomic DNA isolated from the leaves of PCR positive E1-cd plants confirmed the stable integration of E1-cd in the genome of these plants. After digestion of genomic DNA with Sac I, all of the five PCR positive plants showed the expected 1.0 kb diagnostic fragment (FIG. 29), corresponding to the E1-cd construct used. A separate Southern blot (data not shown) showed that 2 to 20 copies of the E1-cd transgene were integrated into the maize genome. With undigested genomic DNA, only fragments larger than 23.0 kb hybridized, indicating that the transferred gene had integrated into the genome of all plants analysed. No hybridization signal was present in any of the lanes containing DNA, digested or undigested from the control, untransformed plants.

Inheritance of the E1-cd transgene in 21 progenies of self-crossed plants was determined by PCR analysis and 3:1 segregation was found (data not shown), indicating that transgene was stably inherited and inserted in a single locus on one chromosome.

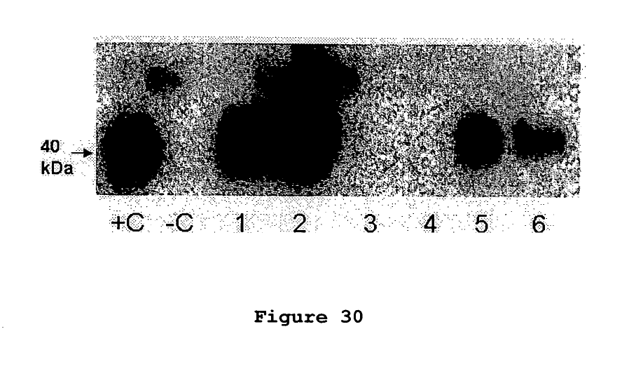

Expression of E1-cd in transgenic maize plants. Production of E1-cd protein in the regenerated plants was examined immunologically by using a monoclonal antibody against E1-cd. Protein extracts were prepared from the individual primary plants, representing six independent events and separated by electrophoresis on a SDS-PAGE gel. A band of approximately 40 KDa was detected in 4 events transformed with the chimeric E1-cd cassatte (FIG. 30). The band was similar in size to authentic immunoreactive band of E1-cd of transgenic tobacco plants, which was included as a positive control. In contrast, a control sample from non-transformed maize plants exhibited no immunoreactive band.

Production of biologically active E1-cd in transgenic maize plants: Different transgenic plants showed different levels of E1 production. The accumulation of E1 in most of the plants was less than 2% of total plant soluble proteins. A broad range of endoglucanase activity was observed among 50 tested transformants (25 independent transgenic plants), of which 63% of the transgenic plants showed an extremely low level (0.000002 nMol/μg soluble protein/min) of activity and 27% showed moderate level (i.e 0.0087 nMol/μg soluble protein/min) of activity (not shown in the table). The remaining 10% plants showed compairably high level (i.e 0.429 nMol/ug soluble protein/min) of activity (Table 12). The highest levels of activity achieved were observed in extracts from plant M4, with 0.845 nMol/min/μg protein in leaf and 0.835 nMol/min/μg protein in root tissue. In this plant, the recombinant E1-cd accumulated for ca. 2.1% of total soluble protein in leaf extracts and ca. 2.08% of total soluble protein in root extracts.

TABLE 12
E1-cd specific activity in protein extracts of leaf and root tissues
of selected individual transgenic maize plants expressed
as nMol MU per microgram total soluble protein per min.

nMol/min/
microgram
protein

| Transgenic Event | Leaf | Root |
|---|---|---|
| M1 | 0.432 | 0.537 |
| M2 | 0.330 | 0.029 |
| M3 | 0.360 | 0.131 |
| M4 | 0.845 | 0.835 |
| M5 | 0.178 | 0.017 |

The data presented in the table are the average values of three independent assays.

Microprojectile method of DNA transfer used in the present study has been used routinely to transform maize plants (Gordon-Kamm et al., Plant Cell 2, 603-618 (1990), Zhong et al., ibid, (1996) and Frame et al., In Vitro Cell Dev. Biol. Plant, 36: 21-29 (2000)). Stably transformed callus pieces were routinely recovered after 6-8 weeks of selection on bialaphos containing medium. Bialaphos has been used widely as a selective substrate for maize transformation (Dennehey et al., Plant Cell Tiss. Org. Cult., 36: 1-7 (1994) and Ishida et al, Nature Biotechnol., 14: 745-750 (1996)). It is well-documented that maltose enhances plant regeneration frequency in rice (Ghosh, et al., J. Plant Physiol., 139: 523-527 (1993)) and, consequently, it is used in maize plant regeneration media. Interestingly, all the transformed callus pieces readily regenerated to plants upon transferred to the maltose-containing medium. The beneficial effect of maltose as a sole carbohydrate source on maize plant regeneration is likely attributable to better bioavailability of the maltose during embryo germination.

PCR and Southern analysis confirmed that 31 out of 42 transgenic plants carried the catalytic domain of E1 gene and in most cases, multiple copies of the transgene were integrated into the genome. Multiple copies of transgene integration pattern is common in transgenic maize plants produced by particle bombardment (Shou et al., Mol. Breeding, 13: 201-208 (2004) and Kennedy et al, Plant Cell Rep. 20, pp. 721-730 (2001). Functional transformation is associated with the normalized expression of the transgene, especially in translational level. Therefore, an E1-cd-specific monoclonal antibody was used to characterize the E1-cd expression in protein extracted from six independent transgenic plants. It was found that 4 out of 6 transgenic plants displayed E1-cd expression, although the accumulation of polypeptide varied substantially in independent transgenic plants. For example, transgenic plants 1 and 2, exhibited high expression and transgenic plants 5 and 6, exhibited relatively low expression. Conversely, transgenic plants 3 and 4, failed to express the transgene, presumably, due to transgene silencing. In nuclear transformation, transgene expression heterogenity could reflect the influence of many factors, including position effects, transgene structure, integrative fragmentation, transgene copy numbers, rearrangement and epigenetic phenomena such as homology-dependent transcriptional silencing and cosuppression (Allen, ibid, (1993), Rathore et al., Plant Mol. Biol. 21, pp. 871-884 (1993) and Hart, et al., Mol. Gen. Genet. 235, pp. 179-188 (1992)), and the presence of boundary elements or MARs. Transgene integrated at subtelomeric regions may be strongly expressed (Topping et al, Development, 112: pp. 1009-1019 (1991). Conversely, areas rich in heterochromatin, such as those surrounding centromeres, may exert strongly negative position effects, and transgenes integrated at such sites may be prone to silencing (Prols et al., Plant J. 2: pp. 465-475 (1992) and Rathore et al, ibid, (1993)).

Since the catalytic domain of E1 is from a thermophilic bacterium, the enzyme is most active at elevated temperatures (U.S. Pat. No. 5,275,944 to Himmel et al). Therefore, the activity of the translational product was monitored with MUCase assay at 65° C. When we analyzed the E1 activity in the leaf and root protein extracts, the overall average activity was found to be slightly higher in the leaf protein extracts than in root protein extracts. Different tissues within plant are expected to have differing metabolic activity with corresponding differences in rates of translation and activity of the translational product, and our results may reflect such differences.

Production of cellulase (E1) in transgenic plants has been previously reported in tobacco (Ziegelhoffer et al, ibid, (2001)), Arabidopsis (Ziegler et al, ibid, (2000)) and Potato (Dai et al., Mol. Breeding: 6 pp. 277-285 (2000)). Ziegler et al. (ibid, 2000) examined the expression of E1 catalytic domain that targeted to the apoplast in Arabidopsis thaliana and observed ca. 26% of E1 accumulation in the apoplasts. In our case, the estimated E1 protein accumulation using the same construct in transgenic maize plants was up to 2.1% of total soluble protein. This is several fold lower than that reported in Arabidopsis (Ziegler et al, ibid, 2000), but is in the range reported in transgenic tobacco (Dai et al., Transgenic Research: 14(5): pp. 627-643 (2005)) and in transgenic potato plants (Dai et al, ibid, 2000). However, the exact comparison is difficult as the method of gene transfer and recipient plants are different.

Production of cellulases in a widely grown cultivated maize is important, as maize is considered the main U.S. biomass crop. This study for the first time shows that the biologically active cellulases such as E1 could be produced within the maize biomass at a relatively high level.

### EXAMPLE 14

This example shows the conversion of glucan (cellulose), rice and corn biomass into glucose using rice and corn-produced cellulase.

At present ethanol is mainly produced from corn kernel or corn starch, and today ethanol contributes to about 2% of the total of our transportation fuels mix. The goal of the DOE is to replace 30% of the total transportation petroleum fuel with biofuels by 2025. Since 2000, the repeated recommendations of the National Research Council have been to use crop biomass to supply most of these shortages in transportation liquid fuels because the current supply of sustainable global biomass energy potential is at about 10³⁰ joules per year, in which over 60% is not presently used.

Crop biomass is composed of crystalline cellulose embedded in a hemicellulose and lignin matrix. Cellulose and hemicellulose (called cellulose) are made of chains of fermentable sugars. In order to break the chemical bonds between the cellulose sugars, first one of the pretreatment methods are used to disrupt the biomass and to remove the lignin from the lignocellulosic matter, and then microbial cellulase enzymes are used to convert the cellulose into fermentable sugars for ethanol production.

Despite all efforts, the present technology still cannot economically convert the crop biomass into ethanol because of (1) the still very high cost of production of hydrolytic enzymes in deep microbial tanks, and (2) the costly operation of pretreatments of biomass to disrupt the lignocellulosic matter and remove lignin to help the exposure of cellulose to cellulase enzymes.

To reduce both of the above costs, one could design and sustainably produce cellulase and ligninase enzymes within the crop biomass in manners that these enzymes would remain in their biological active forms for further use, and would not harm the crop growth and development. Then recover and use these enzymes in pure or in crude forms before or during the biorefining process. The plant-produced ligninase can degrade lignin of biomass into phenolic compounds, and the plant-produced cellulase can convert cellulose into fermentable sugars.

Production of these enzymes in plants are possible since plants have been used as “green bioreactors” for the production of essential enzymes and many other proteins, carbohydrates and lipids while requiring minimal inputs of raw materials and energy. Production of biomolecules in plants, considered as molecular farming, is an ideal approach to improve the economics and increase the low-cost production efficiency of the cellulase and ligninase biomolecules.

For example, laccase (ligninase) has been produced under a seed specific promoter in seeds of maize and Acidothermus cellulolyticus endo-1,4-β-D-glucanase (E1; cellulase) has been constitively produced in the whole (in biomass as well as in the seeds) alfalfa, potato, tobacco, and Arabidopsis plants.

Project Goals and Specific Objectives

Cellulase enzymes which convert lignocellulosic matter into fermentable sugars for ethanol production are presently produced in microbial tanks at about $0.34/gallon of ethanol. Pretreatments are also performed to remove lignin from the lignocellulosic matter at a similar casts. The inventor's goal is to produce the cellulase and ligninase enzymes within the crop biomass cells in their biologically active forms at a level needed for such conversion of cellulose into fermentable sugars and lignin into phenolics at costs of $0.03/gallon of ethanol. To achieve this goal, the inventor will reach the following specific objectives.

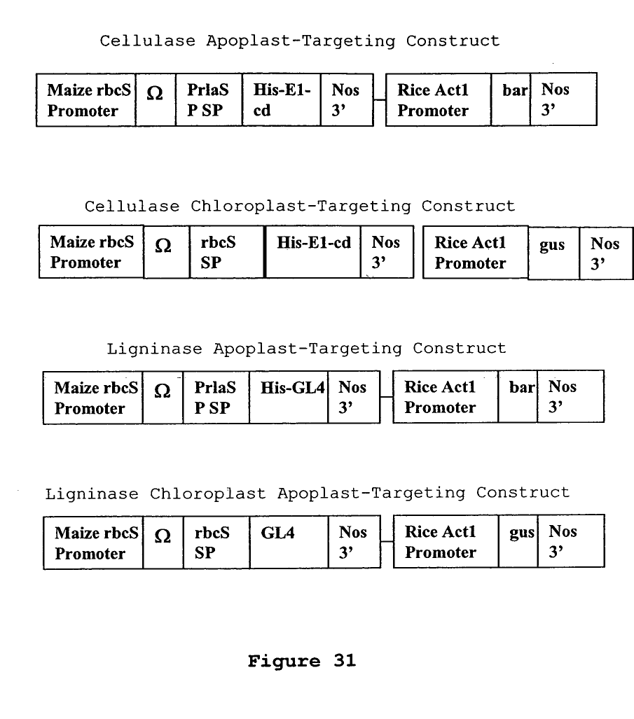

Develop a series of constructs containing the same thermostable celluluse (E1) used in her previous research, and the ligninase genes (CGL4) designed to be targeted into plant apoplast and chloroplast of the same or different plants (FIG. 31).

As rice is the ideal cereal model crop and easy to genetically transgorm, we will also develop Ti plasmid vectors similar to the one in FIG. 21, containing the E1 and CGL4 genes designed for apoplast and chloroplast targeting for rice genetic transformation.

Genetically engineer the model plant, rice with Agrobacterium containing each of the constructs.

Produce corn immature embryos in greenhouses and shoot apical multi-meristem primordial in laboratories, and genetically engineer these corn explants via the co-transformation technology using each of the above constructs and a construct containing a strong corn-specific selectable marker gene regulated by a strong developmentally controlled constitutive promoter, using the Biolistic gun device.

Grow a minimum of 10 independent transgenic plants (each with at least 10-20 plants) from each construct used, and confirm transgene copy number, integration and transcription via molecular techniques.

Develop more antibodies to E1 and to CGL4 enzymes, and confirm the translation of E1 and CGL4 in transgenic plants via Western blotting, and the localization of the enzymes in plant cell compartments via immunoflourescent confocal laser microscopy.

Extract transgenic plant total soluble proteins, and measure the enzymatic activity and determine the percentage of biologically active E1 and CGL4 in plant total soluable proteins via UV spectrophotometry.

Cross breed plants producing the highest level of E1 for maximum enzyme yield, and self breed for production of homozygous transformants.

Approaches:

Plasmid Construction: A set of four different constructs are made. The first; use the catalytic domain E1-tagged to coding sequences for a polyhistidine-regulated by rubisco promter, the tobacco mosaic virus translational enhancer, Nos terminator and the sequences encoding tobacco pathogenesis-related protein 1a (Pr1a) signal peptides for the targeting of E1 into the plant apoplast. The Second will be similar to the first except, we will use the maize rubisco transit peptide to direct the E1 into the chloroplast instead of its targeting into apoplast. The third construct will be similar to the first, except will use the CGL4 ligninase coding sequences instead of the E1. The fourth will be similar to the third construct, except we will use the maize rubisco transit peptide (rbcS SP) for targeting of the CGL4 into the chloroplast instead of apoplast.

FIG. 31 illustrates a schematic representation of plasmid vectors containing two cassettes, one containing the Acedothermus cellulolyticus E1 catalytic domain or the ligninase (CGL4) driven by maize rubisco promoter (rbcS) or Cauliflower Mosaic Virus 35S Promoter (CaMV 35S), tobacco Mosaic Virus translational enhancer (Ω), and the sequence encoding the tobacco pathogenesis-related protein 1a (Pr1a) signal peptide for apoplast-targeting or the maize rbcs signal peptide for enzyme targeting, and the polyadenylation signal of nopaline synthase (Nos). The second cassette contains either the bar selectable maker gene or the gus color marker gene regulated with the rice actin 1 promoter and intron (Act1) promoter and the Nos terminator. The constructs containing the gus gene will be usedc in a 1:1 ratio with a construct containing the bar for chemical selection. The above plasmids will be used alone or in combination to transform maize (Table 13 below).

TABLE 13
Transformation options to target E1 and/or CGL4 to apoplast
and/or chloroplast

| Transformation | | | | | | | | |
|---|---|---|---|---|---|---|---|---|
| Options | I | II | III | IV | V | VI | VII | VIII |
| E1 to Apoplast | * | | * | | | | * | * |
| E1 to | | * | * | | | | * | * |
| Chloroplast | | | | | | | | |
| CGL4 to | | | | * | | * | | * |
| Apoplast | | | | | | | | |
| CGL4 to | | | | | * | * | * | * |
| chloroplast | | | | | | | | |

Corn is not easily amenable to different chemical selections (antibiotics, etc). Because the bar gene is the ideal selectable marker gene known and it is routinely used by us since late 1980's in corn transformation, we will only use bar with an understanding that the use of gus color marker and the routine use of the PCR will select transgenic plants that contain 2-3 of the above gene constructs. To use more than one plasmid, the desired plasmids will be equally mixed (ratios of 1:1, 1:1:1 and 1:1:1:1) and biolistically bombarded into the maize explants in co-transformation fashion as will be explained in the next section.

The 35S can be used instead of the rbcS promoter, because our research has already shown high level production of E1 with the use of the 35S promoter. The construction of the above will follow the work performed in our research as shown in production of constructs with E1 for use in rice (FIG. 26) and in maize transformation. The first cassette of each of the above four plasmids will be inserted in pTi binary vector pCAMBIA 3301 (Ti border vector) as we did for transforming rice with the E1, gus and bar transgenes (FIG. 26).

Production of corn immature embryo-derived cell lines and the shoot apical multi-meristem primordial, transformation of rice via the Agrobacterium system and Co-transformation of corn via the Biolistic® gun:

Corn seeds can be germinated and plants can be grown in greenhouses to maturity. Immature embryos can be collected and cultured in appropriate medium supplemented with appropriate growth regulators. Corn shoot apical multi-meristem primordial can also be developed as per our previous patented technology.

Rice can be transformed using the Agrobacterium vector containing each of the above plasmids following our routine rice transformation research (FIG. 26).

For corn transformation, the corn embryogenic cell lines and multi-meristems can be bombarded with tungsten particles coated with each plasmid containing both the gene of interests and the bar or the gus genes, or can be co-transformed (in equal ratios) with the plasmids containing the gene of interest and the bar selectable marker or the gus color marker gene. The bombarded explants can be selected in medium containing 6-mg/L glufosinate ammonium (PPT) and or can be tested via the Gus colorometric assay, and plantlets can be developed and transferred to greenhouses for testing, self and cross breeding, and seed production. Plants with different transgene expression can be cross bred to see whether both E1 and CGL4 could be produced at high levels in one plant.

Confirmation of integration, copy number and expression of transgenes in corn plants: Polymerase Chain Reaction (PCR) can be used to confirm the presence of the foreign genes in plants. Those shoots/plantlets, which show positive PCR signals, can be further tested for copy number via Southern blotting, for transcription via Northern blotting, and for translation via Western blotting.

Identification of the level of heterologous E1 enzyme production and the biological activity of this enzyme produced in corn plants: The E1 biological activity test can be performed as we have done previously. Soluble proteins can be extracted from leaf tissues by grinding in the sodium acetate buffer and precipitating with ammonium sulfate and quantifying using the BioRad (Hercules, Calif.) Protein Dye Reagent as specified by the manufacturer. The fluorescence can be read at 465 nm using SPECTRAmax M2 device (Molecular Devices Inc., Sunnyvale, Calif.) at an excitation wavelength of 360 nm. After subtracting background fluorescence contributed by the control, activity of samples can be calculated using a standard curve representing 4 to 160 pmol MU and compared to the activity of pure E1. The Ligninase activity test can be confirmed as described (Tien et al, ibid, 1988; Boominathan et al., 1990).

Study of localization of heterologous E1 in transgenic corn cell compartments: We can confirm the localization of the heterologous E1 and CGL4 in apoplast of transgenic corn leaves using a monoclonal antibody specifically raised for this enzyme, as we performed in our apoplast-targeted E1 experiment. We can also confirm the targeting of E1 and CGL4 in chloroplasts by first isolating chloroplasts and using the same E1 antibody against these chloroplasts following our previous work on localization of three polyhydroxybutyrate enzymes (PHB) in transgenic corn chloroplasts (Teymouri F., Alizadeh H., Laureano-Preze L., Dale B., and Sticklen M B (2004). Effects of Ammonia fiber explosion (AFEX) on the activity of heterologous cellulose enzyme. Applied Biochemistry and Biotechnology. 16: 1183-1192.).

Testing the conversion of glucan into glucose and the lignin to phenolics using the corn-produced heterologous E1 and CGL4 enzymes:

Plant total soluble proteins can be extracted, and the amount of E1 in the total soluble proteins can be measured with the fluorescence dye assay using a UV spectrophotometer.

β-glucosidase can be added in each experiment in order to avoid cellabiose inhibition. The reactions are performed at 65° C. with 90 rpm, in a shaking incubator. Sampling can be done after 24, 48 and 72 hrs and checked for the amount of glucose using HPLC, Aminex 87P column against sugar standards as performed by our team before.

The plant produced CGL4 can be tested against commercially available ligninase.

Self breeding, and cross breeding of transgenic plants: Corn breeding is a routine in the inventor's facilities, and can take place in her greenhouses. Rice has been used as a model plant for corn genetic transformation because genetic engineering of rice is far less time consuming and easier than genetic engineering of corn. Therefore, while producing the thermostable A. cellulololyticus E1 in corn, she developed a rice-specific vector and conducted another experiment producing the thermostable E1 (apoplast tragetted), the gus and the bar herbicide resistance genes (FIG. 21) in rice via the Agrobacterium system (see FIGS. 22A,B and FIGS. 23A-D). In this research, rice produced the biologically active E1 up to 4.9% of plant total soluble proteins. She also found that the rice produced E enzyme was biologically active and could convert about 30% of rice biomass into glucose (FIG. 26) using Ammonia Fiber Expossion (AFEX) pretreated rice straw (Dr. Bruce Dale's laboratory).

FIG. 21 shows the schematic representation of ApoE1 binary vector containing the Acedothermus cellulolyticus E1 catalytic domain driven by Cauliflower Mosaic Virus 35S Promoter (CaMV 35S), tobacco Mosaic Virus translational enhancer (Ω), and the sequence encoding the tobacco pathogenesis-related protein 1a (Pr1a) signal peptide for apoplast-targeting of the E1 enzyme, and the polyadenylation signal of nopaline synthase (nos).

FIG. 22 illustrates genetic engineering of rice using the construct shown on FIG. 21 above. FIG. 22A shows Gus expression in plantlets of transgenic rice as compared to the untransformed control. FIG. 22B illustrates greenhouse grown E1 transgenic rice plants. FIG. 23 shows A) PCR, B) Southern, C) Northern and D) Western Blot analyses showing the presence and expression of the transgenes in five transgenic rice lines.

FIG. 26 illustrates in panel FIG. 26A the amount of glucose released from the enzymatic hydrolysis of CMC (1%, 5%, 10%) and Avicel (1%, 5%, 10%) using total protein extracted from E1 expressed rice straw. In panel (b) Comparison of percentage of glucan converted in the enzymatic hydrolysis of corn stover (CS) and rice straw (RS). CE, commercial enzyme, UT, untreated biomass, CS1, RS1, CS2, and RS2 represent, reaction done using 0.5 ml and 4 ml of total protein (with 4.9% of E1) and commercial β-glucosidase (6.5 mg/15 ml) respectively.

Please note that although we used AFEX as the pretreatment choice, we could in future use any other methods of pretreatment (for example, acid and/or heat used at NREL) because we have added the rice produced E1 after the pretreatment to convert glucan into glucose.

Our team also used the E1 cassette from the FIG. 1 and a construct containing the bar herbicide selectable marker genbe regulated by rice actin promoter and intron (FIGS. 4a and 4b below), co-transformed corn and constitutively produced this E1 in maize in an apoplast targeting manner (FIGS. 5a and 5b).

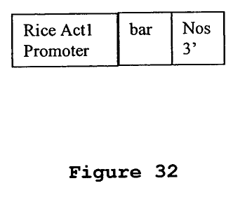

FIG. 27 and FIG. 32 are schematic drawings of two plasmids used in 1:1 ratio combination in maize transformation experiments. In these constructs, Ω represents for the tobacco Mosaic Virus translational enhancer, Pr1a SP for the sequence encoding the tobacco pathogenesis-related protein 1a signal peptide for apoplast-targeting of the E1 enzyme, Nos for the polyadenylation signal of nopaline synthase, and bar for the herbicide resistance sequences.

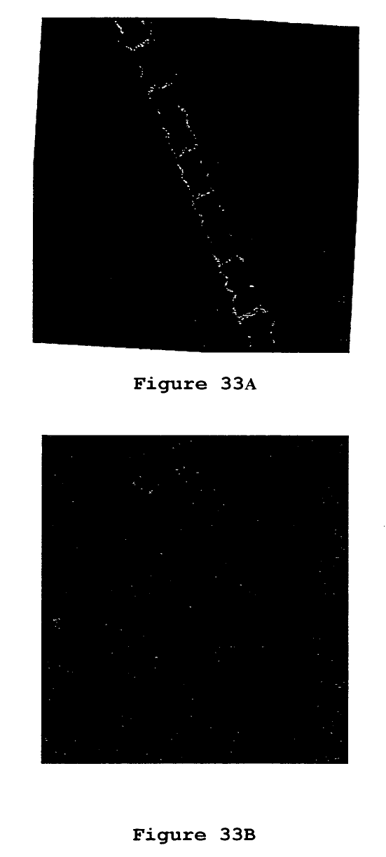

FIG. 33 shows immunoflourescent confocal laser microscopy of apoplast-targeted E1 transgenic maize leaf tissue (left) using the E1 primary antibody and the FITC anti-mouse secondary antibody. Photo on right is leaf tissue from an untransformed control maize plant.

FIG. 30 is a Western blot of transgenic maize plants (1 μg total soluble protein) expressing E1-cd. Lanes +C, positive transgenic tobacco control; −C, untransformed maize control; 1, 2, 5 and 6 transgenic maize plants.

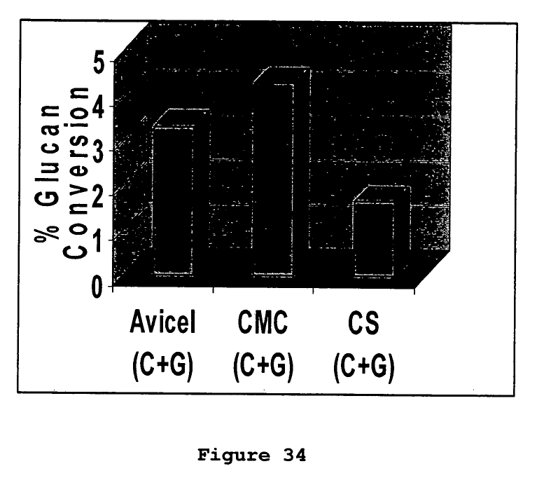

Furthermore, the E1 enzymes produced within the biomass was also biologically active and could convert AFEX pretreated corn stover, and commercially available Avicel and CMC into glucose (FIG. 34). Glucan converted into glucose via the enzymatic hydrolysis of pretreated corn stover (CS), Avicel and CMC using maize-produced E1. Comparison of percentage of conversion to glucan is shown.

Transformation and Heterologous Enzymes targetting in maize cell compartments is a routine in our laboratory. We have also genetically engineered maize with three different polyhydroxybutyrate pathway key enzyme coding sequences while targetting these enzymes to maize chloroplasts (FIG. 7; Zhong et al, 2003).

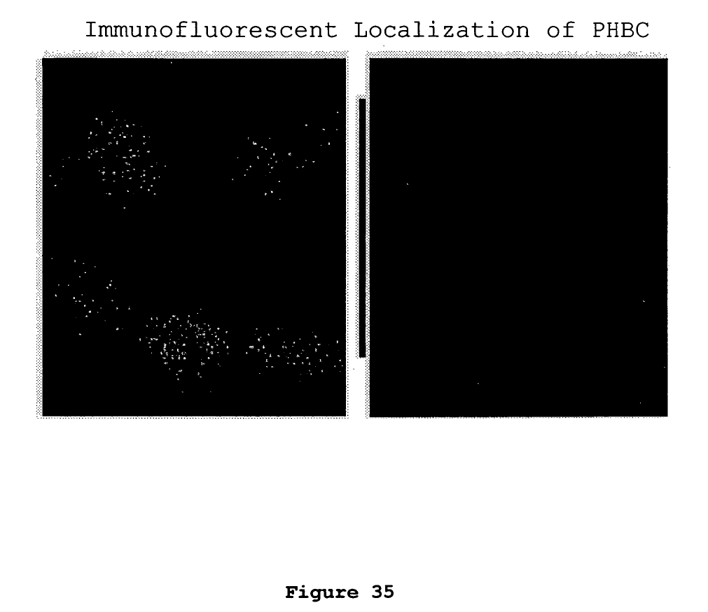

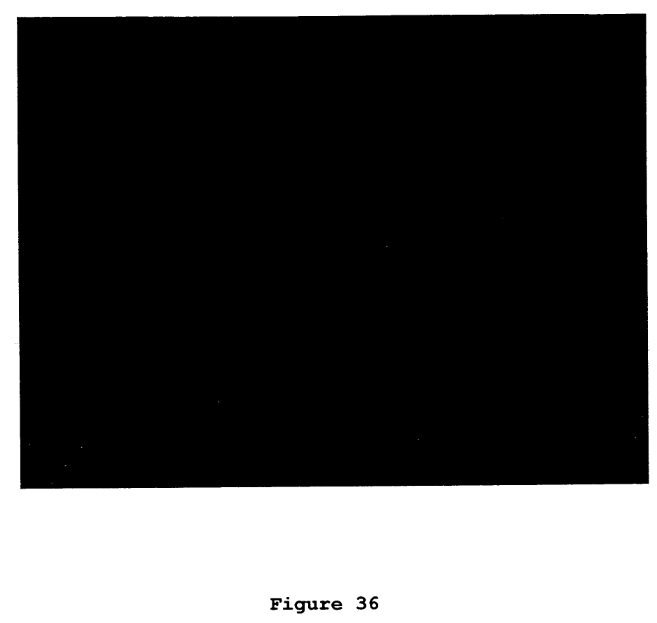

FIG. 35 shows immunoflourescent confocal laser microscopy of chloroplast-targeted polyhydroxybutyrate C in transgenic maize leaf tissue (left) using the PHBC primary antibody and the FITC secondary antibody. Photo on right is leaf tissue from an untransformed control maize plant.

With our work on E1 transgenic rice and corn, there are three possible explanations why the heterologous E1 accumulated in apoplast did not harm transgenic plant cell walls. First, lignocellulose is difficult to hydrolyze because it is associated with hemicellulose, and surrounded by a lignin seal, which has a limited covalent association with hemicellulose. Moreover, cellulose has a crystalline structure with a potential formation of hydrogen bonds resulting in a tightly packed structure with less access to hydrolytic enzymes. Second, cellulases function in a synergistic enzyme complex. If only one enzyme of the complex is expressed such as E1, this single enzyme should not be sufficient to significantly affect the integrity of the cell wall by itself. Third, due to the thermophilic nature of the E1, the enzyme has limited activity under plant in vivo temperature.

We have accumulated the thermostable E1 in apoplast rather than keeping the enzyme in cytosol, where it is produced. There are two advantages associated with production of the E1 and other heterologous enzymes outside of cytosol. First; the foreign enzyme will not harm the cell by interfering with its cytosolic metabolic reactions. Second, the enzyme can accumulate at a much higher level in these compartments as compared to its production in cytosol (i.e. maximum of 0.1-0.3% of plant total soluble proteins). For example, E1 was accumulated in apoplast of transgenic Arabidopsis as high as 26% of plant total soluble proteins (Ziegler, M. T., Thomas, S. R. & Danna, K. J. (2000). Accumulation of a thermostable endo-1,4-β-D-glucanase in the apoplast of Arabidopsis thaliana leaves. Mol. Breed. 6, 37-46).

### EXAMPLE 15

Despite the very successful efforts on reducing the costs of production of fermentation tank microbial hydrolysis enzymes, the relatively high costs of these enzymes are still a barrier to commercial conversion of biomass into biofuel ethanol. Also, to compete economically with petroleum refineries, fuel ethanol “biorefineries”, especially those based on lignocellulosic materials must make higher value, lower volume products. These high value products will significantly improve profit margins and thereby make attractive the very large capital investments required for ethanol biorefineries. Valuable proteins such as industrial enzymes including the hydrolysis enzymes produced within the plants themselves using genetic engineering would fit quite naturally into an ethanol biorefinery and might be recovered before or during the biorefining process. Industrial enzymes are worth at least ten times as much as ethanol product (per unit mass). The present invention provides these enzymes within the maize biomass cells in their biologically active form at a level sufficient for such conversion.

Maize biomass cell walls are composed of approximately 36.1% cellulose, 29.2% hemicellulose (including 21.4% xylan, 3.5% Arabinan, 2.5% galactan and 1.8% mannan) and 17.2% lignin. This means that to convert most of the maize lignocellulosic matter, one might need to hydrolyze the plant biomass with cellulase, hemicellulase, and ligninase enzymes. Three Cellulases including endoglucanase, exoglucanase and the β-glucosidase are involved with hydrolysis of cellulose. Similar combination of hemicellulases is involved with conversion of hemicellulose into fermentable sugars. Our team has already produced biologically active thermostable Acidothermus cellulolyticus. endo-1,4-β-D-glucanase (E1) in maize biomass at a level sufficient as compared to the amount of commercial endoglucanase presently added to the plant biomass for glucan conversion. Work is also in progress our laboratory for production of Trichoderma reesei exoglucanase (CBH1) in maize biomass, as CBH1 plays a synergistic effect on E1 for cellulose hydrolysis. Because CBH1 might not sustain its biological activity in transgenic plants, we are examining the effect of a histidine-tag and a molecular chaperon on activity of CBH1 in tobacco, and then in maize to test the easy purification and correct protein folding of this enzyme. With collaboration of Dr. Bruce Dale of Michigan State University, we also found that about ⅔ of the E1 enzymatic activity in transgenic plants is lost due to Ammonia Fiber Explosion (AFEX) pretreatment (Teymouri F., H. Alizadeh, L. Laureano-Preze, B. Dale, and M. B. Sticklen (2004). Effects of Ammonia fiber explosion (AFEX) on the activity of heterologous cellulose enzyme. Applied Biochemistry and Biotechnology. 16: 1183-1191; Dale B E., Leong C K., Pham T K., Esquivel V. M., Rios I., and Latimer V. (1996). Hydrolysis of lignocellulosics at low enzyme levels: Application of the AFEX Process. Biosource Technology. 56: 111-116). However, separation of E1 in transgenic plant total soluble proteins and addition of such protein to AFEX pre-treated dry biomass could replace the need for commercial E1 (Oraby H., Ransom C., Venkatesh B., Dale B., and Sticklen M. B. (2005). High level production of biologically active thermostable Acidothermus cellulolyticus endo-1,4-β-D-glucanase (E1) in rice biomass converting maize and switchgrass biomass into fermentable sugars. Plant Biotech. Ready for submission).

All hydrolysis enzymes in their active forms are produced in the same or in different maize plant biomass, and these plant made enzymes are used for biomass conversion. The Butyrivibrio fibrisolvens β-glucosidase (Yao, ibid, 2004), Cochliobolus carbonum endoxylanase (Apel P. C., Panaccione D. G., Holden F R., J. D. Walton (1993) Cloning and targeted gene disruption of XYL1, a β-1,4-xylanase gene from the maize pathogen Cochliobolus carbonum. Mol Plant-Microbe Interact 6: 467-473), and the white rot filamentous Phanerochaete chrysosporium ligninase (de Boer H. A., Zhang Y. Z., Collins and C. A. Reddy (1988). Analysis of nucleotide sequences of two ligninase cDNAs from a white-rot filamentous fungus, Phanerochaete chrysosporium. Gene: 69(2):369) are produced in maize biomass. Each of these enzymes are produced separately in maize, and then plants expressing the highest level of each biologically active enzyme are cross bred to produce some or all of the above in one plant without any apparent effect on transgenic plants growth and development. In the case where enzyme combinations cause damage to the maize plant cell walls, each heterologous enzyme can be isolated (in plant total soluble proteins) to be added to the biomass after pretreatment. The Arabidopsis Flowering Locus C (flc) is used in maize for delay in flowering and increase in biomass as was observed in FLC-transgenic tobacco (Salehi H., Ahmad R., Ransom C., Kravchenko A., and Sticklen M. B. B. (2005b). Convergence of goals: Expression of flowering locus C in an Acidothermus cellulolyticus endo-1,4-β-D-glucanase producing tobacco (Nicotiana tobacum L.) for delay in flowering, increase in biomass and phytoremediation of lead. Appl. Biochemistry & Biotechnology. Submitted). The delay in flowering can reduce cross contamination of transgenes with other maize plants in the field, should maize be planted in different fields at more or less the same time

Petroleum refineries make very large-scale, low cost products (gasoline, diesel fuel, kerosene, etc.) that provide the economic “muscle” to compete in the marketplace while their profitability is greatly enhanced by higher value-lower volume products such as chemicals, plastics and solvents. If we are to develop “biorefining” industries based on renewable plant materials, we must simultaneously develop large-scale products such as fuel ethanol along with higher value-lower volume products such as industrial enzymes including hydrolysis enzymes. Protein co-production with biofuels will help make biorefineries more economically competitive and will lay the foundation for replacing much of our imported oil (at a cost of 6 million barrels per day imported at an average cost of $20 per barrel) with home grown fuels such as ethanol.

Our team has produced the microbial endoglucanase in maize biomass at the level comparable to the need for conversion. The inventor, along with Dr. Bruce Dale at Michigan State University has discovered that the E1 enzyme produced within the plant loses ⅔rd biological activity during the AFEX pretreatment conditions (Teymouri et al. ibid, 2004). Therefore, in one embodiment of the present invention the hydrolysis enzymes are produced within the plants, extracted as total soluble proteins, stored under ideal conditions, and added to the lignocellulosic matter after pretreatment for the enzymatic hydrolysis of the biomass matter.

Flc in transgenic maize: The inventor have recently transferred the Arabidopsis thaliana Flowering Locus C (flc) gene to a non-early flowering tobacco and found up to 36 days of delay in flowering, fertile plants, and an increase in plant biomass as compared to non-transgenic plants (Salehi H., Seddighi, Z., C. Ransom, H. Oraby, R. Ahmad and M. Sticklen (2005a). Convergence of goals: Expression of flowering locus C in an Acidothermus cellulolyticus endo-1,4-β-D-glucanase (E1) transgenic tobacco (Nicotiana tobacum L.) and its effect on delay in flowering, increase in biomass and phytoremediation. Applied Biochemistry and Biotechnology Submitted.). Maize is an open pollinated crop that when genetically transformed, requires at least one method of bioconfinement to assure that its pollen grains will not transfer transgenes to other maize plants in the field (CRC Report, 2004). This flc gene encodes a novel protein that acts as a repressor of flowering. It has been cloned, characterized and confirmed to delay in flowering by about four weeks after it was transferred to an early-flowering Arabidopsis. Also, when this gene was null mutated, plants gave early flowering (Michaels S. and Amasino R., (1999). Flowering Locus C encodes a novel MADS domain protein that acts as a repressor of flowering. Plant Cell. 11(5): 949-956). Herein, the flc gene is transferred to maize (along with hydrolysis and bar genes) to meet all or some of the bioconfinement of transgene flow requirements. The flc gene can work in maize as it did in early-flowering Arabidopsis and non-early flowering tobacco (Salehi et al., ibid, 2005). This delay in flowering (Van-Esbroeck G., Hussey M., and Sanderson (1998), Selection response and developmental basis for early and late panicle emergence in Alamo switchgrass. Crop Sci. 38 (2): 342-346) increases the vegetative production and therefore an increase in plant biomass, as it did in transgenic tobacco (Salehi et al, ibid, 2005).

Maize can be genetic engineered using the Biolistic® gun bombardment of multi-meristem primordia as well as the immature embryo-derived maize cell lines. The genetically engineered plants can be biologically confined to block or reduce the transfer of transgenes to their cross breedable field crops via pollen transfer (Kirk T K, Tien M., Kersten P J., Mozouch M D., and Kalyanaraman B. (1986). Ligninase of Phanerochaete chrysosporium. Mechanism of its degradation of the non-phenolic arylglycerol beta-aryl ether substrate of lignin. Biochem. J. 236: 279-287). One method to accomplish this is to delay the flowering of transgenic plants by 3-4 weeks to bypass the flowering of other maize plants in the field as this is the window of time that maize plants flower in one geographic area when maize seeds are planted in different fields at more or less the same time. For example, we have used this method in tobacco via the transfer of Arabidopsis flc transgene. The delay in flowering also caused a significant increase in plant biomass (Salehi et al. ibid, 2005a, Salehi et al, ibid, 2005b).

Background

Genetic Transformation of plants with hydrolysis enzymes. Northwest National Laboratory and the University of Wisconsin, and University of Colorado and NREL have produced E1 cellulase in alfalfa, potato, tobacco, and Arabidopsis (Dai Z.; Hooker B. S.; Queensberry R. D. and Gao J. (1999). Expression of Trichoderma reesei Exocellobiohydrolase I in transgenic tobacco leaves and calli. Applied Biochemistry and Biotechnology 77/79: 689-699; Ziegler, M., Thomas, S., and Danna, K. (2000). Accumulation of a thermostable endo-1,4β-D-glucanase in the apoplast of Arabidopsis thaliana leaves. Mol. Breed. 6: 37-46; Ziegelhoffer, T., Raasch, J., and Austin-Phillips, S. (2001). Dramatic effects of truncation and sub-cellular targeting on the accumulation of recombinant microbial cellulase in tobacco. Mol. Breed. 8: 147-158, and Prodigene® (College Station, Tex.) produced E1 in maize seeds. With the collaboration of Dr. Bruce Dale of MSU, the biological activity of E1 in transgenic tobacco after Ammonia Fiber Explosion (AFEX) pretreatment was tested and it was found that the enzyme loses two-thirds of its activity during AFEX (Teymouri et al., ibid, 2004). Therefore, the cellulases can be produced in biomass plants, and separated as total soluble proteins before adding to the lignocellulosic matter after the pretreatment. The biologically active Acidothermus cellulolyticus endoglucanase has also been successfully produced by us in rice and maize biomass at a relatively high level (Oraby et al., ibid, 2005).

The following steps are involved in making the transgenic plants of the present invention. First, construct plasmids and bombard the plasmids containing the E1 and CBH1 into corn to produce typically at least 30 independent transgenic lines for each gene. Second, regenerate the R₀ and R₁ transgenic plants, and confirm the integration and expression of transgenes. Third, the localization of E1 and CBHI in transgenic maize cells is examined using immunofluorescent and laser microscopy. Fourth, the production level and activity of E1 and CBHI in transgenic maize plants is determined. Fifth, AFEX (Ammonia Fiber Explosion) pretreatment is performed on transgenic tobacco and maize and then test for retention of cellulase (E1 and CBHI) activity.

These five steps have been successfully accomplished for genetic transformation of the Acidothermus cellulolyticus E1 as described below. The same is performed for the Trichoderma reesei CBH1 transgenesis in corn. We have used the Acidothermus cellulolyticus thermostable E1-transgenic tobacco to test whether this enzyme can sustain its biological activity during the mildest pretreatment conditions (i.e. Ammonia Fiber Explosion (AFEX) system). We discovered (Teymouri et al., ibid, 2004) that this Acidothermus cellulolyticus E1 in E1-transgenic plants lost two-thirds (⅔^{rd}) of its biological activity during AFEX pretreatment. Therefore, we decided to separate E1 as total soluble proteins and then add it to the maize straw after AFEX pretreatment.

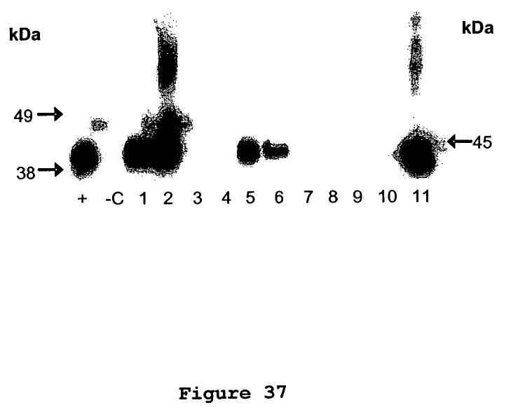

As rice is much easier and quicker than maize to transform, we first transformed rice with of E1 and the bar herbicide resistance genes, then transformed maize with the same transgenes. FIG. 29 and FIG. 37 confirm the integration and translation of microbial E1 in maize lines. Furthermore, FIG. 33A and FIG. 33B, in addition to Table 14 below show the localization of the E1 enzyme in maize leaf cells (FIG. 33A and FIG. 33B) and the biological activity of E1 in maize, rice and tobacco leaf biomass (Table 14).

Cross breeding of E1 transgenic maize: We have made self and crosses between E1 transgenic plants and the control untransformed to test the stability of transgene expression and enzymatic activity in future generations. T1 seeds have been collected for testing.

Preliminary work on apoplast localization of E1 in transgenic maize: Based on our previous experience on localization studies of other gene products (polyhydroxybutyrate) in maize via confocal microscopy, we used E1 primary and a corresponding secondary antibody and performed localization of E1 in transgenic plant apoplast. One sample showed possible localization of E1 in apoplast (FIG. 33A and FIG. 33B). Most samples showed strong non-specific binding of the fluorescence conjugate to plant tissues, however non-specific binding can be blocked using blocking agents.

Level of E1 production and its biological activity in transgenic plants: We measured the E1 enzyme produced and its biological activity in transgenic maize and compared with T₄ tobacco and T₀ rice. The production levels and their activities varied among different transgenic lines due to the transgene position effect. In maize, we obtained E1 up to 9.07%; in rice up to 24.13% and in tobacco up to 3.8% of the plant total plant soluble proteins (see Table 14 below).

TABLE 14
E1 enzymatic activity and percentage of E1 in total soluble
proteins in T₀ maize, T₄ tobacco and T₀ rice.
Maize lines are the same shown in the Western blot of FIG. 37.

| | Activity | % E1 in total |
|---|---|---|
| Plant lines | (nmol/μg/min) | soluble proteins |
| +transgenic | 1.521 | 3.8% |
| tobacco | | |
| Control maize | 0.00 | 0.00 |
| Maize 1-1 | 0.1044 | 0.261% |
| Maize 1-2 | 3.629 | 9.07% |
| Maize 1-4 | 0.0798 | 0.199% |
| Maize 1-6 | 0.0735 | 0.184% |
| Maize 1-10 | 0.0124 | 0.0309% |
| Maize 1-11 | 0.186 | 0.465% |
| Maize 1-13 | 0.0331 | 0.0827% |
| Maize 2-3 | 0.0727 | 0.182% |
| Rice 8 | 9.654 | 24.134% |

The hydrolysis enzymes in their active forms can be produced in the same or in different maize plant biomass to use for biomass conversion. Producing hydrolysis enzymes within the plant biomass can lead to cheaper enzyme production. Acidothermus cellulolyticus thermostable E1-transgenic tobacco was used to test whether this enzyme can sustain its biological activity during the mildest pretreatment conditions (i.e. Ammonia Fiber Explosion (AFEX) system). We discovered (Teymouri et al., ibid, 2004) that this Acidothermus cellulolyticus E1 in E1-transgenic plants lost two-thirds (⅔^{rd}) of its biological activity during AFEX pretreatment. Therefore, we decided to separate E1 as total soluble proteins from transgenic plants, and add to the maize straw after AFEX pretreatment.

New maize seeds are germinated and grown in greenhouses to maturity, then the immature embryos are collected and immature embryo-derived cell lines are produced in vitro for Biolistic® bombardment. New shoot apical meristem primordia are produced, multiplied by thousands through biweekly subculture for bombardment via the Biolistic® method as described in Zhong H., F. Teymouri, B. Chapman, S. Maqbool, R. Sabzikar, Y. El-Maghraby, B. Dale, and M. B. Sticklen. (2003). the dicot pea (Pisum sativum L.) rbcS transit peptide directs the Alcaligenes eutrophus polyhydroxybutyrate enzymes into the monocot maize (Zea mays L.) chloroplasts. Plant Sci. 165: 455-462, Zhang H., D. Warkentin, B. Sun, H. Zhong, and M. B. Sticklen (1996). Variation in the inheritance of expression among subclones for unselected (uidA) and selected (bar) transgenes in maize (Zea mays L.). Theoretical and Applied Genetics. 92: 752-761. The system of shoot apical multimeristem bombardment, reproduction, and propagation has previously been developed for maize nuclear transgenesis as described also in U.S. Pat. Nos. 5,281,529; 5,320,961; 5,767,368 to Zhong et al., hereby incorporated herein by reference.

A set of plasmids is constructed containing: (1) Cochliobolus carbonum endoxylanase (Apel et al., 1993), (2) Butyrivibrio fibrisolvens β-glucosidase (Yao, 2004), and (3) the white rot filamentous Phanerochaete chrysosporium ligninase (de Boer et al., ibid, 1988). Each enzyme gene is regulated by the 35S promoter, tobacco mosaic virus translational enhancer, and the sequences encoding the tobacco pathogenesis-related protein 1a for the targeting of each of the enzyme gene products into the plant apoplast. In the plant apoplast, the pH is acidic and falls between approximately pH 5 to pH 5.5. The ideal pH for enzymatic activity of the enzymes fall between approximately pH 4.5 to pH 5.2. Therefore, the apoplast is an ideal storage location for sustaining the enzymatic activity of each of the enzymes.

The maize immature embryo-derived cell lines and multi-meristem primordia are co-transformed with each of the above three constructs along with another construct (for example pGreen) containing the bar herbicide resistance selectable marker and the Arabidopsis Flowering Locus C (FLC) genes in a manner that each plant will express one of the three hydrolysis genes, bar and flc. Next, greenhouse grown transgenic plants are produced from the co-transformed maize. The integration, copy number, transcription and translation of the above transgenes is confirmed in the transgenic plants by methods well known in the art. The delay in flowering and increase in the biomass of FLC-transgenic plants is then measured. The level of each of the heterologous enzymes is identified as percent of plant total soluble protein. The enzymatic activity of each of the three proteins in each of the transgenic plant lines is measured using specific substrates by enzymatic assay methods well known in the art. The localization of each of the three heterologous protein enzymes within the plant cells is confirmed via the use of specific primary and secondary antibodies followed by confocal microscopy as described for PHB-transgenic maize (Zhong et al., ibid, 2003) and E1 transgenic maize.

Thus, the procedure to provide the transgenic plants of the present invention include: (1) production of highly regenerable multi-meristems and immature embryo callus lines for bombardment; (2) development of three constructs, containing the xylanase, β-glucosidase and ligninase transgenes; (3) co-transformation of 5-10 independent transgenic maize lines, each containing a combination of bar selectable marker and flc, and either the β-glucosidase, xylanase or ligninase transgenes; (4) studying gene integration and transgene copy number via Southern blots, and gene expressions via northern and western blots; (5) studying translation level, enzymatic activity and localization of enzymes in cell maize cells; (6) Studying delays in flowering and increase in biomass of FLC-transgenic maize; and (7) self breeding, and cross breeding of T₀ plants that show the highest level and activity of heterologous enzymes with E1 transgene plants.

Methods:

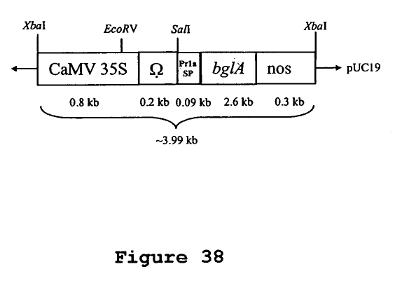

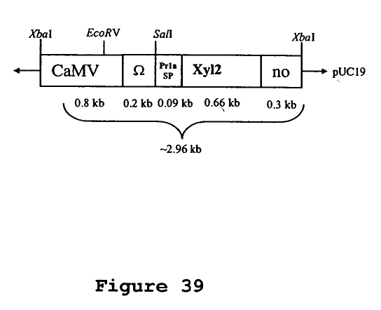

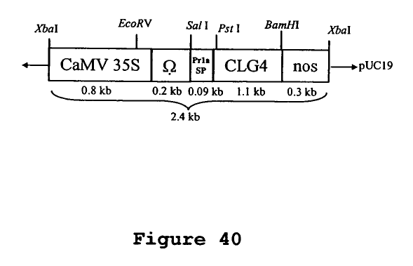

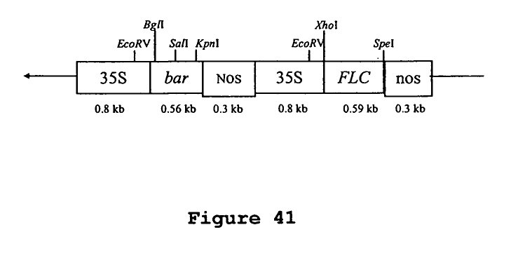

Plasmid Constructions. Construction of plasmids containing each of the β-glucosidase, xylanase and ligninase genes. Develop three new plasmid constructs (FIG. 38, FIG. 39 and FIG. 40); containing the β-glucosidase (FIG. 38), xylanase (FIG. 39), and the ligninase (FIG. 40) coding sequences regulated by the same regulatory sequences as used to produce E1-transgenic maize.

FIG. 38 illustrates a schematic of the plasmid containing the Butyrivibrio fibrisolvens β-glucosidase (Yao J Q (2004). Genetic Transformation of Tobacco with a Beta-Glucosidase Gene to Induce Constitutive Systemic Acquired Resistance Against Tobacco Mosaic Virus. Ph. D. dissertation. Department of Biological Sciences, Western Michigan University) cDNA regulated by the 35S promoter and enhancer. This construct also contains the sequences encoding the tobacco pathogenesis-related protein 1a (Pr1a) signal peptide for targeting of β-glucosidase enzyme into plant apoplast. CaMV 35S: Cauliflower Mosaic Virus 35S Promoter. Ω: Tobacco Mosaic Virus Ω translational enhancer. Pr1a SP: the sequence encoding the tobacco pathogenesis-related protein 1a (Pr1a) signal peptide. bglA: Butyrivibrio fibrisolvens β-glucosidase (Lin L., E. Rumbak, H. Zappe., J A Thompson and D. R. Woods (1990). Cloning, sequencing and analysis of expression of a Butyrivibrio fibrisolvens gene encoding a β-glucosidase. The J. Gen. Microbiol. 136: 1567-1576).

FIG. 39 illustrates a schematic of the plasmid containing the xylanase cDNA regulated by the 35S promoter and enhancer. This construct contains the sequences encoding the tobacco pathogenesis-related protein 1a (Pr1a) signal peptide for targeting of Cochliobolus carbonum endoxylanase (Apel et al., ibid, 1993) into plant apoplast. Abbreviations: CaMV 35S: Cauliflower Mosaic Virus 35S Promoter; Ω: Tobacco Mosaic Virus translational enhancer; Pr1a SP: the sequence encoding the tobacco pathogenesis-related protein 1a (Pr1a) signal peptide; Xyl2: Cochliobolus carbonum endoxylanase cDNA.

FIG. 40 illustrates a schematic of the plasmid containing the Phanerochaete chrysosporium ligninase (de Boer et al., ibid, 1988) gene regulated by the 35S promoter and enhancer. Abbreviations in pMZ766E1-cat: CaMV 35S: Cauliflower Mosaic Virus 35S Promoter; Ω: Tobacco Mosaic Virus Ω translational enhancer; Pr1a SP: the sequence encoding the tobacco pathogenesis-related protein 1a (Pr1a) signal peptide; CLG4: Phanerochaete chrysosporium ligninase; nos: polydenylation signal of nopaline synthase.

Construct for co-transformation with the bar herbicide resistance gene and flc for delay in flowering and an increase in plant biomass: The pGreen, as illustrated in FIG. 41, was obtained from Dr. Richard Amasino of University of Wisconsin and was used in our research on tobacco (Salehi et al., ibid, 2005). pGreen is one example of a plasmid that can be used to co-transform maize with the constructs described above. FIG. 41 illustrates a schematic of the plasmid pGreen which contains the Arabidopsis Flowering Locus C (FLC) coding sequences regulated by 35S promoter and Nos terminator. It also has the bar herbicide resistance selectable marker regulated by 35S promoter and Nos terminator. Abbreviations in pGreen: FLC: Arabidopsis Flowering Locus C coding region; 35S: CaMV 35S promoter; bar: phosphinothricin acetyltransferase coding region.

Production of maize immature embryo-derived cell lines and the apical shoot multi-meristem primordia for genetic transformation: Maize seeds can be germinated and plants grown in greenhouses to maturity. Immature embryos are collected and cultured in Murashige and Skoog (Murashige T. and Skoog F. (1962). A revised medium for rapid growth and bioassay with tobacco tissue culture. Physiol. Plant. 15: 473-497) medium supplemented with appropriate growth regulators for proliferation of embryogenic cell lines as performed for production of E1 in maize. Maize apical shoot multi-meristem primordia can also be developed as previously published (Zhang et al, ibid, 1996; Zhong et al., ibid, 2003).

Maize cell lines are co-transformed via the Biolistic® gun. Embryogenic cell lines and multi-meristems are bombarded with tungsten particles coated with each plasmid and the plasmid containing bar and FLC genes. The bombarded explants are gently transferred onto selection medium containing 6-10 mg/L glufosinate ammonium (PPT) selectable chemical for another six to eight weeks. Chemically selected multi-meristems are further multiplied in selection medium for another three to four months, and the selected immature embryo cells are regenerated into somatic embryos, and germinated into plantlets in appropriate media containing the same concentration of PPT. The selected multi-meristems are rooted in vitro, and seven to ten centimeter plantlets produced from shoots or cell lines are transferred to pots, acclimated, and transferred to maize greenhouses where they are grown to maturity, and T₁ seeds are collected.

Confirming Integration, Copy Number and Expression of Transgenes in Maize Plants:

PCR test: When the selected herbicide resistant plantlets are too many in number for Southern blot analyses, the Polymerase Chain Reaction (PCR) can be used to confirm the presence of the foreign genes in the plants. The shoots and/or plantlets which test positive will be considered as putatively transformed for further molecular analysis.

Southern blot: To find the copy numbers of transgenes in plants, genomic DNA is isolated from greenhouse-grown putatively transgenic and control (untransformed) plants, then Southern blot analysis is performed following our routine modification of Southern's method as we performed for E1 transgene.

Northern blot: To confirm the transcription of transgenes, total cellular RNA is isolated from plant tissues. The mRNA coding the foreign genes is detected by RNA blot analysis using the same probes used in Southern blot hybridization, as above. The mRNA is electrophoresed on a denaturing formaldehyde agarose gel, transferred to nitrocellulose or nylon filters, hybridized with the appropriate probe, and then exposed to X-ray film. After exposure of the probed RNA-containing filter to X-ray film, the hybridization bands can be scanned using a densitometer to determine the levels of specific mRNA present as performed for the transcription of E1 in transgenic maize.

Western blot: Primary polyclonal antibodies can be commercially custom-raised using synthetic peptides. Western blots are performed to find the translation of each transgenes in transgenic plants as performed for E1 heterologous protein.

Identify the level of heterologous enzyme production and the biological activity of each enzyme produced in maize plants. The E1 enzymatic activity and percentage E1 in total soluble proteins in transgenic maize, tobacco and rice has been measured as shown in Table 14. The β-glucosidase biological activity can be performed as described in Fisher, K. and Woods, J. (1999) Determination of β-glucosidase enzymatic function of Histoplasma capsulatum H antigen using a native expression system. Gene: 247 (1-2):191-197. Briefly, supernatants and column elute are electrophoresed in polyacrylamide gels, followed by a thirty minute room-temperature gel wash in enzyme buffer (20 mM Tris-HCl plus 0.6 mM CaCl₂, pH 8.0) and a 37° C. incubation with the β-glucosidase substrate p-nitrophenyl-β-D-glucopyranoside (PNPG) (Sigma-Aldrich®, St. Louis, Mo.). Areas of substrate hydrolysis indicating β-glucosidase enzyme activity are visualized. Individual substrate gels are scanned with an scanner, such as an Agfa® brand (Agfa-Gevaert Aktiengesellschaft, Germany) scanner.

Xylanase activity is measured as described by Apel et al., ibid, 1993. Briefly, the PAHBAH protocol is used. That is, four volumes of 0.5N NaOH is mixed with one volume of 5% PAHBAH stock including the p-hydroxybenzoic acid hydrazide (Catalog no. H-9882, Sigma-Aldrich®, St. Louis, Mo.) in 0.5 M HCl (conc. HCl=12M; 300 ml 0.5 M HCl=12.5 ml conc. HCl) and store for up to one month at 4° C. A standard curve is used (galacturonic acid H₂O M 212.16. 100 ml of 10 mM=0.21 g, or glucose for more intense reaction). The reaction mix is incubated at 37° C. and at each time point (0 and 30 min), 25 μl is withdrawn and added to 1.5 ml of PAHBAH mixture in a 13×100 glass culture tube. The tube is then mixed by flicking or vortexing. Heat the sample to 100° C. for ten minutes and allow it to cool. Then will read absorbance at 410 nm. For large number of samples (e.g. HPLC fractions), a reaction mixture can be made of acetate buffer, substrate and water and then aliquoted in microcentrifuge tubes. The volume of enzyme solution sampled initially and the volume sampled after incubation can be increased if necessary.

Ligninase biological activity is performed as described by Kirk T K, Tien M., Kersten P J., Mozouch M D., and Kalyanaraman B. (1986). Ligninase of Phanerochaete chrysosporium. Mechanism of its degradation of the non-phenolic arylglycerol beta-aryl ether substrate of lignin. Biochem. J. 236: 279-287. The reaction buffer will contain a mixture of 2.2 ml sodium tartrate buffer (50 mM, pH; 405, 40 mM veratryl alcohol (2 mM) and 240 μl of ligninase transgenic plant total soluble proteins. The reaction will be initiated by adding 20 μl H₂O₂ (0.2 mM). The absorbance will be measured immediately with an extinction coefficient ε₃₁₀=9333 M⁻¹ cm⁻¹. The activity will be defined as the quantity of ligninase enzyme that produces 1 μmol of oxidized product.

Study of apoplast localization of heterologous enzymes in transgenic maize. As was previously confirmed for the localization of three polyhydroxybutyrate enzymes (PHB) in maize chloroplasts (Zhong et al., ibid, 2003) and the localization of E1 in transgenic maize apoplast (FIG. 33A and FIG. 33B), commercially raised antibodies can be used against each of the enzymes, and immunofluorescent antibody staining can be performed to determine the localization of the xylanase, ligninase and β-glucosidase in transgenic maize cells. The transgenic leaf section samples are incubated in a solution containing the primary antibody against the xylanase, ligninase or the β-glucosidase. After washing to remove unbound antibodies, the tissues are incubated in the secondary antibody-fluorophore conjugate, which is a goat anti rabbit IgG-Alexa conjugate. After washing to remove unbound antibody-conjugate, the tissues will be mounted on microscope slides and viewed with a laser scanning confocal fluorescence microscope is used to determine the localization of these heterologous enzymes in the transgenic plants.

Confirmation of the FLC heterologous protein production and its effect on delay in flowering and increasing fresh and dry plant biomass production: The FLC has previously been expressed in transgenic tobacco and it was confirmed that this gene could delay flowering up to 36 days while increasing the fresh and dry biomass in the absence of and in the presence of the E1 transgene (Salehi et al., ibid, 2005b; Salehi et al., ibid, 2005b). The expression of FLC in maize is determined via Western blotting. The days for delay in flowering can be then counted as compared to the control plants. Measurements of the plant fresh and dry biomass, thousand seed weight and seed yield (gram per plant) can be taken.

Self breeding and cross breeding is started with transgenic plants showing the highest level and activity of E1 with hemicellulase, ligninase and β-glucosidase transformants. Self or cross breeding is performed with plants for the production of second generation plants, and testing is performed to determine whether combination of enzymes in plants have an effect on plant growth and development. Maize breeding has been described in Zhang et al, ibid, 1996 and Zhong et al, ibid, 2003.

Lignocellulosic biomass is composed of crystalline cellulose embedded in a hemicellulose and lignin matrix. The pretreatment methods are presently used to disrupt the lignicellulosic matter, and to mostly remove the lignin to allow the access of cellulose to cellulases. Plant genetic engineering can decrease lignin and/or change the composition of lignin for less need of expensive and harsh pretreatments. Plant genetic engineering can also produce microbial ligninases within the biomass crops, so the lignin content of biomass could be deconstructed during or before bioprocessing. There are three different groups of cellulases working in concert to convert cellulose into glucose. These enzymes include endoglucanase, exoglucanase and the β-glucosidase. Plant genetic engineering has been successfully used to produce these enzymes in plants. There might be ways to increase biomass via plant genetic engineering. These include genetic manipulation of plant growth regulators or photosynthetic pathways. Delay in flowering also can increase plant biomass.

Production of cellulase enzymes within the crop biomass: As an alternative to its production in microbial tanks, it has been recommended to produce these enzymes within the crop biomass (Sticklen, M, Teymouri, F, Maqbool, S, Salehi, H, Ransom C, Biswas, G, Ahmad, R, and Dale, B: Production of microbial hydrolysis enzymes in biomass crops via genetic engineering. 2nd International Ukrainian Conference on Biomass for Energy 2004, p. 133, 20-22). The apoplast targeting of the translation product of catalytic domain of the gene coding for thermostable Acidothermus cellulolyticus 1,4-endoglucanase E1 enzyme in Arabidopsis tobacco and potato plants demonstrated the possibility of producing this enzyme, in case of Arabidopsis at up to 25% plant total soluble proteins. Recently, the inventor's team constitutively expressed the catalytic domain of the A. cellulolyticus 1,4-endoglucanase E1 in rice (Oraby et al. unpublished) and maize in an apoplast targeting manner. The amount of E1 enzyme produced in rice and maize leaves respectively accounted for up to 4.9% and 2% of the plant total soluble proteins, and the E1 accumulation had no apparent deleterious effects on plant growth and development.

Furthermore, when the crude extract of rice total soluble proteins was added to Ammonia Fiber Explosion (AFEX) pretreated rice straw and maize stover, Approximately 30 and 22% of the cellulose of these plants were respectively converted into glucose (Oraby et al. unpublished, Ransom et al: unpublished).

Initially, there were three concerns associated with production and use of the cellulase enzymes within the crop biomass. The first concern was whether the harsh conditions (acid, alkaline and/or heat.) of pretreatment would destroy the biological activity of these enzymes. The second concern was whether sufficient enzymes could be expressed within the biomass to convert polysaccharides into fermentable sugars without the need for the use of commercial enzymes. The third concern was whether increasing the level of production of these heterologous enzymes within the plant cells would cause harm to plant growth and development.

To address the first concern, the inventor's team used the mildest method of pretreatment i.e. Ammonia Fiber Explosion (AFEX) on the thermostable A. cellulolyticus E1-producing tobacco biomass. In this experiment, about ⅔^{rd} of the activity of this heterologous enzyme was lost. Therefore, the conclusion was to extract the heterologous enzyme in crude or pure forms, and then add to the pretreated matter for production of fermentable sugars. In a follow up study, up to 30% of rice and 22% of maize cellulose were respectively converted into glucose, when the rice-produced E1 was extracted in crude form, stored under freeze condition for 3 months and then added to the AFEX pretreated matter.

To address the second issue, it is possible to increase the level of gene expression by regulating the transcriptional, posttranscriptional and posttranslational factors. However, the increase in production of heterologous proteins is best possible by targeting these proteins from cytosol for accumulation into non-cytosolic cell compartments (Hood E E: Bioindustrial and biopharmaceutical products from plants. In: New directions for a diverse planet: Proc 4^{th} Intl Crop Sci Congress, Brisbane, Austria, Sept. 26-Oct. 1, 2004. ISBN 1 920842 20 9). This by itself would address the third concern because the enzyme accumulation inside these compartments will not interfere with the plant cytosolic metabolic activities.

Another advantage is that there are distinct molecular chaperon systems in targeted compartments to translocate or fold proteins. However, carefully addressing the targeting of heterologous proteins to cell compartments is very important because factors influencing transcription and translation efficiency, recombinant protein accumulation as well as the protein stability strongly depend on the compartment of the plant cell chosen for accumulation.

The question has been asked about the reasons that the E1 heterologous cellulase targeted and accumulated in the apoplast of rice, maize and other plants did not harm the plant cell wall cellulose. The answer is that it might be due to a combination of three reasons. First, heterologous E1 enzyme does not have a direct access to the plant cellulose because cellulose is in a compact mixture along with lignin and hemicellulose. Second, the plant cellulose is in crystalline form, less amenable to hydrolysis by cellulase. Third, the heterologous E1 inartistically from thermophilic A. cellulolyticus might have limited activity at plant in vivo temperature.

The question has also been which compartment should be chosen for targeting of heterologous enzymes? The apoplast targeting enzymes must have a significant influence on the production level rather than on transcription, meaning that the barrier to higher expression in cytosol and chloroplast is post transcriptional. In addition, since the apoplast is the extracellular pathway provided by the continuous matrix of cell walls, it may provide more space than other compartments for a higher level of accumulation.

The chloroplast targeting might require the first 24 amino acids of the mature rbcS protein in addition to the transit peptide. In a maize research (Zhong H, Teymouri F, Chapman B, Maqbool S, Sabzikar R, El-Maghraby Y, Dale B, Sticklen M B: The dicot pea (Pisum sativum L.) rbcS transit peptide directs the Alcaligenes eutrophus polyhydroxybutyrate enzymes into the monocot maize (Zea mays L.) chloroplasts. Plant Sci 2003, 165: 455-462), the first 24 amino acid coding sequence of the mature rubisco small subunit protein was used in addition to the pea rubisco transit peptide to direct three polyhydroxybutyrate pathway enzymes into maize chloroplast.

Targeting of heterologous peptides to vacuole has been performed in several cases. For example, ProdiGene targeted the heterologous laccase into maize seed apoplast (Hood E E: Bioindustrial and biopharmaceutical products from plants. In: New directions for a diverse planet: Proc 4^{th} Intl Crop Sci Congress, Brisbane, Austria, Sept. 26-Oct. 1, 2004. ISBN 1 920842 20 9).

Because targeting of heterologous proteins for high accumulation without harm to plant growth and development has successfully been achieved, a battery of all different groups of polysaccharide and lignin degrading enzymes can be tested within the same crop biomass by targeting each enzyme into the same or different compartments. Also, targeting of the same enzyme into all different cell compartments of the same plant can be tested to maximize the single enzyme production. Therefore, the future of large-scale production of these enzymes within the crop biomass is very bright, and the idea to replace the microbial tank reactors by plants as biofactories for commercial production of these and other industrial enzymes is indeed not impossible.

Flc Gene: Tobacco biomass was significantly increased with nuclear insertion of a single Arabidopsis thaliana Flowering Locus C (flc) gene (Salehi H, Ransom C, Oraby H, and Sticklen M: Delay in flowering and increase in biomass of plants expressing the Arabidopsis floral repressor gene FLC (FLOWERING LOCUS C). Plant Physiol 2005, 162: 711-717). Transfer of a single flc gene that delays flowering can significantly increase the plant biomass, because the energy needed for on time reproduction has shifted into biomass growth. An increase in photosynthesis does not increase the plant biomass, because several other factors such as plant nutrients, oxygen, water, and plant respiration also need to be regulated in order to increase the plant biomass. Increase in photosynthesis also relates to the correct matching of the plant circadian clock period (Dodd A N, Salathia N, Hall A, Kevei E, Toth R, Nagy F, Hibert J M, Miller A J, Webb A A R: Plant circadian clocks increase photosynthesis, growth, survival, and competitive advantage. Science 2006, 309: 630-633) with that of the external light-dark cycle (Sinclair T R, Purcell L C, Sneller C H: Crop transformation and the challenge to increase yield potential. Trends Plant Sci. 2004, 9(2): 70-75). The fact that maize produced 20% more biomass under high CO₂ concentration might be because the C4 maize may have more capacity to synthesize sucrose, starch, and overall biomass under elevated CO₂. This observation needs to be tested in C3 plants.

Plant genetic engineering to improve biomass characterization for a better biofuel economy is a new technology. By definition, a new technology is economically feasible if the social benefits from adopting the technology are greater than its social costs (Communication with Dr. James Oehmke, Plant Biotechnology Economist, Michigan State University). Here, the social costs include costs of resources used without the government subsidies, and the social benefits are the low production costs.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

We claim:

1. A transgenic plant capable of expressing one or more cell wall degrading enzymes comprising:
(a) at least one DNA comprising a cell wall degrading enzyme coding region operably linked to a nucleotide sequence encoding a signal peptide directing the cell wall degrading enzyme encoded by the DNA to a plastid or apoplast of the transgenic plant; and
(b) at least one DNA comprising a flowering locus c gene coding region operably linked to a constitutive promoter, wherein the transgenic plant expresses the one or more cell wall degrading enzymes and a transcription factor encoded by the flowering locus c gene that delays flowering while increasing biomass and enabling isolation of increased amounts of the hydrolyzing enzyme from the transgenic plant as compared to a non-transgenic plant from which the transgenic plant is derived.

2. The transgenic plant of claim 1, wherein the transgenic plant is a monocot.

3. The transgenic plant of claim 1, wherein the monocot is switchgrass, rice or maize.

4. The transgenic plant of claim 1, wherein the one or more cell wall degrading enzymes are selected from the group consisting of a cellulase, a hemicellulase and a ligninase.

5. The transgenic plant of claim 4, wherein the cellulase is an endoglucanase, an exoglucanase or a glucosidase.

6. The transgenic plant of claim 5, wherein the DNA encoding the cellulase is selected from the group consisting of an e1 gene from Acidothermus cellulyticus, a cbh1 gene from Trichoderma reesei, a dextranase gene from Streptococcus salivarius, and a β-glucosidase gene from Actinomyces naeslundi.

7. The transgenic plant of claim 6, wherein the e1 gene comprises the nucleotide sequence set forth in SEQ ID NO:4, the cbh1 gene comprises the nucleotide sequence set forth in SEQ ID NO:10, the dextranase gene comprises the nucleotide sequence set forth in SEQ ID NO:8, and the β-glucosidase gene comprises the nucleotide sequence set forth in SEQ ID NO:6.

8. The transgenic plant of claim 5, wherein the DNA encodes a β-glucosidase from Butyrivibrio fibrisolvens.

9. The transgenic plant of claim 8, wherein the DNA encoding the β-glucosidase comprises the nucleotide sequence set forth in SEQ ID NO:23.

10. The transgenic plant of claim 5, wherein the DNA encodes a ligninase from Phanerochaete chrysosporium.

11. The transgenic plant of claim 10, wherein the DNA encoding the ligninase is ckg4 comprising the nucleotide sequence set forth in SEQ ID NO:11 or ckg5 comprising the nucleotide sequence set forth in SEQ ID NO:13.

12. The transgenic plant of claim 5, wherein the DNA encodes a xylanase from Cochliobolus carbonum.

13. The transgenic plant of claim 12 wherein the DNA encoding the xylanase comprises the nucleotide sequence set forth in SEQ ID NO:24, SEQ ID NO:33, SEQ ID NO:34 or SEQ ID NO:35.

14. The transgenic plant of claim 1, wherein the at least one DNA comprising a cell wall degrading enzyme coding region is operably linked to a leaf-specific promoter.

15. The transgenic plant of claim 14, wherein the leaf-specific promoter is a promoter for rbcS.

16. The transgenic plant of claim 1, wherein the at least one DNA comprising a cell wall degrading enzyme coding region is operably linked to a Cauliflower Mosaic Virus 35S promoter.

17. The transgenic plant of claim 1, wherein the at least one DNA comprising a cell wall degrading enzyme coding region is operably linked to a Tobacco Mosaic Virus Ω translational enhancer.

18. The transgenic plant of claim 1, wherein the nucleotide sequence encoding the signal peptide encodes a signal peptide of rbcS.

19. The transgenic plant of claim 18, wherein the rbcS comprises the nucleotide sequence set forth in SEQ ID NO:1.

20. The transgenic plant of claim 1, wherein the nucleotide sequence encoding the signal peptide encodes a signal peptide of tobacco pathogenesis-related protein 1a (Pr1a).

21. The transgenic plant of claim 1 further comprising at least one DNA encoding a selectable marker operably linked to a constitutive promoter.

22. The transgenic plant of claim 21 wherein the DNA encoding the selectable marker provides the transgenic plant with resistance to an antibiotic, a herbicide, or to environmental stress.

23. The transgenic plant of claim 22 wherein the DNA encoding resistance to the herbicide is a DNA encoding phosphinothricin acetyl transferase which confers resistance to the herbicide phosphinothricin.

24. A transgenic plant capable of expressing cell wall degrading enzymes comprising:
(a) at least one DNA encoding a β-glucosidase as a first cell wall degrading enzyme which is operably linked to a nucleotide sequence encoding a signal peptide directing the β-glucosidase to a plastid or apoplast of the transgenic plant;
(b) at least one DNA encoding a ligninase as a second cell wall degrading enzyme which is operably linked to a nucleotide sequence encoding a signal peptide directing the ligninase to a plastid or apoplast of the transgenic plant,
(c) at least one DNA encoding a xylanase as a third cell wall degrading enzyme which is operably linked to a nucleotide sequence encoding a signal peptide directing the xylanase to a plastid or apoplast of the transgenic plant; and
(d) at least one DNA comprising a flowering locus c gene coding region operably linked to a constitutive promoter, wherein the transgenic plant expresses the cell wall degrading enzymes and a transcription factor encoded by the flowering locus c gene that delays flowering while increasing biomass and enabling isolation of increased amounts of the cell wall degrading enzymes from the transgenic plant as compared to a non-transgenic plant from which the transgenic plant is derived.

25. The transgenic plant of claim 24 further comprising at least one DNA encoding a selectable marker operably linked to a constitutive promoter.

26. The transgenic plant of claim 25 wherein the DNA encoding the selectable marker provides the transgenic plant with resistance to an antibiotic, a herbicide, or to environmental stress.

27. The transgenic plant of claim 26 wherein the DNA encoding resistance to the herbicide is a DNA encoding phosphinothricin acetyl transferase which confers resistance to the herbicide phosphinothricin.

28. The transgenic plant of claim 24 wherein the DNA encoding the β-glucosidase is from Butyrivibrio fibrisolvens.

29. The transgenic plant of claim 28 wherein the DNA encoding the β-glucosidase comprises the nucleotide sequence set forth in SEQ ID NO:23.

30. The transgenic plant of claim 24 wherein the DNA encoding the ligninase is from Phanerochaete chrysosporium.

31. The transgenic plant of claim 30 wherein the DNA encoding the ligninase is ckg4 comprising the nucleotide sequence set forth in SEQ ID NO:11 or ckg5 comprising the nucleotide sequence set forth in SEQ ID NO:13.

32. The transgenic plant of claim 24 wherein the DNA encoding the xylanase encodes an endoxylanase from Cochliobolus carbonum.

33. The transgenic plant of claim 32 wherein the DNA encoding the xylanase comprises the nucleotide sequence set forth in SEQ ID NO:24, SEQ ID NO:33, SEQ ID NO:34 or SEQ ID NO:35.

34. A method for making an enzyme extract comprising one or more cell wall degrading enzymes comprising:
(a) providing a transgenic plant capable of expressing one or more cell wall degrading enzymes comprising at least one DNA comprising a cell wall degrading enzyme coding region operably linked to a nucleotide sequence encoding a signal peptide directing the cell wall degrading enzyme encoded by the DNA to a plastid or apoplast of the transgenic plant; and at least one DNA comprising a flowering locus c gene coding region operably linked to a constitutive promoter, wherein the transgenic plant expresses the one or more cell wall degrading enzymes and a transcription factor encoded by the flowering locus c gene that delays flowering while increasing biomass and enabling isolation of increased amounts of the hydrolyzing enzyme from the transgenic plant as compared to a non-transgenic plant from which the transgenic plant is derived;
(b) growing the transgenic plant for a time to accumulate the one or more cell wall degrading enzymes;
(c) harvesting the transgenic plant which has accumulated the one or more cell wall degrading enzymes; and
(d) grinding the transgenic plant to provide an enzyme extract comprising the one or more cell wall degrading enzymes that accumulated in the transgenic plant.

35. A method for converting lignocellulosic material to fermentable sugars comprising:
(a) providing a transgenic plant capable of expressing one or more cell wall degrading enzymes comprising at least one DNA comprising a cell wall degrading enzyme coding region operably linked to a nucleotide sequence encoding a signal peptide directing the cell wall degrading enzyme encoded by the DNA to a plastid or apoplast of the transgenic plant; and at least one DNA comprising a flowering locus c gene coding region operably linked to a constitutive promoter, wherein the transgenic plant expresses the one or more cell wall degrading enzymes and a transcription factor encoded by the flowering locus c gene that delays flowering while increasing biomass and enabling isolation of increased amounts of the hydrolyzing enzyme from the transgenic plant as compared to a non-transgenic plant from which the transgenic plant is derived;
(b) growing the transgenic plant for a time sufficient for the transgenic plant to accumulate the one or more cell wall degrading enzymes;
(c) harvesting the transgenic plant which has accumulated the one or more cell wall degrading enzymes;
(d) grinding the transgenic plant to provide an enzyme extract comprising the one or more cell wall degrading enzymes that accumulated in the transgenic plant;
(e) incubating the lignocellulosic material in the enzyme extract to produce the fermentable sugars from the lignocellulose in the plant material; and
(f) extracting the fermentable sugars produced from the lignocellulosic material.


SEQUENCE LISTING (35 sequences)

SEQ ID NO: 1 - DNA, 1110 bp, Oryza sativa
gggtcggagatgccaccacggccacaacccacgagcccggcgcgacaccaccgcgcgcgttgagccagccacaaacgcccgcggataggcgcgccgcacgcggccaatcctaccacatccccggcctccgcggctcgagcgccgtgccatccgatccgctgagttttggctatttatacgtaccgcgggagcctgtgtgcagagagtgcatctcaagaagtactcgagcaaagaaggagagagcttggtgagctgcagagatggccccctccgtgatggcgtcgtcggccaccaccgtcgctcccttccagggctcaagtccaccgccggcatgccgtcgcccgccgtccgaactccagcttcggcaacgtcagcatggcggcaggatcaggtgcatgcaggtaattacctactgatccaacacacattcttcttcttcttcttcttcttaaccaacattaaccaacaactcaattatcgtttattcattgaggtgtggccgattgagggcatcaagaagttcgagaccctctcctacctgccaccgctcaccgtggaggacctcctgaagcagatcgagtacctagctccgttccaagtggtgccctgcctcgagttcagcaaggtcggatttgtctaccgtgagaaccacaagtcccctggatactacgacggcaggtactggaccatgtggaagctgcccatgttcgggtgcaccgacgccacccaggtcgtcaaggagctcgaggaggccaagaaggcgtaccctgatgcattcgtccgtatcatcggcttcgacaacgttaggcaggtgcagctcatcagcttcatcgcctacaacccgggctgcgaggagtctggtggcaactaagccgtcatcgtcatatatagcctcgtttaattgttcatctctgattcgatgatgtctcccaccttgtttcgtgtgttcccagtttgtttcatcgtcttttgattttaccggccgtgctctgcttttgttttttcttttcacctgattctctctctgacttgatgtaagagtggtatctgctacgactatatgttgtttgggtgaggcatatgtgaatgaaatctatgaaagctccggct

SEQ ID NO: 2 - protein, 38 aa, Oryza sativa
MAPSVMASSATTVAPFQGSSPPPACRRPPSELQLRQRQ

SEQ ID NO: 3 - protein, 6 aa, Artificial Sequence
  Signal peptide targets the peroxisomes of plants
RAVARL

SEQ ID NO: 4 - DNA, 3004 bp, Acidothermus cellulolyticus
  CDS (824)..(2512): E I beta-1,4-endoglucanase precursor
ggatccacgttgtacaaggtcacctgtccgtcgttctggtagagcggcgggatggtcacccgcacgatctctcctttgttgatgtcgacggtcacgtggttacggtttgcctcggccgcgattttcgcgctcgggcttgctccggctgtcgggttcggtttggcgtggtgtgcggagcacgccgaggcgatcccaatgagggcaagggcaagagcggagccgatggcacgtcgggtggccgatggggtacgccgatggggcgtggcgtccccgccgcggacagaaccggatgcggaataggtcacggtgcgacatgttgccgtaccgcggacccggatgacaagggtgggtgcgcgggtcgcctgtgagctgccggctggcgtctggatcatgggaacgatcccaccattccccgcaatcgacgcgatcgggagcagggcggcgcgagccggaccgtgtggtcgagccggacgattcgcccatacggtgctgcaatgcccagcgccatgttgtcaatccgccaaatgcagcaatgcacacatggacagggattgtgactctgagtaatgattggattgccttcttgccgcctacgcgttacgcagagtaggcgactgtatgcggtaggttggcgctccagccgtgggctggacatgcctgctgcgaactcttgacacgtctggttgaacgcgcaatactcccaacaccgatgggatcgttcccataagtttccgtctcacaacagaatcggtgcgccctcatgatcaacgtgaaaggagtacgggggagaacagacgggggagaaaccaacgggggattggcggtgccgcgcgcattgcggcgagtgcctggctcgcgggtgatgctgcgggtcggcgtcgtcgtcgcggtgctggcattggttgccgcactcgccaacctagccgtgccgcggccggctcgcgccgcgggcggcggctattggcacacgagcggccgggagatcctggacgcgaacaacgtgccggtacggatcgccggcatcaactggtttgggttcgaaacctgcaattacgtcgtgcacggtctctggtcacgcgactaccgcagcatgctcgaccagataaagtcgctcggctacaacacaatccggctgccgtactctgacgacattctcaagccgggcaccatgccgaacagcatcaatttttaccagatgaatcaggacctgcagggtctgacgtccttgcaggtcatggacaaaatcgtcgcgtacgccggtcagatcggcctgcgcatcattcttgaccgccaccgaccggattgcagcgggcagtcggcgctgtggtacacgagcagcgtctcggaggctacgtggatttccgacctgcaagcgctggcgcagcgctacaagggaaacccgacggtcgtcggctttgacttgcacaacgagccgcatgacccggcctgctggggctgcggcgatccgagcatcgactggcgattggccgccgagcgggccggaaacgccgtgctctcggtgaatccgaacctgctcattttcgtcgaaggtgtgcagagctacaacggagactcctactggtggggcggcaacctgcaaggagccggccagtacccggtcgtgctgaacgtgccgaaccgcctggtgtactcggcgcacgactacgcgacgagcgtctacccgcagacgtggttcagcgatccgaccttccccaacaacatgcccggcatctggaacaagaactggggatacctcttcaatcagaacattgcaccggtatggctgggcgaattcggtacgacactgcaatccacgaccgaccagacgtggctgaagacgctcgtccagtacctacggccgaccgcgcaatacggtgcggacagcttccagtggaccttctggtcctggaaccccgattccggcgacacaggaggaattctcaaggatgactggcagacggtcgacacagtaaaagacggctatctcgcgccgatcaagtcgtcgattttcgatcctgtcggcgcgtctgcatcgcctagcagtcaaccgtccccgtcggtgtcgccgtctccgtcgccgagcccgtcggcgagtcggacgccgacgcctactccgacgccgacagccagcccgacgccaacgctgacccctactgctacgcccacgcccacggcaagcccgacgccgtcaccgacggcagcctccggagcccgctgcaccgcgagttaccaggtcaacagcgattggggcaatggcttcacggtaacggtggccgtgacaaattccggatccgtcgcgaccaagacatggacggtcagttggacattcggcggaaatcagacgattaccaattcgtggaatgcagcggtcacgcagaacggtcagtcggtaacggctcggaatatgagttataacaacgtgattcagcctggtcagaacaccacgttcggattccaggcgagctataccggaagcaacgcggcaccgacagtcgcctgcgcagcaagttaatacgtcggggagccgacgggagggtccggaccgtcggttccccggcttccacctatggagcgaacccaacaatccggacggaactgcaggtaccagagaggaacgacacgaatgcccgccatctcaaaacggctgcgagccggcgtcctcgccggggcggtgagcatcgcagcctccatcgtgccgctggcgatgcagcatcctgccatcgccgcgacgcacgtcgacaatccctatgcgggagcgaccttcttcgtcaacccgtactgggcgcaagaagtacagagcgaacggcgaaccagaccaatgccactctcgcagcgaaaatgcgcgtcgtttccacatattcgacggccgtctggatggaccgcatcgctgcgatcaacggcgtcaacggcggacccggcttgacgacatatctggacgccgccctctcccagcagcagggaaccacccctgaagtcattgagattgtcatctacgatctgccgg

SEQ ID NO: 5 - protein, 562 aa, Acidothermus cellulolyticus
VPRALRRVPGSRVMLRVGVVVAVLALVAALANLAVPRPARAAGGGYWHTSGREILDANNVPVRIAGINWFGFETCNYVVHGLWSRDYRSMLDQIKSLGYNTIRLPYSDDILKPGTMPNSINFYQMNQDLQGLTSLQVMDKIVAYAGQIGLRIILDRHRPDCSGQSALWYTSSVSEATWISDLQALAQRYKGNPTVVGFDLHNEPHDPACWGCGDPSIDWRLAAERAGNAVLSVNPNLLIFVEGVQSYNGDSYWWGGNLQGAGQYPVVLNVPNRLVYSAHDYATSVYPQTWFSDPTFPNNMPGIWNKNWGYLFNQNIAPVWLGEFGTTLQSTTDQTWLKTLVQYLRPTAQYGADSFQWTFWSWNPDSGDTGGILKDDWQTVDTVKDGYLAPIKSSIFDPVGASASPSSQPSPSVSPSPSPSPSASRTPTPTPTPTASPTPTLTPTATPTPTASPTPSPTAASGARCTASYQVNSDWGNGFTVTVAVTNSGSVATKTWTVSWTFGGNQTITNSWNAAVTQNGQSVTARNMSYNNVIQPGQNTTFGFQASYTGSNAAPTVACAAS

SEQ ID NO: 6 - DNA, 1467 bp, Actinomyces naeslundii
  CDS (1)..(1467): beta-glucosidase
  misc_feature (339)..(339): nucleotide is uncertain
  misc_feature (443)..(443): nucleotide is uncertain
  misc_feature (947)..(947): nucleotide is uncertain
atgaccgccacgtccactacttctaagagcaatccgaacttccccgacggcttcctgtggggcggggccaccgccgccaaccagatcgagggcgcttacaacgaggacggcaagggcctgtccgtccaggacgtcatgcctcggggcatcatggcccaccccacccaggctcccacaccggataaccttcaagctcgaggcgatcgaccttctaccaccgcttacgccgaggacatctccctgttcgcggagatgggtttcaaggtcttccgcttctccatcgcctggagccgcatcttcccgctcggcgacgagaccgagcccaatgaggaaggactngccttctacgaccgggtcctcgacgagctcgagaagcacgggatcgagccactggtcaccatcagccactacgagaccccgctgcacctggcgcgcacctacgncggctggaccgaccgccgcctcatcggcttcttcgagcgctacgcccgcaccctgttcgagcgctatggcaagcgggtcaagtactggctcaccttcaacgagatcaactccgtgctccatgagcccttcctatctgggggcgtcgccacgcccaaggacaggccccccgagcaggacctctaccaggccatccaaaacgagctcgtcgcctccgcggccgcgaccaggatcgcccatgagaccaaccccgacatccaggtcggctgcatgatcctggccgatcccacctacccgctcacccctgatccccgggacgtgtgggcggccaagcaggcagagcgcgccaactacgccttcggagacctccacgtacgtggtgagtaccccggatacctgcggcggaccctgcgggacaagggcatcgagctggagatcaccgaggaggaccgcgtgctgctgcgggagcacaccgtcgacttcgtctccttctcctactacatgtncgtgtgcgagaccgtcacccagtcggccgaggccggccggggcaacctcatgggcggcgtccccaatcccaccctcgaggcctccgagtggggatggcagatcgacccggcgggcctgcgcaccatcctgaacgactactgggaccgctggggcaagcctctgttcatcgtcgagaacggcctgggagccaaggacgtcctcgttgacggacccaacggtcccacggtcgaggacgactaccgcatcgcctacatgaacgaccacctggtccaggtcgccgaggccattgccgacggcgtcgaggtcctgggctacacctcctggggctgcatcgacctggtctcggcctccaccgcccagatgtccaagcgctacgggttcatctacgtggaccgtgacgacggcggcaacggcaccctggcccgctaccgcaagaagtccttcggctggtaccgcgacgtcatcgcctccaacggtgcctccctcgtgcctccggtgcaggaaccgccgcgggggtag

SEQ ID NO: 7 - protein, 488 aa, Actinomyces naeslundii
  misc_feature (113)..(113): The ′Xaa′ at location 113 stands for Leu.
  misc_feature (148)..(148): The ′Xaa′ at location 148 stands for Asp, Gly, Ala, or Val.
  misc_feature (316)..(316): The ′Xaa′ at location 316 stands for Tyr, Cys, Ser, or Phe.
MTATSTTSKSNPNFPDGFLWGGATAANQIEGAYNEDGKGLSVQDVMPRGIMAHPTQAPTPDNLQARGDRPSTTAYAEDISLFAEMGFKVFRFSIAWSRIFPLGDETEPNEEGXAFYDRVLDELEKHGIEPLVTISHYETPLHLARTYXGWTDRRLIGFFERYARTLFERYGKRVKYWLTFNEINSVLHEPFLSGGVATPKDRPPEQDLYQAIQNELVASAAATRIAHETNPDIQVGCMILADPTYPLTPDPRDVWAAKQAERANYAFGDLHVRGEYPGYLRRTLRDKGIELEITEEDRVLLREHTVDFVSFSYYMXVCETVTQSAEAGRGNLMGGVPNPTLEASEWGWQIDPAGLRTILNDYWDRWGKPLFIVENGLGAKDVLVDGPNGPTVEDDYRIAYMNDHLVQVAEAIADGVEVLGYTSWGCIDLVSASTAQMSKRYGFIYVDRDDGGNGTLARYRKKSFGWYRDVIASNGASLVPPVQEPPRG

SEQ ID NO: 8 - DNA, 3072 bp, Streptococcus salivarius
  CDS (392)..(2860): 1,6-alpha-glucanhydrolase
aactgaggccgttgctccagtagcgacaacagaaataggtccatcaactgctactgttgcgacagatactgcaacaacagcgacagcttctacaatcttttcacaagctgtgccagcagaaagtgctagctcagaaacgcttgtagccagtgaagcactagctcctgagtcagctgctgtggaaaccatcacatcatcatctgataatgctactgaagcaggacgccattcaactgctcaagtaacaccagttacagaagtgacagagcaaaacttgaatggtgatgcctacttgacagatccagaaacaacaaaagcagcttatagcaagacagatggtgatattaattattccgttgttgtgtctaatccaacagcagaaactaagacgatgactgtcaacttgacacttcaacatgcttcagaaattatcggtcaagataacgttgaccttacgctagcggcaggagcttcagccaaggtttcaaacttgacagtagcgtcagagtggttgacaaacaatacaggttacttggtgacaatcagtgtcaacgataaatcaggcaatgtcttgtcaagcaagcgcgctggcttgtctgttgaagatgattggacagttttcccacgttacggtatcgtagcaggttcaccaactgatcaaaacagtattcttgttaaaaatcttgaagcctaccgtaaagagcttgagctcatgaagtctatgaatatcaactcatatttcttctatgatgcttataatgaagctacagatcctttcccagaaggtgtcgatagctttgttcaaaaatggaatacctggagtcacactcaggttgacactaaggctgttaaagaattggttgatcaagttcataagtcaggtgctgttgccatgctttataacatgatttcagcagattcaaatccaaagaatccggcccttccacttgctgctttggcttataacttctacgatagctttggtaagaagggtgaaccgatgacttacactatcggtgataacccaactcaagtttactatgatccggcgaatccagattggcaaaaatacatcgcaggtgtcatgaaatcagctatggatcgtatgggattcgatggttggcaaggtgatacaattggtgacaaccgtgtgactgattatgagcaccgtaacagcacagacgaggctgactcacacatgatgtctgattcatatgcgtcatttattaatgccatgaaggacctcatcggtgaaaagtactacatcacaatcaatgatgttaatggtggtaatgatgataaactagccaaggcacgtcaagatgttgtttataatgagctttggacaaacggtggttcagttattccaggacgtatgcaggttgcctatggtgatttgaaagcacgtatcgatatggtacgcaataaaactggtaaatcacttatcgttggtgcctacatggaagaaccagggattgattatactgttcctggcggaaaagcaactaacggtgctggtaaagatgcccttgctggtaaaccattgcaagctgatgcgactcttctcgtagatgcgacagtagctgcagcaggtggttatcacatgtccattgcagcccttgcaaatgctaatgcggcccttaacgtccttcaaagtgcctattacccaacgcaatacctcagtgtggctaaagacactattcgtaagctttacaattaccaacagtttatcactgcttatgaaaatcttctccgcggtgagggtgtgacaaacagcactcaggctgtatctacaaagaatgcttctggtgaaatcctttctaaagatgctcttggtgtgacaggagatcaagtttggacatttgctaaatcaggaaaaggtttctcaactgttcaaatgattaatatgatgggcatcaatgcgggctggcataatgaagagggttatgcggacaataaaacaccggacgcacaagaaaatctcacagttcgtcttagcctagcaggtaaaacagcccaagaagcagctaaaattgctgatcaagtctatgtgacgtcaccggatgattgggcaacttcaagcatgaagaaggcacaagcaagccttgaaacagatgaaaatggtcaaccagtgcttgtcatttcagttcctaaactaactctttggaacatgctttatatcaaggaagacacaacagcaacaccggtagaaccagttactaaccaagctggtaagaaagtagataataccgtaacatctgaagcaagctcagaaacagctaaatcagaaaatacaacagtaaataaaggttcagaggctccaactgatacgaaaccatctgttgaagctcctaaactagatgaaacaactaaaccagcaccatcagttgacgagttagtaaactcagcagctgttccagtggcgatagctgtgtcagagaccgcacatgataagaaagatgacaactcagtatctaatacggatcaaggtacagtagcatcagattcaatcactacaccagcttcagaggctgcaagcacagctgcctcaacagtctcatcagaagtatcagaaagtgtaacagtatcatcggaaccatcagaaactgaaaatagttcagaagcatcaacttcagagtcagcaactccaacgacgacagcaatttcagaatcacatgcagtagttgaaccagtggcttctttgacagaatcagagagtcaggcaagcactagccttgtttcagaaactacaagcacaattgtctcagttgctccgtcagaagtatcagaaagcacatcagaggaagtcatccttatggactatcagaaaacatcaatagttggaatagactctctgtagctcctcgcgtctcagaaaccttaccaagtacttctgaaacgattacagaagcagcatcactctttagcaactatgcaagatattcagaaacagcaagctcagaatctcactctatggtagcagcttcttcagaagtttctattgaaaaattagcagtatctatcttgaaagatactgagggaggcttgtatgatgcaacaacaatcagaaat

SEQ ID NO: 9 - protein, 822 aa, Streptococcus salivarius
MTVNLTLQHASEIIGQDNVDLTLAAGASAKVSNLTVASEWLTNNTGYLVTISVNDKSGNVLSSKRAGLSVEDDWTVFPRYGIVAGSPTDQNSILVKNLEAYRKELELMKSMNINSYFFYDAYNEATDPFPEGVDSFVQKWNTWSHTQVDTKAVKELVDQVHKSGAVAMLYNMISADSNPKNPALPLAALAYNFYDSFGKKGEPMTYTIGDNPTQVYYDPANPDWQKYIAGVMKSAMDRMGFDGWQGDTIGDNRVTDYEHRNSTDEADSHMMSDSYASFINAMKDLIGEKYYITINDVNGGNDDKLAKARQDVVYNELWTNGGSVIPGRMQVAYGDLKARIDMVRNKTGKSLIVGAYMEEPGIDYTVPGGKATNGAGKDALAGKPLQADATLLVDATVAAAGGYHMSIAALANANAALNVLQSAYYPTQYLSVAKDTIRKLYNYQQFITAYENLLRGEGVTNSTQAVSTKNASGEILSKDALGVTGDQVWTFAKSGKGFSTVQMINMMGINAGWHNEEGYADNKTPDAQENLTVRLSLAGKTAQEAAKIADQVYVTSPDDWATSSMKKAQASLETDENGQPVLVISVPKLTLWNMLYIKEDTTATPVEPVTNQAGKKVDNTVTSEASSETAKSENTTVNKGSEAPTDTKPSVEAPKLDETTKPAPSVDELVNSAAVPVAIAVSETAHDKKDDNSVSNTDQGTVASDSITTPASEAASTAASTVSSEVSESVTVSSEPSETENSSEASTSESATPTTTAISESHAVVEPVASLTESESQASTSLVSETTSTIVSVAPSEVSESTSEEVILMDYQKTSIVGIDSL

SEQ ID NO: 10 - DNA, 2220 bp, Trichoderma longibrachiatum
aaggttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactggcatcatgtatcggaagttggccgtcatcacggccttcttggccacagctcgtgctcagtcggcctgcactctccaatcggagactcacccgcctctgacatggcagaaatgctcgtctggtggcacttgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgtatcttcttgtgggctcccagctgactggccaatttaaggtgcggcttgaacggagctctctacttcgtgtccatggacgcggatggtggcgtgagcaagtatcccaccaacaacgctggcgccaagtacggcacggggtactgtgacagccagtgtccccgcgatctgaagttcatcaatggccaggccaacgttgagggctgggagccgtcatccaacaacgcaaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggagacagaattcggcggatctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaaccgtggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgtaaagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtacca

SEQ ID NO: 11 - DNA, 1263 bp, Phanerochaete chrysosporium
  CDS (34)..(1152): ckg4 ligninase precursor
gctacagctcaccgtccggtctcagcagcagcaatggcgttcaagcagctcctcgcagccctctccgtcgccctgaccctccaggtcacccaagctgccccgaacctcgacaagcgcgtcgcttgccccgacggcgtgcacaccgcctccaacgcggcgtgctgtgcatggttcccggtcctcgatgatatccagcagaacctcttccacggtggccagtgcggtgccgaggcccacgaggcccttcgtatggtcttccacgactccatcgctatctcgcccaagcttcagtcgcagggcaagtttggcggcggcggcgcggacggctcgatcattaccttctcctcgatcgagaccacgtaccacccgaacatcggcctcgacgaggtcgtcgccatccagaagccgttcatcgcgaagcacggcgtcacccgtggcgacttcatcgcattcgctggtgccgtcggcgtgagcaactgcccgggcgcgccgcagatgcagttcttccttggccgccccgaggcaacgcaggccgcccccgacggtctcgtgcccgagcccttccacaccatcgatcaggttctcgctcgcatgcttgacgctggtggcttcgacgagatcgagactgtctggctgctctctgcccactccatcgcggctgcgaacgacgtcgacccgaccatctccggcctgccgttcgactccactcccggccagttcgactcccagttcttcgtcgagacgcagctccgcggtaccgcattccctggcaagactggtatccagggcaccgtcatgtccccgctcaagggcgagatgcgtctgcagacggaccacttgttcgcgcgtgactcgcgcacggcatgcgagtggcagtccttcgtcaacaaccagacgaagctgcaggaggacttccagttcatcttcacggcgctctcgacgctcggccacgacatgaacgccatgatcgactgctccgaggtcatccccgcgcccaagcccgtcaacttcggcccgtcgttcttccccgccggtaagacgcacgccgacatcgagcaggcctgcgcatccacgccgttcccgacgctcatcaccgcccccggtccctctgcgtccgtcgctcgcatccccccgccgccgtcccccaactaagctatgtctatgctggacatgctctcggttctacctcgtcggtatcgtcgcacggttatctcgcgtttgcatcatgtatacctgctcgtggaatatacaaagtggtctatc

SEQ ID NO: 12 - protein, 372 aa, Phanerochaete chrysosporium
MAFKQLLAALSVALTLQVTQAAPNLDKRVACPDGVHTASNAACCAWFPVLDDIQQNLFHGGQCGAEAHEALRMVFHDSIAISPKLQSQGKFGGGGADGSIITFSSIETTYHPNIGLDEVVAIQKPFIAKHGVTRGDFIAFAGAVGVSNCPGAPQMQFFLGRPEATQAAPDGLVPEPFHTIDQVLARMLDAGGFDEIETVWLLSAHSIAAANDVDPTISGLPFDSTPGQFDSQFFVETQLRGTAFPGKTGIQGTVMSPLKGEMRLQTDHLFARDSRTACEWQSFVNNQTKLQEDFQFIFTALSTLGHDMNAMIDCSEVIPAPKPVNFGPSFFPAGKTHADIEQACASTPFPTLITAPGPSASVARIPPPPSPN

SEQ ID NO: 13 - DNA, 1285 bp, Phanerochaete chrysosporium
  CDS (34)..(1149): CKG5 ligninase precursor
gtcagactctccaacggttgcctttggacagacatggccttcaagaagctccttgctgttcttaccgccgctctctccctccgcgctgcgcagggtgcggccgtcgagaagcgcgcgacctgctcgaacggcaaggtcgtccccgcggcgtcttgctgcacctggttcaacgttctgtccgatatccaggagaacctcttcaatggcggccagtgtggcgccgaggctcatgagtcgatccgtctcgtcttccacgacgccatcgctatctctcccgctatggagccgcaggccagttcggtgcgaggcgccgatggttctatcatgatcttcgacgagatcgagaccaacttccatcccaacatcggtctcgacgagatcgtccgcctgcagaagccgttcgtccagaagcacggtgtcactcccggtgacttcatcgccttcgctggcgcggtggcgctcagtaactgccccggtgctccgcagatgaacttcttcactggtcgtgctccggcaactcagccagcccctgacggcctcgtcccagagcccttccactctgttgaccaaatcatcgaccgtgtcttcgatgccggtgaattcgatgagctcgagctcgtctggatgctctctgcacactccgtcgcggctgccaacgatatcgacccgaacatccagggcttgcccttcgactcgacccccggtattttcgattcccagttcttcgtcgagactcagcttgctggcaccggcttcactggcggttctaacaaccagggcgaggtttcctccccgcttccaggcgagatgcgtctccagtctgacttcctgatcgctcgtgacgcgcgcaccgcctgcgagtggcagtcgttcgtcaacaaccagtccaagctcgtctccgacttccaattcatcttcctcgccctcactcagctcggccaggacccggatgcgatgaccgactgctctgctgtcatccccatctccaagcccgccccgaacaacacccccggattctccttcttcccgcccggcatgacgatggacgatgtcgagcaggcttgcgccgagacgcccttcccgactctctcgactctccctggccccgcgacctccgtcgctcgcatccctcctcctcctggtgcttaagcagccatcagacttcggatcacaccccggtattggcaacggaaatttagaacgaagatcgtccagtgttttgaagtagaaatgtgcttgtactgtgtaaacagctcttttgacgaaatacactctgatttcgtcg

SEQ ID NO: 14 - protein, 371 aa, Phanerochaete chrysosporium
MAFKKLLAVLTAALSLRAAQGAAVEKRATCSNGKVVPAASCCTWFNVLSDIQENLFNGGQCGAEAHESIRLVFHDAIAISPAMEPQASSVRGADGSIMIFDEIETNFHPNIGLDEIVRLQKPFVQKHGVTPGDFIAFAGAVALSNCPGAPQMNFFTGRAPATQPAPDGLVPEPFHSVDQIIDRVFDAGEFDELELVWMLSAHSVAAANDIDPNIQGLPFDSTPGIFDSQFFVETQLAGTGFTGGSNNQGEVSSPLPGEMRLQSDFLIARDARTACEWQSFVNNQSKLVSDFQFIFLALTQLGQDPDAMTDCSAVIPISKPAPNNTPGFSFFPPGMTMDDVEQACAETPFPTLSTLPGPATSVARIPPPPGA

SEQ ID NO: 15 - DNA, 360 bp, Solanum tuberosum
tgaccctagacttgtccatcttctggattggccaagttaattaatgtatgaaataaaaggatgcacacatagtgacatgctaatcactataatgtgggcatcaaagttgtgtgttatgtgtaataactaattatctgaataagagaaagagagatcatccatatttcttatcctaaatgaatgacagtgtctttataattctttgatgaacagatgcattttattaaccaattccatatacatataaatattaatcatatataattaatatcaattggttagcaaaacccaaatctagtctaggtgtgttttgctaattatgggggatagagcaaaaaagaaactaacgtctcaagaatc

SEQ ID NO: 16 - DNA, 2521 bp, Agrobacterium tumefaciens
  CDS (585)..(1826): nopaline synthetase
tagccgacccagacgagccaagggatctttttggaatgctgctccgtcgtcaggctttccgacgtttgggtggttgaacagaagtcattatcgtacggaatgccaagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttatccgcaacttctttacctatttccgccgcagatcaccatccgcttcccttgaccgtaggtgtcctcggttctggtcacgcggggactgcattagcggcttggttcgcctcccggcatgttcccacggcgctgtgggcaccagcagatcatccaggatcgatctcagcaatcaaggccaatgaaggagttatcaccaccgagggaatgattaacggtccatttagggtctcagcctgtgatgaccttgccgcagttattcgctccagccgtgtactgattattgtaacccgtgcggacgttcacgacagcttcgtcaacgaactcgccaacttcaacggcgaactcgcaacaaaggatattgtcgtcgtgtgcggccatggcttctccatcaagtacgagagacagctgcgattcaagcgaatattcgagacggataattcgcccataacgtctaagctatcggatcaaaaaaaatgtaacgtcaacatcaaggaaatgaaagcgtctttcggactgtcatgtttcccaattcatcgcgatgatgctggcgtgattgatctacccgaagataccaagaacatctttgcccagctattttccgctagaatcatctgcatcccgccgttgcaagtgctattcttttccaactgtatcactcatgcggttccggcagtcatgaacatcggaagactccgcgacccagccaattctcttactaaaagagctgagaagtggcttcttgaactagacgagcgaaccccacgagccgagaagggctttttcttttatggtgaaggatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcaaacttcggtactgtgtaatgacgatgagcaatcgagaggctgactaacaaaaggtatgcccaaaaacaacctctccaaactgtttcgaattggaagtttctgctcatgccgacaggcataacttagatattcgcgggctattcccactaattcgtcctgctggtttgcgccaagataaatcagtgcatctccttacaagttcctctgtcttgtgaaatgaactgctgactgccccccaagaaagcctcctcatctcccagttggcggcggctgatacaccatcgaaaacccacgtccgaacacttgatacatgtgcctgagaaataggcctacgtccaagagcaagtcctttctgtgctcgtcggaaattcctctcctgtcagacggtcgtgcgcatgtcttgcgttgatgaagctt

SEQ ID NO: 17 - protein, 413 aa, Agrobacterium tumefaciens
MAITLSATSLPISAADHHPLPLTVGVLGSGHAGTALAAWFASRHVPTALWAPADHPGSISAIKANEGVITTEGMINGPFRVSACDDLAAVIRSSRVLIIVTRADVHDSFVNELANFNGELATKDIVVVCGHGFSIKYERQLRFKRIFETDNSPITSKLSDQKKCNVNIKEMKASFGLSCFPIHRDDAGVIDLPEDTKNIFAQLFSARIICIPPLQVLFFSNCITHAVPAVMNIGRLRDPANSLTKRAEKWLLELDERTPRAEKGFFFYGEGSNTYVCNVQEQIDHERRKVAAACGLRLNSLLQECNDEYDTDYETLREYCLAPSPHNVHHACPDNMEHRYFSEELCSLEDVAAIAAIANIELPLTHAFINIIHAGKGKINPTGKSSSVIGNFSSSDLIRFGATHVFNKDEMVE

SEQ ID NO: 18 - DNA, 835 bp, Streptomyces hygroscopicus
gctcgctgtcattttcgagacgccatctttggaagcggtggccgaatccgtactgcgcggactcgacgacgcgtaaaacgatcgaccacgtacacgagtccggacacggggcgaggaggcccggttccggcaccgaggaagaccgaaggaagaccacacgtgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgaacggagtgcgcgtgggcatcgcccgagttggagctggtacgggaactcatcgaactcaactggcatacccgcaatggtgaggtggaaccgcggcggatcgcgtacgaccgtgcccaggaggcct

SEQ ID NO: 19 - DNA, 623 bp, Oryza sativa
tatatacatacccccccctctcctcccatccccccaaccctaccaccaccaccaccaccacctcctcccccctcgctgccggacgacgagctcctcccccctccccctccgccgccgccggtaaccaccccgcgtccctctcctctttctttctccgttttttttttccgtctcgtctcgatctttggccttggtagtttgggggcgagaggcggcttcgtcgcccagatcggtgcgcgggaggggcgggatctcgcggctgggtctcggcgtgcggccggatcctcgcggggaatggggctctcggatgtagatctgatccgccgttgttgggggagatgatggggcgtttaaaatttcgccatgctaaacaagatcaggaagaggggaaaagggcactatggtttatatttttatatatttctgctgctgctcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagatggctgacgccgaggatgggggatccccgggtggtcagtcccttatg

SEQ ID NO: 20 - DNA, 1506 bp, Trichoderma longibrachiatum
accatggctcaatctgcttgtactcttcaatctgagactcatcctccacttacttggcagaagtgttcatctggtggtacttgtactcaacagactggatctgttgttattgatgctaactggagatggactcatgctactaactcttctactaactgctatgatggtaacacttggtcatctactctttgtcctgataacgagacttgtgctaagaactgctgtcttgatggtgctgcttacgcttctacttacggagttactacttctggaaactctctttctattggattcgttactcagtctgctcagaagaacgttggtgctaggttgtacttgatggcttctgatactacttaccaagagttcactcttcttggtaacgagttctctttcgatgttgatgtttctcaacttccatgtggtttgaacggagctttgtacttcgtttctatggatgctgatggtggagtttctaagtatccaactaacactgctggagctaagtatggtactggttactgtgattctcagtgtccaagagatcttaagttcattaacggacaagctaatgttgagggatgggagccatcttctaacaatgctaacactggtattggaggtcatggatcttgttgctctgagatggatatttgggaggctaactctatctctgaggctcttactccacatccatgcactactgttggacaagagatttgcgagggagatggttgtggtggaacttactctgataacagatacggaggtacttgcgatccagatggatgtgattggaatccatacagacttggtaacacttctttctacggtccaggatcttcattcactcttgatactactaagaagttgactgttgttactcagttcgagacttctggtgctatcaacagatactacgttcagaatggagttactttccaacaacctaacgctgagcttggttcttactctggtaacgagttgaacgatgattactgtactgctgaggaagctgagtttggtggatcatctttctctgataagggtggacttactcagttcaagaaggctacttctggtggtatggttcttgttatgtctctttgggatgattactacgctaacatgttgtggcttgattctacttacccaactaacgagacttcttctactccaggtgctgttagaggatcttgctctacttcttctggtgttcctgctcaagttgaatctcaatctcctaatgctaaggttactttctctaacatcaagttcggaccaattggatctactggtaacccttctggaggtaatccacctggaggtaatccacctggaactactacaactaggagaccagctactacaactggatcatctccaggacctactcaatctcattacggtcaatgtggaggtattggttactctggtccaactgtttgtgcttctggaactacttgtcaagttcttaacccttactattctcaatgcctttaatga

SEQ ID NO: 21 - DNA, 26 bp, Artificial Sequence
  PCR primer SP1F
ccgcctaggcgcatggccccctccgt

SEQ ID NO: 22 - DNA, 25 bp, Artificial Sequence
  PCR primer SP3R
cgctgtacacgcacctgatcctgcc

SEQ ID NO: 23 - DNA, 3124 bp, Butyrivibrio fibrisolvens
aacaaacagaattgttggaaacttttttatatgcaatattactttaacacagaatgtctggtgatggtatatgttttttcttcctgagttatatatttcaaatcttagatgtgctatgcttttatggcagaaacatgttatatacaaggtaaagataagtaggataacgaggtatttataatggagaaatgggcaagaatcaaatatacaccaaatcttccgcttggagagaatggtgaaagggttacagcgagtcagaagcacattgagctttcatgcgaggcagcatgtgagggaatggtacttctcaagaatgacagaaacgttcttcctatcagaaagggcacaagagtagccctctttggaaagggagtatttgactatgtaaaaggcggcggtggtagcggagatgtaacagttccttacatcagaaacctctacgaaggcctttctcagtacacatcagacatttcaatttacgacaaatctgtcagattctatcaggaatatgtagcagaccagtacagacttggaattgcaccaggcatgatcaaagagccggctcttccggaagatattcttgcagatgcagcagcctatgcagatactgcaatcatcgcaatcagcagattctccggagaaggctgggacagaaaggttgcaggcgttgacagagaaatcaagtgcgaagccaaggacctcgtagagcagggcaacaagatatttgatcatggtgatttctacctcacaaatgctgagaagaagatggtcaagatggtaaaagagaacttctcaagcgtcattgtagtcatgaatgtcggaggagtcgtagacacaacatggtttaaaaaggatgaccagatttcatcagtcctcatggcatggcagggtggaattgaaggcggacttgccgcagccaggatccttcttggcaaggttaatccttcaggtaagctctcagatacattcgcagcaaggcttgaagactatccttcaacagagggcttccacgaagatgatgactacgtggattacacagaagatatctacgttggctatagatatttcgagaccattcccggggcaaaagagaaagttaactacccctttggctatggcctttcctatacaactttcctgcttgaagactataaggcagagccttttgtggcttcagcagcagacgaggtcggtaaatctgatagcgaccttgcagatgcaatcgtagcctcagttacagtcacaaacattggcaagattccgggcaaagaggttgttcagctctactacagcgctcctcagggcaagctcggtaagcctgctaaagtccttggcggctatgccaagacaaggctactgcagccgggagagagccagagagtgacaattgctctttatatggaggatatggcatcttacgacgaccttggcaaggttaaaaaggctgcctggctccttgaaaaaggtgaatatcatttcttccttggaacatcagtaagagacacaaggcttcttgattacacctatgaactttctaagaacataatagttgaacaggtctcaaacaagctcgttccaacatctcttcccaagagaatgcttgctgatggcacatatgaggaacttcctcagacagaacctgtagatacttatgcaacaatcttcccaagacctaagaactggaaagaaacaattgagcacgacgtattaaagactcctgtagttcgtccacaggacagattccagctctttttgccacctaaggaaggtgaccctaagaaatttatcgaagttgcagaatgcaaggtgacacttgaagactttattgcacagctatctaacgagcagcttgcaagccttcttggaggacagccaaatgtcggaatggctaacacctttggatacggcaaccttcctgaggttggagttcctaatgcccagacctgtgatggtcctgcaggtgtccgtattgcaccggaagttggtgttgtgactacagcattcccatgttcaacccttcttgcatgcacatggaatgaagatatctgctacgaagtcggagttgcaggcggagaagaggccaaggagtgcaattttggtgcatggcttactcctgctgttaacatccatagaagccctctttgcggcagaaactttgagtactactccgaagatccattccttgcaggtaaacaggcagcagctatggttcgtggtatccagagcaacaacataattgctacacctaaacattttgccctcaacaacaaggaatccaatagaaaaggcagcgattcacgtgcttctgagcgtgcgatcagagaaatatatttaaaggcctttgaaatcattgttaaagagcagagccctggagcatcatgtcttcaatacaatatagttaacggtcagagatcatccgaatctcacgacctcctcacaggaatcctccgcgatgagtggggctttgaaggtgttgtagtcagcgactggtggggctttggtgagcattacaaggaagtccttgcaggcaacgatatcaagatgggctgtggctatacagaacagctccttgaagcaattgataagaaagctcttaagagaaaagatttggaaaagaggcagagcgagtcctcaagatgcttctcaaactcgactaagctcaaagccgcttagaataaacgtttaagtcataaaaaagtaatgtattgttcataaactatcaatatttttctgacaatttgtcgtataatataaatatgatatagttgttgggaatatcttctgagaattgtatcaggagaaagttgtagaactacaactctcggaggtatgtctatgaacaaaaaagctattgttggtatttttatgtccattttgatggcagggctcgttggatgtgccggtagcagtgatgcccaggcaggagatgacctcaagccggttatttatctttatccacaggaagataataccgagatttcagtaagccttgattataacggaaatctggttgacctgattcctgagtttaatgcagataagacatggaatgttacagctaacaaagatggcaagattacctttgaaggacagacttatgactatctgttttgggaaggcgatc

SEQ ID NO: 24 - DNA, 1640 bp, Cochliobolus carbonum
gatctacaccctagctaatattactctagcagatggaataccgctatcatgacgaggatgtttggcttggctgtgcacaacatccggtcttgtttccactcacatgccatgcgagggcgagtataaagctatcctgagtcggctccatggcacactttcctcaccaaccatcaccacaactaccacttcccttcgcttcattcactcaacaacaacaccatcaaaatggtttctttcacctccatcatcaccgctgctgttgcggctaccggcgctcttgccgcccccgccactgatgtgtctctcgttgcccgtcagaacacccccaacggcgagggtacccacaacggctgcttctggtcttggtggtctgatggcggtgcccgcgctacctacaccaacggtgccggtggtagctacagcgtaagctggggaagcggtggcaacctcgtcggtggaaagggatggaacccaggaactgcccggtatggactgtcttactcaactctgatagactacatacactaacattgatacagtaccatcacctactctggtacttacaactacaacggcaactcctaccttgccgtctacggctggacccgcaacccccttgtcgagtactacgtcgttgagaacttcggcacctacgacccctcttcccagtcccagaacaagggtaccgtcacctctgatggatcttcctacaagatcgctcagtcgacccgtaccaaccagccctccatcgatggcaccaggacctttcagcagtattggtctgttcgtcagaacaagcgctcttccggctccgtcaatatgaagactcactttgacgcctgggccagcaagggcatgaaccttggccagcactactaccagattgttgccaccgagggttacttctccactggtaacgcccagatcaccgtcaactgcccataaattcttcaccacgaagattaaacgaatggccctgatggctctctgaacgactggaagatgacaggcgtggtttttggttacgtgctgtgctagctgagctggtggattcttttctgtatatactatctttgtcaacccgatttgtctatagcttaacaatgtcaaaatcttggcattactcacaacgcgtcttttcatttttgcctctaagtgaattgtgatggtcttgacgcatgatgacgtctactacgagcattctctccggtgtccttctaatgataaaaataaacaaacttcaatactcgcaatatgaaactccgcagtaccaaatttagatgtttgacttcacaataggctggcctatctacgctgcctcatctattacatacaacggcatcgtaccaggagtcatcctcagtgcctcctcaatgattcccaatgcaacatgcaactcctcttccataacagtaatcgggtgtgcaattctgaaaacaccaccaagcgacgccattgtcgccaactgcgcccacaaacccattttctccatcttttgctacacagcagctccgaaatcaggagtcccaaccgtaagaaggtgcagccttcagtgttactggaggcaccgctagtgtggtggtgctttcccaatatagcttagtgcaaatagtaggtgggtagcatctcgctgagagctc

SEQ ID NO: 25 - DNA, 20 bp, Artificial
  Synthetic PCR primer
cgataacctggtcaagatcc

SEQ ID NO: 26 - DNA, 20 bp, Artificial
  synthetic PCR primer
ctgctcccacatgatgatta

SEQ ID NO: 27 - DNA, 17 bp, Artificial
  synthetic PCR primer
gcgggcggcggctattg

SEQ ID NO: 28 - DNA, 20 bp, Artificial
  synthetic PCR primer
gccgacaggatcgaaaatcg

SEQ ID NO: 29 - DNA, 17 bp, Artificial
  synthetic PCR primer
atgagcccagaacgacg

SEQ ID NO: 30 - DNA, 17 bp, Artificial
  synthetic PCR primer
tcagatctcggtgacgg

SEQ ID NO: 31 - DNA, 17 bp, Artificial
  synthetic PCR primer
gcgggcggcggctattg

SEQ ID NO: 32 - DNA, 20 bp, Artificial
  synthetic PCR primer
gccgacaggatcgaaaatcg

SEQ ID NO: 33 - DNA, 1461 bp, Cochliobolus carbonum
cccgggtagagcgatgttcttttaattatgttatgcagatttacgcaagggctacagatctttttgatcaccccgtcgtggccgttgctaccgaaactggatgccgatcggtgcatgtcccctatgtgtctcgacttggtcattgtggccgccgcttagggtgagcttgtcgaagtgagaggccttaacctaccaccaatgtgtacaccatcacgaatagagacagcacgcgagatgagacctcatgaccaccagtgtcatttcatcctgcgattcaggctcaactattactcggcgtagagatataagtcgatagctgctcccagtcagtagcatcactgcatccaacacagatcagcaacactcaacacaagatggtttccttcaagtctctgcttctcgccgctgtggctaccaccagcgtcctcgctgctcccttcgatttccttcgtgagcgcgacgatgtcaacgcgactgctctccttgagaagcgtcagtctactcccagcgccgagggataccacaatggatacttctactcgtggtggactgatggcggtggctctgcccagtacactatgggtgagggcagcaggtactctgtgacctggaggaacactggcaactttgttggtggaaaggggtggaaccctggaagcggccggtaggttgcgaagactgtttggtgatgagaatcttactaatgtggattcgtagtgtcatcaactacggcggagccttcaacccccagggcaacggatacctcgctgtgtacggatggacccgcaacccgcttgtcgagtactacgtgattgaatcctacggaacctacaaccccagcagtggagcccaaatcaagggcagcttccagaccgacggtggtacttacaacgttgccgtctccacccgttacaaccagccctccattgacggaacaaggacctttcagcagtactggtaagtcatttcggtgtttaatcagacagaagcgtagatgttgactgttatgataggtctgtccgcacccagaagcgtgtcggtggaagcgtgaacatgcagaaccacttcaacgcttggtctcgctatggcttgaaccttggtcaacactactaccagatcgtcgccactgagggttaccagtcctctggaagctctgacatctatgtgcagactcagtagagtagttgttgtctgtgaagcgagcaactagtagacgagattagacaagaatagtgtctcggatagttgtaagcaggttggagaaagagcgatgtgttcgatttcctgtacatagaaacatgtcaattcactcgctataaaaccttccgtcctcgaatgtgttctcttttgtattagctgagcattgtatcgtggttcggcaaaagcaggtaaagtttaggtgagggcatttgttataaacagtcatagtctcgtagccatcgaatgctggtgtgtgaattc

SEQ ID NO: 34 - DNA, 1929 bp, Cochliobolus carbonum
ctgcatgactggaccactcagcgcgcggcttccccacaaaagtctatatgaagtatccaccgccgtgatggagcttgattaactgtcgggatggcgacttggcacgcgccttgaaatgcaggatagcatcacgcctaggggcagggccgattgcgctcattggatacgaatacatcaagacactagtgtagaacgattcagacgattcatccatgggcaacgaggggcgagaggtggccccatgacacagggaggaagacagcgggtgaaacaaccgcaatatgtttcctgtggtgtgcacccacgctctttaaccgtctctaagtatataggtcaagacttccaagaccgaataagctcaccaccaagcctttcatcttcaacactcaagcagtctttacttcactcaccctcttttcaattcactcctagcgagaaccagcttcttttcacacaaacacaaacaaatcttcaatctatcaaaatggttgccttcacctccgtcctcctcggcctctccgccattggctctgccttcgccgcccccgttgccgatgtccctgacttcgagttctccggccccaagcacttggctgcccgccaggactacaaccagaactacaagactggtggtaacatccagtacaaccccaccagcaacggttactccgtcaccttctctggcgcccaggacttcgtccttggcaagggctggaagcaaggtactaccaggtaagccacacacagtgttctagacccaaatatcaatcaatactaactcctttccttcaggaccgtcaagtacactggttccacccaggcccaggccggtactgtcctcgtcgctctctacggctggaccaggggcagcaagctcgtcgagtactacatccaggacttcacctctggcggctctggctccgcccagggccagaagatgggccaagtcacctgcgacggctccgtctacgacatctggcagcacacccaggtgaaccagccttccatcgtcggcacgaccaccttcgtccagtacatcagcaaccgcgtcagcaagcgctccaccggcggtaccatcaccaccaagtgccacttcgacgcctgggccaagctcggcatgaaccttggtaaccagtgggactaccagaccatctccactgagggttggggcaacgctgctggaaagtcccagtacaccgtctccgctgcttaaattgtgggcgcggtccgttctttggctcaaggggaatgaaccaggggttcgagagatttgtgaaacattggcttgcgcaatcatcaattcctctccagaggagaggaaatatgatagcttcaagggctttgtaggggtgttgatttaggggatgctgccgctgctgttgcttgattcattggtgtttatctttctttcttcttgatttgttcagggccttgacacaccctggcttgtacatactttttttactcgtttcttgtagaaacgggtaaatcctgaatatacacgttgccttgtttctccctcgtttggatgacaatagcaattgcccggttatgaaccgaaaaaaaaactcctcatcttcttctgtgtttcttatcattgtgacaagtgacttttttttcttttcttacttaaatttatttttctaggttttcacaaaagtatatatattcttagccatgctggctgccccatcttacttagtaaacaggtgaaacttccaattatcgctgccaaacgcaggctcggatgtcggcggcggcgcgtggatggagatacataacattgtgttgcttaaaacaaggctaggttggctgggctggggctgaacagccggtggtaggatgatttatcgggcaaatatcaagacacacacacatcttcttaaaacgcccttttggtggcgaaggttcttaaccaagtgggtaccc

SEQ ID NO: 35 - DNA, 2527 bp, Cochliobolus carbonum
ctttttgtgaaggcacctccgtagacatcagtccttagatctgacttgcgcagcaccacacaagcagcagttattccttgggctccaatctggtcacaccaccaaacaaatccaacgggctaatatcgcaaggcagtgcagtaagcggtgttggcgtgagcattgtagctcgccttgaaagttggtccggcataacaatcttccacagtaaagaacgcacactgtttctccttattggtgtgtcaattaacgtccacatccattaagcctggatattcggctagccaagtggagtcttggaaagcttgtggaagacaataagcgcaggtaccttggctgcatatgcagtttccccagacagatgcactccacaattccttctgttccggagaaatcatcctcgcaaatcctaacggctttactacccctaccaagtggtcttatgccaagtcgatcgacgagtacggaccatcgctacggaatattctccacgaatcgtccatccccgcatgccaccacgtagccaaaccaacgaagaacagaaatggacccgtagaaccagcactggtaccggacatatcccgtattgcggtgaatcattatcagagctccgtggatccagtcattgttgtgctgcactaattactcgcgcgcagagtaaagcggtctacgtaccgagctatggttttgcgtgtgcttacttgcgtactgaacacagcccctgttgatatattaatatcggaaatttattcagcacaatgatgtcgcgtttcgagaattaggtggaacaagcataggccggctgttgttgtttcgccatgaactccctactcgcatttagccgctttttccccgtgcagaaaacgccaagtacctcaagcagcacaagaaccgaccaccgaagcttggaataaattaacccgaaacgcctggtccccacttttttactctatacggtaagactacgaatcattcaggccatgaccgacatgaacgacgcattgcaggtgcgattgcaggcgatgtttgattgcgttaacgccctattttcgggggttctccacaacgtggtgggtatgcggcttctagacgacgcctgccgttgtttgaggcgaattacattgtttagacgaaatgcgtgcgccgaatagtctagggcactctggggggacgatggattggtataagtagagctgggcccatgggcatatcttcgtatccatcacattcattcatttcagcttctccattctaccaaacaatcaaaccaatccttttagcaaccatgaagttttctctcatcaccatcctttccgccagcgctttggtcgctgcctctccctttgctgagccagaggctttccttgaggagcgccaggctgcgcagagcctcgatgccgccatgaaggccaagggcaggaagtacttcggtaccgccactgacccaggccgattcaaccagggcaagaacgctgccatcatcaaggccaactttggtcaaatcacccccgagaactccatgaagtgggatgctaccgagtctacccgtggcaaattcaccttcggtactgccgaccagaccgctaagttcgccaaggacaacggcaagctcatccgtggacacaccaccatctggcactcccagcttccttcatgggtttcttccatcaaggacaaggccaccttgaccactgtcatgcagaaccacatctcctccgtcatgggccacttcaagggccagatctacgcctgggacgtcatcaacgagatgttcgaggagaacggtaacttccgtgccagcgtcttctacaacgtcctcggtgaggactttgtccgcatcgccttcgaggctgccaagaaggctgaccccactgctaagcgttacatcaacgactacaagtaagcattgatctatgattaccaagtttttcgatatactgactgcaatatagccttgacactgccaactacgcaaagacccaggccatggccaagaacgtcaagaagtggatcgccgccggtatccccatcgacggtatcggttcgcagactcaccttaccgccggccagggtgccgctaccatcgacgccatgaagctcctctgcagcgttgcttccgagtgcgccatgaccgaggtcgacatccagaacgcccagcaggctgactggaccaacgtcaccaaggcctgccttaaccagaagaactgcgttggtatcaccgtctggggtgtcagggactccgactcctggaggccccagggcaaccctctcctcttcgacaacagctacaaccccaagcaggcttacaccactgtcctcaacgctctcaagtaaactgtttgcgatgctgggcatcctgggaaccaggaagactatgtcttttcttgctcccttacctatccttcttttcgacatattattgtaaatactttgattggtggtggagatattgtaaatatttgattgaaaggaatgaagaagttgatctttcccctataattgtgtgcttcttgatctctctactagt